(19) United States
(12) Patent Application Publication
(10) Pub. No.: US 2024/0228507 A1
(43) Pub. Date: Jul. 11, 2024

(54) GREEN SOLVENTS FOR CHEMICAL REACTIONS

(71) Applicant: ECOLE POLYTECHNIQUE FÉDÉRALE DE LAUSANNE (EPFL), Lausanne (CH)

(72) Inventors: Anastasiia KOMAROVA, Chavannes-près-Renens (CH); Jeremy LUTERBACHER, Chavannes-près-Renens (CH)

(73) Assignee: ECOLE POLYTECHNIQUE FÉDÉRALE DE LAUSANNE (EPFL), Lausanne (CH)

(21) Appl. No.: 18/287,063

(22) PCT Filed: Apr. 14, 2022
(86) PCT No.: PCT/EP2022/060153

§ 371 Date: Oct. 16, 2023

(30) Foreign Application Priority Data
Apr. 18, 2021 (EP) 21169040.9

(51) Int. Cl. C07D 493/14; C07D 493/04; C07D 493/22

(52) U.S. Cl. CPC C07D 493/14; C07D 493/04; C07D 493/22

(57) ABSTRACT

Use of a compound of the general formula (I), (II), (III), (IV), (V) or (VI)

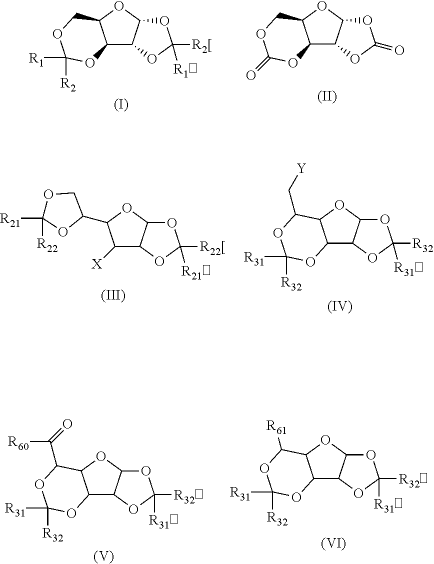

as solvent for chemical reactions, for recrystallizations or extractions.

The present invention relates to the use of xylose derivatives as solvent, preferably as polar for chemical reactions or for recrystallizations or for extractions.

In recent decades, the gradual depletion of fossil fuel resources, increase in global energy consumption, and environmental issues have driven the development of new materials and technologies that utilize renewable biological sources such as biomass or food waste. One of the cutting-edge topics in this area is the development of bio-based solvents that are considered as greener alternatives to petroleum-derived solvents. In principle, bio-based solvents do not result in a net increase of carbon dioxide in the atmosphere at the end of their lifetimes and thus ensure less environmental damages. In a further aspect, bio-based solvents are typically less toxic. This creates efficiency savings due to the reduced costs of hazardous waste disposal and safety management.

The development of viable replacements for conventional polar aprotic solvents (PAS) has recently been identified as a key green chemistry research area according to ACS Green Chemistry Institute® Pharmaceutical Roundtable. This class of organic solvents is considered as one of the most difficult to replace solvents due to their unique solubilizing properties and the ability to provide strong interactions with the active pharmaceutical ingredients. Research in this field is also highly driven by regulatory initiatives. For instance, European Union REACH (Registration, Evaluation, Authorisation and Restriction of Chemicals) recently has restricted the industrial use of N-methylpyrrolidinone (NMP), and listed others of the most common PAS like N,N-dimethylacetamide (DMAc) or N,N-dimethylformamide (DMF) as substances of very high concern due to their severe reproductive toxicity.

Up to now, a number of bio-based solvents that offer a potential to replace highly toxic PAS have been developed and some of them commercialized. Typical examples of bio-based alternatives to PAS include 2-methyltetrahydrofuran (2-MeTHF) and γ-valerolactone (GVL).

2-MeTHF is a solvent that can be produced through two-step hydrogenation of furfural, which is a renewable furan that can be obtained from biomass-derived carbohydrates (C. J. Clarke, W. C. Tu, O. Levers, A. Brohl and J. P. Hallett, Chem. Rev., 2018, 118, 747-800). Usage of this solvent grew rapidly in past decades and now 2-MeTHF is mostly used as a renewable alternative to THF due to its lower toxicity and greatly reduced solvent emissions and waste streams. 2-MeTHF has also proved to be a suitable alternative to some PAS, such as DMSO, acetonitrile, acetone in a series of reactions. However, due to its high flammability, the use of 2-MeTHF is problematic.

γ-valerolactone is biorenewable and (GVL) another biodegradable solvent that can be produced from cellulose or hemicellulose via levulinic acid pathway (E. Ismalaj, G. Strappaveccia, E. Ballerini, F. Elisei, O. Piermatti, D. Gelman and L. Vaccaro, ACS Sustain. Chem. Eng., 2014, 2, 2461-2464). Although being a good alternative, it has a limited stability which of course limits its use. Furthermore, GVL has limited applications at an industrial scale due to high production costs, which is associated with its many production steps form biomass.

Schmidt and Nieswandt, Chemische Berichte, 82, No. 1, 1949, pages 1 to 94 disclose the determination of the molecular weight by cryoscopy of a specific lacton by using dimethylen-xylose as a solvent because of its structural similarity. A chemical reaction was not involved, i.e. dimethylen-xylose has not been used as solvent for chemical reactions.

Y. M. Questell-Santiago, R. Zambrano-Varela, M. Talebi Amiri and J. S. Luterbacher, Nat. Chem. 2018, 10, 1222-1228 discloses that Diformylxylose (DFX) can be directly synthesized from D-Xylose in the presence of 37 wt % aqueous solution of formaldehyde and 37 wt % aqueous solution of HCL using 1,4 dioxane as solvent. N-hexane is used as extraction solvent.

The problem of the present invention is to provide bio-based solvents with different properties for the replacement of hazardous and/or petroleum-based conventional analogues.

The problem is solved by the solvent compounds according to claim 1 as well as by the method according to claim 17 as well as by a compound according to claim 23. Further preferred embodiments are subject of the dependent claims.

It was found that a compound selected from the group consisting of the general formula (I), (II), (III), (IV), (V) and/or (VI)

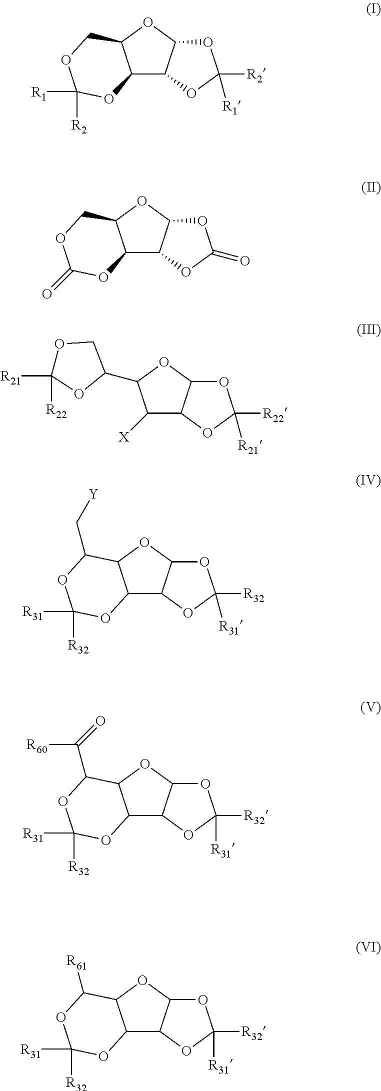

wherein R₁ and R₁′, R₂₁ and R₂₁′, R₃₁ and R₃₁′ are the same and are hydrogen, a linear or branched C₁ to C₁₈ alkyl, a linear or branched C₂ to C₁₈ alkenyl, a linear or branched C₁ to C₁₀ alkoxy, a linear or branched C₁ to C₉ alkanoyloxy, a linear or branched C₁ to C₉ alkoxycarbonyl, aminocarbonyl, hydroxycarbonyl, a linear or branched C₁ to C₉ alkoxyalkyl, an unsubstituted cycloalkyl, a linear or branched C₁ to C₆ alkyl substituted cycloalkyl, preferably an unsubstituted cyclohexyl, an unsubstituted C₆ to C₁₂ aryl, a linear or branched C₁ to C₄ alkyl substituted C₆ to C₁₂ aryl, or C₇ to C₁₂ aralkyl,

R₂ and R₂′, R₂₂ and R₂₂′, R₃₂ and R₃₂′ are the same and are hydrogen, a linear or branched C₁ to C₁₈ alkyl, a linear or branched C₁ to C₉ alkoxyalkyl, or R₂ and R₂′ form together with R₁ and R₁′ respectively, R₂₂ and R₂₂′ form together with R₂₁ and R₂₁′ respectively, R₃₂ and R₃₂′ form together with R₁ and R₁′ respectively, a 5 or 6 membered unsaturated or saturated carbocyclic ring optionally comprising 1 or 2 oxygen atoms,

X is selected from the group consisting of hydrogen, fluoro, chloro, bromo, iodo, a linear or branched C₁ to C₉ alkanoyloxy, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl,

Y is selected from the group consisting of hydrogen, fluoro, chloro, bromo, iodo, a linear or branched C₂ to C₁₀ alkenyl, a sulfonate, a linear or branched C₁ to C₁₈ alkyl, a C₁ to C₁₈ alkenyl, and OR₅₀, wherein R₅₀ is selected from the group consisting of hydrogen, a linear or branched C₁ to C₁₈ alkyl, and a linear or branched C₁ to C₉ alkylcarbonyl,

R₆₀ is selected from the group consisting of hydrogen, hydroxy and C₁ to C₁₈ alkoxy and

R₆₁ is selected from the group consisting of hydrogen, hydroxy, C₁ to C₁₀ alkylsulfonyl-C₁ to C₅ alkenyl (preferably methylsulfonylmethylenyl) and C₁ to C₁₈ alkyl.

can be used as solvent, preferably as polar for chemical reactions or for recrystallizations.

The term “linear C₁ to C₉ alkoxyalkyl” denotes a group of the formula —(CH₂)_{q}—O—CᵣH₂ᵣ₊₁, wherein q, r and r′ are independently from each other 1 to 9. Preferred embodiments of linear C₁ to C₉ alkoxyalkyl are —CH₂OCH₃, —CH₂CH₂OCH₃, —CH₂OCH₂CH₃, —CH₂CH₂OCH₂CH₃. In case of a branched C₁ to C₉ alkoxyalkyl it denotes a corresponding branched residue such as for example —(CH₂)_{q-1}—(CH)—(O—CᵣH₂ᵣ₊₁)₂ or —(CH₂)_{q-1}—(CH)—(O—CᵣH₂ᵣ₊₁) (C_{r′}H_{2r′+1}). Preferred embodiment of such a branched C₁ to C₉ alkoxyalkyl are —CH(OCH₃)₂, —CH(OCH₂CH₃)₂ and —CH(OCH₃)(CH₃).

The term “oxo” denotes a C═O bond.

The term “alkanoyloxy” denotes an ester group —OC(O)R, R being an organyl residue, preferably an alkyl or aryl.

The term “Hydroxycarbonyl” means a C(O)OH radical or a carboxylic acid —CO—OH. The term “Aminocarbonyl” or “Carbamoyl” denotes a —C(O)NR₂ radical, wherein R₁ and R₁′, R₂₁ and R₂₁′, R₃₁ and R₃₁′ are independently from each other hydrogen or C₁ to C₁₈ alkyl, preferably both hydrogen.

The term “Alkoxycarbonyl” means a —C(0)OR radical wherein means a —C(0)OR radical where R is alkyl or aryl.

The term “sulfonate” denotes an ester of sulfonic acid such as mesylate and tosylate.

The term “carboxy” denotes a carboxylate or a carboxylic acid depending on the pH.

The term “C₁ to C₉ alkoxyalkyl” denotes a group of the formula —(CH₂)_{q}—O—CᵣH₂ᵣ₊₁, wherein q and r are independently from each other 1 to 9.

According to a preferred embodiment of the present invention the compound is selected from the group of the general formula (I)

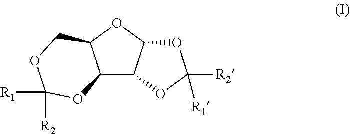

wherein R₁ and R₁′ are the same and are a linear or branched C₁ to C₁₀ alkyl,

R₂ and R₂′ are the same and are a linear or branched C₁ to C₁₀ alkyl or

R₂ and R₂′ form together with R₁ and R₁′ respectively, a 5 or 6 membered unsaturated or saturated carbocyclic ring optionally comprising 1 or 2 oxygen atoms,

preferably wherein R₂ and R₂′ form together with R₁ and R₁′ respectively, a 5 or 6 membered unsaturated or saturated carbocyclic ring optionally comprising 1 or 2 oxygen atoms.

One aspect of the present invention relates to the use of compounds of the general formula (I), (II), (III), or (IV), (V) or (VI) as polar aprotic solvents, thus solvents which do not contain at least one hydrogen atom connected directly to the electronegative oxygen atom. In other words, this aspect of the present invention relates to the compounds of formula I and II, as well as to compounds of the formula III, wherein X is selected from the group consisting of hydrogen, fluoro, chloro, bromo, iodo, a linear or branched C₁ to C₉ alkanoyloxy, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl and to compounds of the formula IV, V, VI Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl and a linear or branched C₁ to C₉ alkylcarbonyl, and

R₆₀ is selected from the group consisting of hydrogen, hydroxy and C₁ to C₁₈ alkoxy and

R₆₁ is selected from the group consisting of hydrogen, hydroxy, C₁ to C₁₀ alkylsulfonyl-C₁ to C₅ (preferably methylsulfonylmethyl), alkenyl and C₁ to C₁₈ alkyl.

A second aspect of the present invention relates to the use of compounds of the general formula (III) or (IV) as polar protic solvents, thus solvents which contain at least one hydrogen atom connected directly to the electronegative oxygen atom. In other words, this aspect of the present invention relates to the compounds of formula III, wherein X is OR₅₀, wherein R₅₀ is hydrogen and to compounds of the formula IV, wherein Y is OR₅₀, wherein R₅₀ is hydrogen.

In a further aspect of the present invention X is hydroxy leading to a particularly good solubility of the compounds with ability to form hydrogen bonding.

The polar aprotic solvents according to the present invention demonstrate similar performance to conventional polar aprotic solvents (like DMF, NMP and DMSO) in chemical reactions. This is remarkable as the compounds of the general formula (I) or (II) do not contain any nitrogen or sulfur heteroatoms, which are typically found in polar aprotic solvents. Those heteroatoms are known to lead to environmental pollution, and their absence is potentially beneficial. Further, it was found that said compounds have a non-mutagenic and non-volatile nature indicating that this solvent could be safe for human health, which would make it truly “green”. Furthermore, as they can be produced directly from lignocellulosic biomass, their production is likely to be both sustainable and economically feasible.

The polar aprotic solvents according to the present invention, and in particular the compounds of formula (I) or (II) have a unique combination of solvent properties such as polarity, boiling point, melting point, viscosity and poor miscibility with water and can therefore be easily implemented in industry and laboratory routine. Polarity controls both the solubility of different components and the productivity of a reaction through kinetic and thermodynamic effects, whereas physical properties are responsible for separation, handling, and disposal of solvent. Due to their solvent properties, polar aprotic solvents according to the present invention, and in particular the compounds of formula (I) or (II) can satisfy different needs over a variety of chemical sectors. Due to their overall efficacy, toxicity, and sustainability, they can positively influence the cost of the final product.

The polar protic solvents according to the present invention demonstrate similar performance to conventional polar protic solvents (like DMF, NMP, DMAc, dimethyl sulfoxide (DMSO)) in chemical reactions. Since they are strong hydrogen-bond donors, they are very effective at stabilizing any ions. In addition, they can also be used as industrial solvents to help create inks, resins, adhesives, and dyes.

The compounds as defined in claim 1 can be used as solvents, preferably as polar solvents for chemical reactions, for recrystallizations or for extractions since they have high boiling points and do not react with the starting compounds in chemical reactions.

According to a preferred embodiment of the invention, the compounds (I) to (VI) as defined in claim 1, in particular Diformylxylose, can be used as solvent, in particular as polar solvent, for chemical reactions like
Cross-coupling reactions (e.g. Heck reaction, Sonogashira reaction, Negishi coupling, Wurtz, Grignard, Castro-Stephens, Corey-House, Kumada coupling, Stille coupling, Suzuki coupling, Hiyama coupling, Fukuyama coupling, dehydrohalide coupling, etc.)
Alkylation reactions (e.g. Menshutkin reaction)
Hydrogenation reactions
Enzymatic reactions, such as polycondensations for the synthesis of polyesters e.g. in the presence of lipases, or
the following reactions: amidations, esterifications, Aza-Michael addition, Krapcho dealkoxycarbonylation, Beckmann rearrangement and Diels Alder cycloaddition.

Moreover, the compounds (I) to (VI) as defined in claim 1, in particular Diformylxylose, can be used
as at least one component of battery electrodes, e.g. as replacement for dioxalane,
for synthesis of metal-organic frameworks (MOFs)
for preparation of electrodes, e.g. as substitute for NMP,
liquid-phase exfoliations, in particular as substitute for NMP, e.g. as substitute for NMP in liquid exfoliation of MoS₂ particles,
solvent for storage due to high viscosity and melting point,
solvent for biomass pretreatment and biomass processing, in particular for aldehyde-assisted biomass fractionation,
for solid-Phase Peptide Synthesis, e.g. as replacement for DMF, or as
for Microfiltration membrane preparation, e.g. as replacement for Tetrahydrofuran (THF), DMAC, NMP
component of Deep eutectic solvents, in particular when mixed with Phenol, Propyl guaiacol, Catechol, Resorcin, Glyoxylic acid, Lactic acid).

In one embodiment of the present invention, the compound of formula (I) is a compound of formula (Ia)

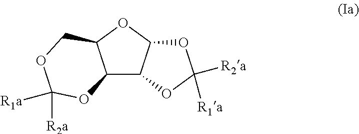

wherein R₁ₐ and R₁ₐ′ are the same and are hydrogen, a linear or branched C₁ to C₁₈ alkyl and R₂ₐ and R₂ₐ′ are the same and are hydrogen, or a linear or branched C₁ to C₁₀ alkyl. Preferably, each of R₂ₐ and R₂ₐ′ is hydrogen.

The term linear C₁ to C₁₈ alkyl stands for an alkyl chain which is preferably selected from the group consisting of methyl, ethyl, propyl, butyl, pentyl, hexyl, heptyl, octyl, nonyl and decyl. In a branched alkyl chain, branching occurs preferably either directly in α-position adjacent to the ring system or in Q-position, that is at the end of the alkyl chain. Compounds of formula (Ia) are moderately stable and can withstand harsh reaction conditions.

In another embodiment of the present invention, the compound of formula (I) is a compound of formula (Ib)

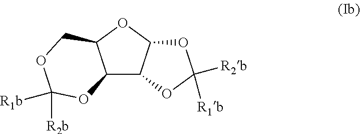

wherein wherein R_{1b} and R_{1b}′ are both linear or branched C₂ to C₁₀ alkenyl and R_{2b} and R_{2b}′ are both hydrogens. These structures are preferred for reactions requiring ether solvent of medium polarity (e.g. Wurtz reaction, Grignard reaction, etc.).

In another embodiment of the present invention, the compound of formula (I) is a compound of formula (Ic)

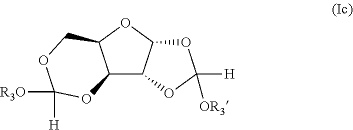

wherein R₃ and R₃′ are both a linear or branched C₁ to C₁₀ alkyl. The term linear C₁ to C₁₀ alkyl stands for an alkyl chain as defined above. Thus, in other words, compounds of formula (Ic) stand for compounds of formula (I), wherein R₁ and R₁′ are a linear or branched C₁ to C₁₀ alkoxy and each of R₂ and R₂′ are hydrogen. Higher oxygen content of such solvents increases their hydrogen-bond accepting ability or basicity, which makes them advantageous solvents for Michael addition, specifically.

Also, high basicity of solvent is typically required for solubilization of complex targets such as lignin and cellulose.

Moreover, highly oxygenated solvents can be used to tune CH— aryl interactions in solution and favour formation of one of possible conformers.

In another embodiment of the present invention the compound of formula (I) is a compound of formula (Id)

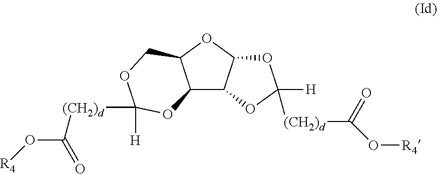

wherein R₄ and R₄′ are both a linear or branched C₁ to C₉ alkyl. The term linear C₁ to C₁₀ alkyl stands for an alkyl chain as defined above. Thus, in other words, compounds of formula (Id) stand for compounds of formula (I), wherein R₁ and R₁′ are a linear or branched C₁ to C₉ alkoxycarbonyl and each of R₂ and R₂′ are hydrogen, preferably a linear C₁ to C₉ alkoxycarbonyl. Preferably R₁ and R₁′ are selected from the group consisting of methoxycarbonyl and ethoxycarbonyl. Due to the presence of the ester group, compounds of formula (Id) are especially good solvents for positively charged starting compounds and form strong hydrogen bonds.

Most preferably in the compound of formula (Id), each of R₄ and R₄′ is methyl, and d is 0 which corresponds to compound 14.

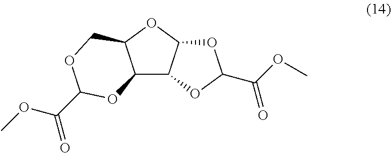

Compound (14) shows a high rate constant for Menshutkin reaction and Heck reaction.

In another embodiment of the present invention, the compound of formula (I) is a compound of formula (Ie)

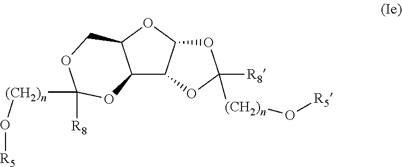

wherein n is a number between 1 and 9 and R₅ and R₅′ are both a linear or branched C₁ to C₉ alkyl. Thus, in other words, compounds of formula (Ie) stand for compounds of formula (I), wherein R₁ and R₁′ are a linear or branched C₁ to C₉ alkoxyalkyl, and each of R₂ and R₂′ are hydrogen.

Preferably, in the compound of formula (Ie), n is 1 or 2, and each of R₅ and R₅′ is selected from the group consisting of methyl, ethyl, propyl, isopropyl or tert-butyl. Compounds of formula (Ie) relatively stable and can withstand harsh reaction conditions.

In another embodiment of the present invention, the compound of formula (I) is a compound of formula (If)

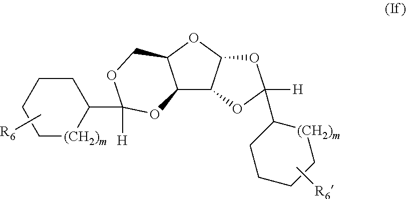

wherein m is a number between 0 and 6 and R₆ and R₆′ are both a linear or branched C₁ to C₆ alkyl. R₆ and R₆′ may be present one or more times at any position of the cycloalkyl residue. Thus, in other words, compounds of formula (If) stand for compounds of formula (I), wherein R₁ and R₁′ are unsubstituted cycloalkyl or a linear or branched C₁ to C₆ alkyl substituted cycloalkyl, and each of R₂ and R₂′ are hydrogen. Most preferably, in the compound of formula (If), m is 1 and the cycloalkyl is unsubstituted, that is, cyclohexyl. It's relatively low polarity can be advantageous for solvating non-polar resins/films, and other non- or middle-polar substrates. Also, similar to cyclohexane, it can be used for recrystallizations (as many organic compounds exhibit good solubility in hot cyclohexane and poor solubility at low temperatures).

In another embodiment of the present invention, the compound of formula (I) is a compound of formula (Ig)

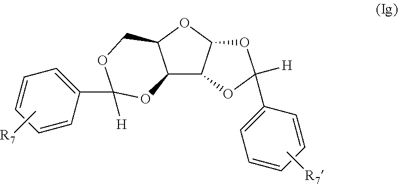

wherein R₇ and R₇′ are both a linear or branched C₁ to C₄ alkyl and R₇ and R₇′ may be optionally present one or more times at any position of the phenyl. Thus, in other words, compounds of formula (Ig) stand for compounds of formula (I) wherein R₁ and R₁′ are an unsubstituted C₆ aryl (i.e., phenyl) or a C₁ to C₄ alkyl substituted C₆ aryl (i.e., phenyl) and each of R₂ and R₂′ are hydrogen. Most preferably, in the compound of formula (If), the C₆ aryl is unsubstituted, that is, phenyl. Due to the presence of aromatic ring, these solvents can be effectively used for dissolution of pigments/dyes, rubber, disinfectants, silicone sealants, substrates with pi-bonds, etc. Like toluene, such solvents could be used for carbon nanomaterials, including nanotubes and fullerenes.

In another embodiment of the present invention, the compound of formula (I) is a compound of formula (Ih)

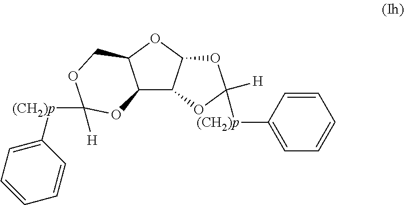

wherein p is a number between 1 and 6. Thus, in other words, compounds of formula (Ih) stand for compounds of formula (I), wherein R₁ and R₁′ are C₇ to C₁₂ aralkyl, wherein the aryl group of the aralkyl is phenyl, and each of R₂ and R₂′ are hydrogen.

In another embodiment of the present invention, the compound of formula (III) is a compound of formula (X),

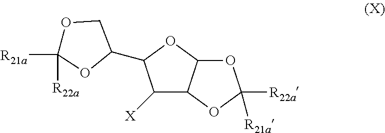

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₀ alkenyl, a linear or branched C₂ to C₁₀ alkenyl, a sulfonate and OR₅₀, wherein R₅₀ is selected from the group of hydrogen, a linear or branched C₁ to C₁₈ alkyl, and a linear or branched C₁ to C₉ alkylcarbonyl, and R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia, and in particular a compound of formula Xa to Xg:

| | | |
|---|---|---|
| Compound of formula Xa | 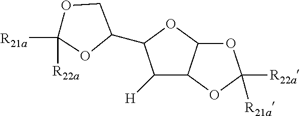 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia |
| Compound of formula Xb | 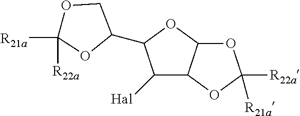 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia and wherein Hal is selected from the group consisting of fluoro, chloro, bromo and iodo |
| Compound of formula Xc | 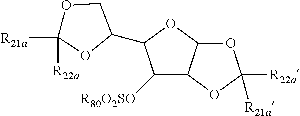 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia and wherein R₈₀ is methyl or p- methylphenyl |
| Compound of formula Xd | 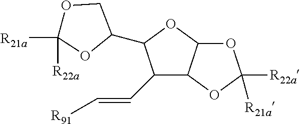 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia and wherein R₉₁ is a linear or branched C₁-C₁₈ alkyl, thus resulting in an alkenyl |
| Compound of formula Xe | 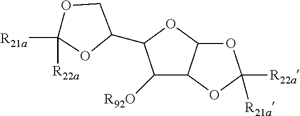 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia and wherein R₉₂ is a linear or branched C₁-C₁₈ alkyl, thus resulting in a linear or branched alkoxy group |
| Compound of formula Xf | 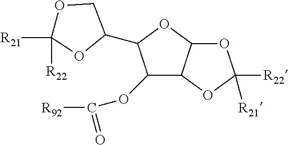 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia and wherein R₉₂ is a linear or branched C₁-C₁₈ alkyl, resulting in a linear or branched alkanoyloxy group |
| Compound of formula Xg | 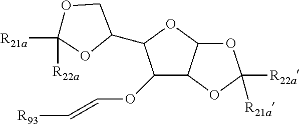 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia and wherein R₉₃ is a linear or branched C₁-C₁₈ alkyl, thus resulting in a linear or branched alkenyloxy |

In another embodiment of the present invention, the compound of formula (III) is a compound of formula (XI),

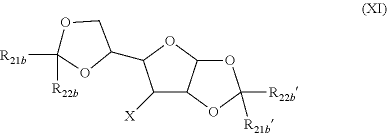

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and R_{21b}, R_{21b}′, R_{22b} and R_{22b}′ have the same definitions as R_{1b}, R_{1b}′, R_{2b} and R_{2b}′ in compound of formula Ib, and in particular a compound of formula XIa to XIg:

| | | |
|---|---|---|
| Compound of formula XIa | 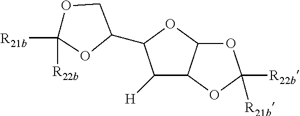 | Wherein R_{21b}, R_{21b}′, R_{22b} and R_{22b}′ have the same definitions as R_{1b}, R_{1b}′, R_{2b} and R_{2b}′ in compound of formula Ib |
| Compound of formula XIb | 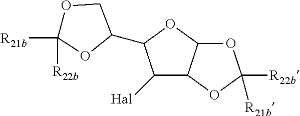 | Wherein R_{21b}, R_{21b}′, R_{22b} and R_{22b}′ have the same definitions as R_{1b}, R_{1b}′, R_{2b} and R_{2b}′ in compound of formula Ib and wherein Hal is selected from the group consisting of fluoro, chloro, bromo and iodo |
| Compound of formula XIc | 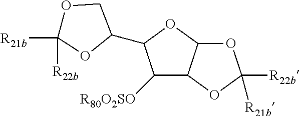 | Wherein R_{21b}, R_{21b}′, R_{22b} and R_{22b}′ have the same definitions as R_{1b}, R_{1b}′, R_{2b} and R_{2b}′ in compound of formula Ib and wherein R80 is methyl or p- methylphenyl |
| Compound of formula XId | 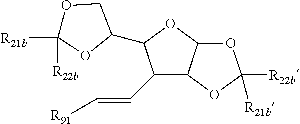 | Wherein R_{21b}, R_{21b}′, R_{22b} and R_{22b}′ have the same definitions as R_{1b}, R_{1b}′, R_{2b} and R_{2b}′ in compound of formula Ib and wherein R₉₁ is a linear or branched C₁-C₁₈ alkyl, thus resulting in an alkenyl |
| Compound of formula XIe | 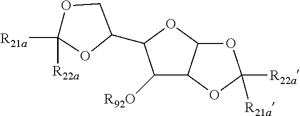 | Wherein R_{21b}, R_{21b}′, R_{22b} and R_{22b}′ have the same definitions as R_{1b}, R_{1b}′, R_{2b} and R_{2b}′ in compound of formula Ib and wherein R₉₂ is a linear or branched C₁-C₁₈ alkyl, thus resulting in a linear or branched alkoxy |
| Compound of formula XIf | 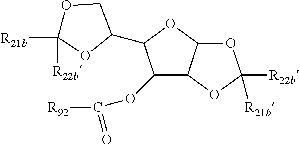 | Wherein R_{21b}, R_{21b}′, R_{22b} and R_{22b}′ have the same definitions as R_{1b}, R_{1b}′, R_{2b} and R_{2b}′ in compound of formula Ib and wherein R₉₂ is a linear or branched C₁-C₁₈ alkyl, resulting in a linear or branched alkanoyloxy group |
| Compound of formula XIg | 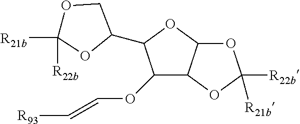 | Wherein R_{21b}, R_{21b}′, R_{22b} and R_{22b}′ have the same definitions as R_{1b}, R_{1b}′, R_{2b} and R_{2b}′ in compound of formula Ib and wherein R₉₃ is a linear or branched C₁-C₁₈ alkyl, thus resulting in a linear or branched alkenyloxy |

In another embodiment of the present invention, the compound of formula (III) is a compound of formula (XII),

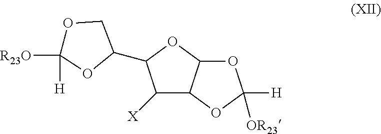

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl and a linear or branched C₁ to C₉ alkylcarbonyl, and R₂₃ and R₂₃′ have the same definitions as R₃, R₃′ in compound of formula Ic, and in particular a compound of formula XIIa to XIIg:

| | | |
|---|---|---|
| Compound of formula XIIa | 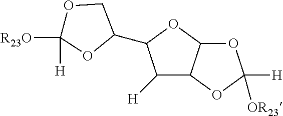 | Wherein R₂₃ and R₂₃′ have the same definitions as R₃, R₃′ in compound of formula Ic |
| Compound of formula XIIb | 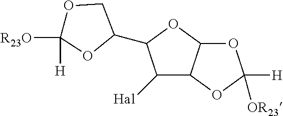 | Wherein R₂₃ and R₂₃′ have the same definitions as R₃, R₃′ in compound of formula Ic and wherein Hal is selected from the group consisting of fluoro, chloro, bromo and iodo |
| Compound of formula XIIc | 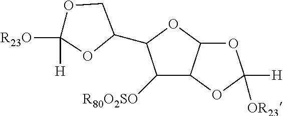 | Wherein R₂₃ and R₂₃′ have the same definitions as R₃, R₃′ in compound of formula Ic and wherein R₈₀ is methyl or p- methylphenyl |
| Compound of formula XIId | 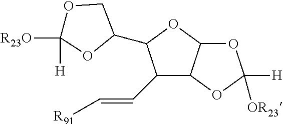 | Wherein R₂₃ and R₂₃′ have the same definitions as R₃, R₃′ in compound of formula Ic and wherein R₉₁ is a linear or branched C₁-C₁₈ alkyl, thus resulting in an alkenyl |
| Compound of formula XIIe | 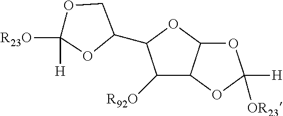 | Wherein R₂₃ and R₂₃′ have the same definitions as R₃, R₃′ in compound of formula Ic and wherein R₉₂ is a linear or branched C₁-C₁₈ alkyl, thus resulting in a linear or branched alkoxy |
| Compound of formula XIIf | 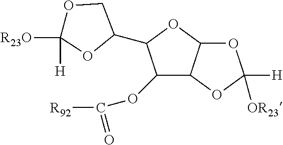 | Wherein R₂₃ and R₂₃′ have the same definitions as R₃, R₃′ in compound of formula Ic and wherein R₉₂ is a linear or branched C₁-C₁₈ alkyl, thus resulting in a linear or branched alkanoyloxy group |
| Compound of formula XIIg | 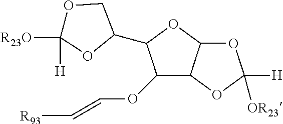 | Wherein R₂₃ and R₂₃′ have the same definitions as R₃, R₃′ in compound of formula Ic and wherein R₉₃ is a linear or branched C₁-C₁₈ alkyl, thus resulting in a linear or branched alkenyloxy |

In another embodiment of the present invention, the compound of formula (III) is a compound of formula (XIII),

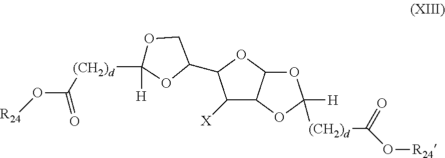

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and R₂₄ and R₂₄′ have the same definitions as R₄ and R₄′ in compound of formula Id, and d is 0, 1 or 2, and in particular a compound of formula XIIIa to XIIIg:

| | | |
|---|---|---|
| Compound of formula XIIIa | 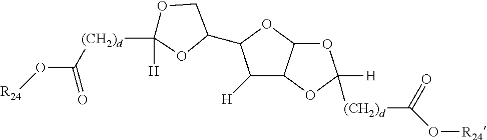 | Wherein R₂₄ and R₂₄′ have the same definitions as R₄, R₄′ in compound of formula Id and d is 0, 1 or 2 |
| Compound of formula XIIIb | 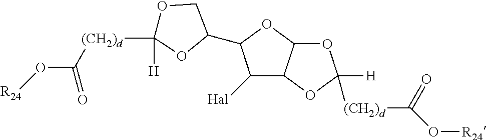 | Wherein R₂₄ and R₂₄′ have the same definitions as R₄, R₄′ in compound of formula Id, d is 0, 1 or 2 and wherein Hal is selected from the group consisting of fluoro, chloro, bromo and iodo |
| Compound of formula XIIIc | 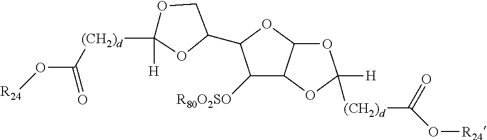 | Wherein R₂₄ and R₂₄′ have the same definitions as R₄, R₄′ in compound of formula Id, d is 0, 1 or 2 and wherein R₈₀ is methyl or p-methylphenyl |
| Compound of formula XIIId | 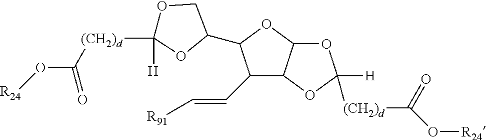 | Wherein R₂₄ and R₂₄′ have the same definitions as R₄, R₄′ in compound of formula Id, d is 0, 1 or 2 and wherein R₉₁ is a linear or branched C₁-C₁₈ alkyl, thus resulting in an alkenyl |
| Compound of formula XIIIe | 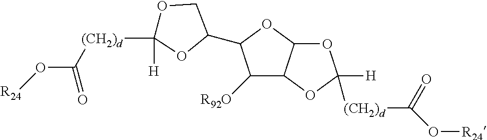 | Wherein R₂₄ and R₂₄′ have the same definitions as R₄, R₄′ in compound of formula Id, d is 0, 1 or 2 and wherein R₉₂ is a linear or branched C₁-C₁₈ alkyl, thus resulting in a linear or branched alkoxy |
| Compound of formula XIIIf | 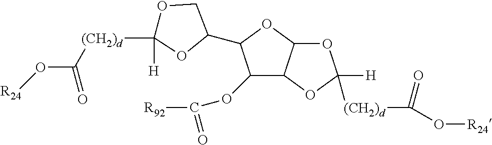 | Wherein R₂₄ and R₂₄′ have the same definitions as R₄, R₄′ in compound of formula Id, d is 0, 1 or 2 and wherein R₉₂ is a linear or branched C₁-C₁₈ alkyl, resulting in a linear or branched alkanoyloxy group |
| Compound of formula XIIIg | 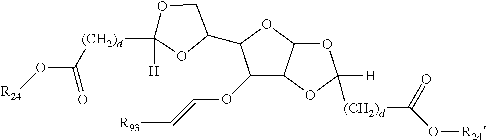 | Wherein R₂₄ and R₂₄′ have the same definitions as R₄, R₄′ in compound of formula Id, d is 0, 1 or 2 and wherein R₉₃ is a linear or branched C₁-C₁₈ alkyl, resulting in a linear or branched alkenyloxy |

In another embodiment of the present invention, the compound of formula (III) is a compound of formula (XIV),

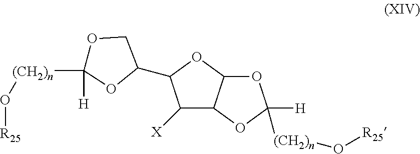

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₈ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and R₂₅ and R₂₅′ have the same definitions as R₅ and R₅′ in compound of formula Ie, and n is 1 or 2, and in particular a compound of formula XIVa to XIVg:

| | | |
|---|---|---|
| Compound of formula XIVa | 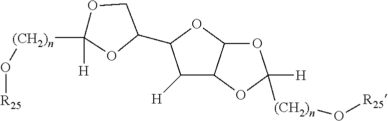 | Wherein R₂₅ and R₂₅′ have the same definitions as R₅, R₅′ in compound of formula Ie and n is 1 or 2 |
| Compound of formula XIVb | 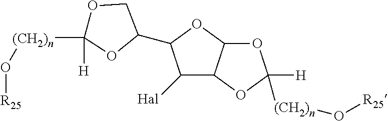 | Wherein R₂₅ and R₂₅′ have the same definitions as R₅, R₅′ in compound of formula Ie, n is 1 or 2 and wherein Hal is selected from the group consisting of fluoro, chloro, bromo and iodo |
| Compound of formula XIVc | 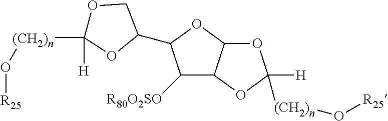 | Wherein R₂₅ and R₂₅′ have the same definitions as R₅, R₅′ in compound of formula Ie, n is 1 or 2 and wherein R₈₀ is methyl or p-methylphenyl |
| Compound of formula XIVd | 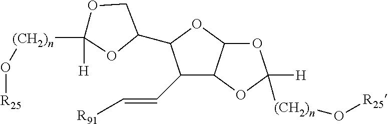 | Wherein R₂₅ and R₂₅′ have the same definitions as R₅, R₅′ in compound of formula Ie, n is 1 or 2 and wherein R₉₁ is a linear or branched C₁-C₁₈ alkyl, thus resulting in an alkenyl |
| Compound of formula XIVe | 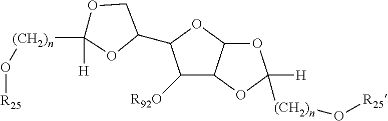 | Wherein R₂₅ and R₂₅′ have the same definitions as R₅, R₅′ in compound of formula Ie, n is 1 or 2 and wherein R₉₂ is a linear or branched C₁-C₁₈ alkyl, thus resulting in a linear or branched alkoxy |
| Compound of formula XIVf | 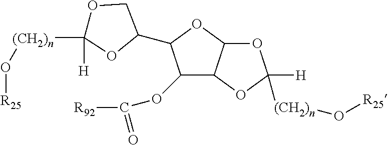 | Wherein R₂₅ and R₂₅′ have the same definitions as R₅, R₅′ in compound of formula Ie, n is 1 or 2 and wherein R₉₂ is a linear or branched C₁-C₁₈ alkyl, thus resulting in a linear or branched alkanoyloxy group |
| Compound of formula XIVg | 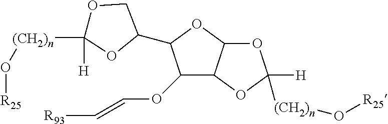 | Wherein R₂₅ and R₂₅′ have the same definitions as R₅, R₅′ in compound of formula Ie, n is 1 or 2 and wherein R₉₃ is a linear or branched C₁-C₁₈ alkyl, thus resulting in a linear or branched alkenyloxy |

In another embodiment of the present invention, the compound of formula (III) is a compound of formula (XV),

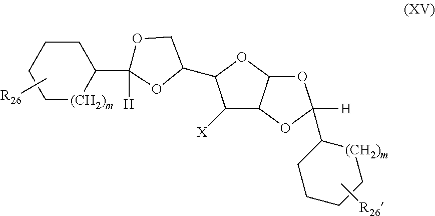

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and R₂₆ and R₂₆′ have the same definitions as R₁ₐ and R₁ₐ′ in compound of formula If, and m is a number between 1 and 6, and in particular a compound of formula XVa to XVg:

| | | |
|---|---|---|
| Compound of formula XVa | 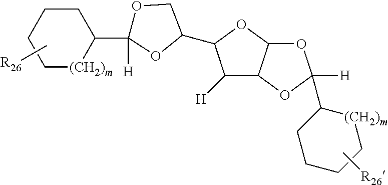 | Wherein R₂₆ and R₂₆′ have the same definitions as R₆, R₆′ in compound of formula If and m is a number between 1 and 6 |
| Compound of formula XVb | 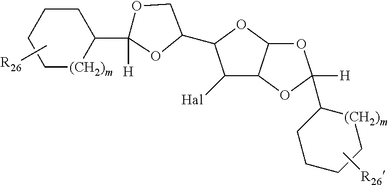 | Wherein R₂₆ and R₂₆′ have the same definitions as R₆, R₆′ in compound of formula If, m is a number between 1 and 6 and wherein Hal is selected from the group consisting of fluoro, chloro, bromo and iodo |
| Compound of formula XVc | 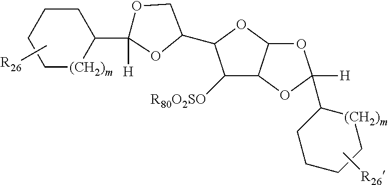 | Wherein R₂₆ and R₂₆′ have the same definitions as R₆, R₆′ in compound of formula If, m is a number between 1 and 6 and wherein R₈₀ is methyl or p-methylphenyl |
| Compound of formula XVd | 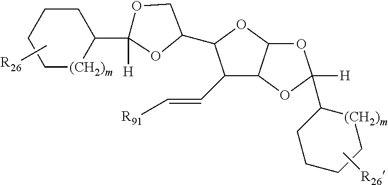 | Wherein R₂₆ and R₂₆′ have the same definitions as R₆, R₆′ in compound of formula If, m is a number between 1 and 6 and wherein R₉₁ is a linear or branched C₁-C₁₈ alkyl, thus resulting in an alkenyl |
| Compound of formula XVe | 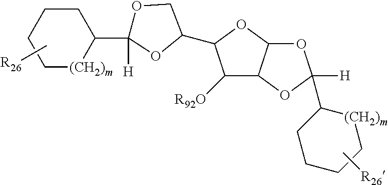 | Wherein R₂₆ and R₂₆′ have the same definitions as R₆, R₆′ in compound of formula If, m is a number between 1 and 6 and wherein R₉₂ is a linear or branched C₁-C₁₈ alkyl, thus resulting in a linear or branched alkoxy |
| Compound of formula XVf | 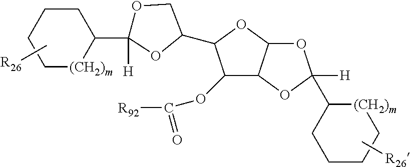 | Wherein R₂₆ and R₂₆′ have the same definitions as R₆, R₆′ in compound of formula If, m is a number between 1 and 6 and wherein R₉₂ is a linear or branched C₁-C₁₈ alkyl, resulting in a linear or branched alkanoyloxy group |
| Compound of formula XVg | 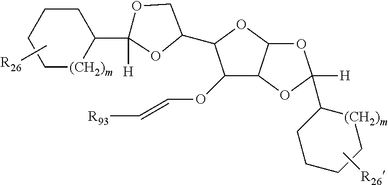 | Wherein R₂₆ and R₂₆′ have the same definitions as R₆, R₆′ in compound of formula If, m is a number between 1 and 6 and wherein R₉₃ is a linear or branched C₁-C₁₈ alkyl, resulting in a linear or branched alkenyloxy |

In another embodiment of the present invention, the compound of formula (III) is a compound of formula (XVI),

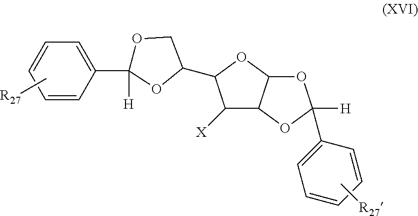

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and R₂₇ and R₂₇′ have the same definitions as R₇ and R₇′ in compound of formula Ig, and in particular a compound of formula XVIa to XVIg:

| | | |
|---|---|---|
| Compound of formula XVIf | 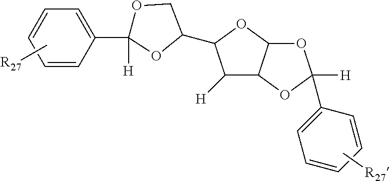 | Wherein R₂₇ and R₂₇′ have the same definitions as R₇, R₇′ in compound of formula Ig |
| Compound of formula XVIb | 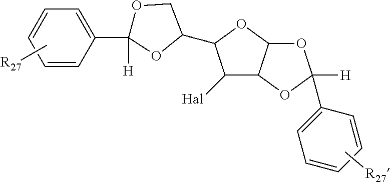 | Wherein R₂₇ and R₂₇′ have the same definitions as R₇, R₇′ in compound of formula Ig and wherein Hal is selected from the group consisting of fluoro, chloro, bromo and iodo |
| Compound of formula XVIc | 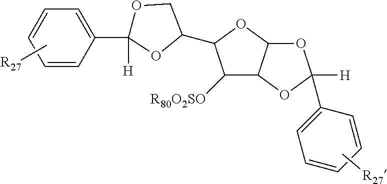 | Wherein R₂₇ and R₂₇′ have the same definitions as R₇, R₇′ in compound of formula Ig and wherein R₈₀ is methyl or p- methylphenyl |
| Compound of formula XVId | 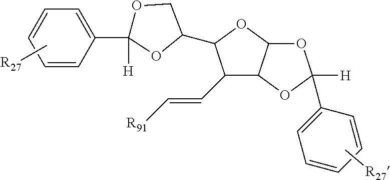 | Wherein R₂₇ and R₂₇′ have the same definitions as R₇, R₇′ in compound of formula Ig and wherein R₉₁ is a linear or branched C₁-C₁₈ alkyl, thus resulting in an alkenyl |
| Compound of formula XVIe | 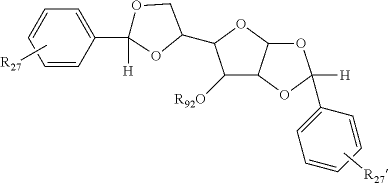 | Wherein R₂₇ and R₂₇′ have the same definitions as R₇, R₇′ in compound of formula Ig and wherein R₉₂ is a linear or branched C₁-C₁₈ alkyl, thus resulting in a linear or branched alkoxy |
| Compound of formula XVIf | 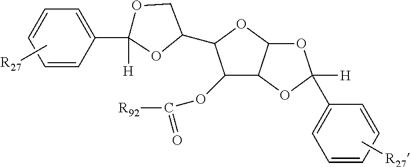 | Wherein R₂₇ and R₂₇′ have the same definitions as R₇, R₇′ in compound of formula Ig and wherein R₉₂ is a linear or branched C₁-C₁₈ alkyl, thus resulting in a linear or branched alkanoyloxy group |
| Compound of formula XIVg | 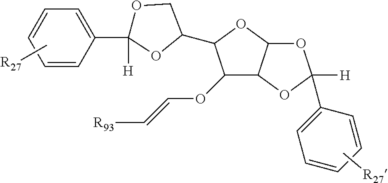 | Wherein R₂₇ and R₂₇′ have the same definitions as R₇, R₇′ in compound of formula Ig and wherein R₉₃ is a linear or branched C₁-C₁₈ alkyl, thus resulting in a linear or branched alkenyloxy |

In another embodiment of the present invention, the compound of formula (III) is a compound of formula (XVII),

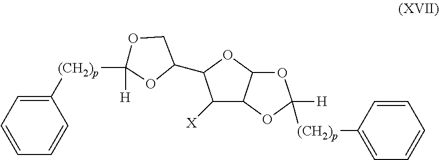

wherein X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, a linear or branched C₁ to C₉ alkylcarbonyl, and and p is a number between 1 and 6, and in particular a compound of formula XVIIa to XVIIg:

| | | |
|---|---|---|
| Compound of formula XVIIa | 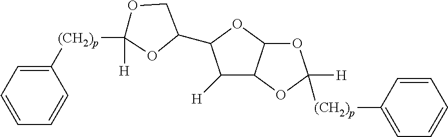 | Wherein p is a number between 1 and 6 |
| Compound of formula XVIIb | 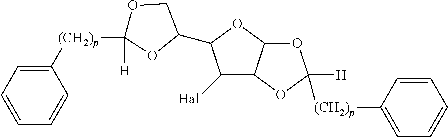 | Wherein p is a number between 1 and 6 and wherein Hal is selected from the group consisting of fluoro, chloro, bromo and iodo |
| Compound of formula XVIIc | 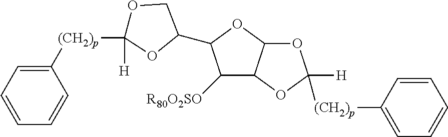 | Wherein p is a number between 1 and 6 and wherein R₈₀ is methyl or p- methylphenyl |
| Compound of formula XVIId | 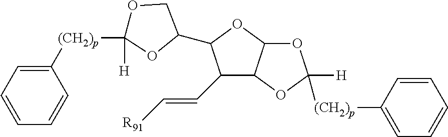 | Wherein p is a number between 1 and 6 and wherein R₉₁ is a linear or branched C₁-C₁₈ alkyl, thus resulting in an alkenyl |
| Compound of formula XVIIe | 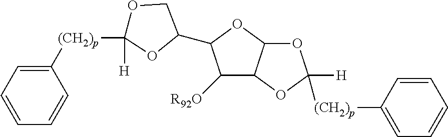 | Wherein p is a number between 1 and 6 and wherein R₉₂ is a linear or branched C₁-C₁₈ alkyl, thus resulting in a linear or branched alkoxy |
| Compound of formula XVIIf | 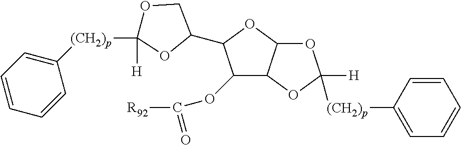 | Wherein p is a number between 1 and 6 and wherein R₉₂ is a linear or branched C₁-C₁₈ alkyl, resulting in a linear or branched alkanoyloxy group |
| Compound of formula XVIIg | 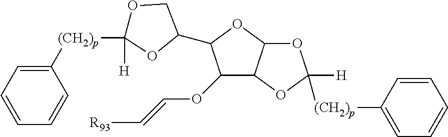 | Wherein p is a number between 1 and 6 and wherein R₉₃ is a linear or branched C₁-C₁₈ alkyl, thus resulting in a linear or branched alkenyloxy |

In another embodiment of the present invention, the compound of formula (IV) is a compound of formula (XX),

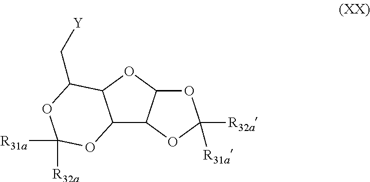

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is hydrogen, or a linear or branched C₁ to C₁₈ alkyl, a linear or branched C₁ to C₉ alkylcarbonyl, and R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia, in particular a compound of formula XXa to XXi:

| | | |
|---|---|---|
| Compound of formula XXa | 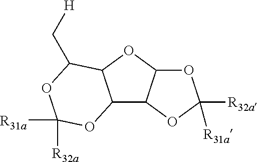 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia |
| Compound of formula XXb | 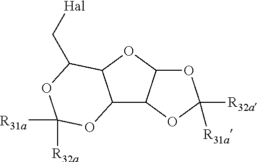 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein Hal is selected from the group consisting of fluoro, chloro, bromo and iodo |
| Compound of formula XXc | 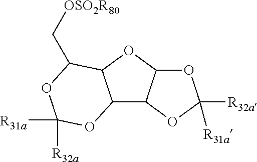 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₈₀ is methyl or p-methylphenyl |
| Compound of formula XXd | 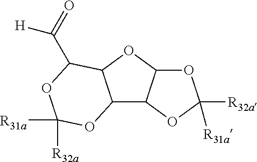 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia |
| Compound of formula XXe | 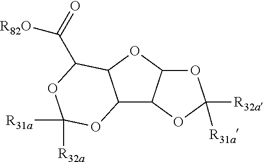 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₈₂ is a linear C₁-C₅ alkyl |
| Compound of formula XXf₁ | 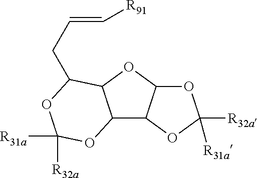 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₉₁ is a linear or branched C₁-C₁₈ alkyl, thus resulting in an alkenyl |
| Compound of formula XXf₂ | 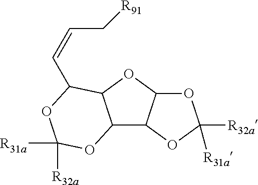 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₉₁ is a linear or branched C₁-C₁₈ alkyl, thus resulting in an alkenyl |
| Compound of formula XXg | 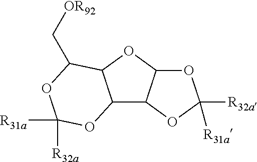 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₉₂ is a linear or branched C₁-C₁₈ alkyl, thus resulting in a linear or branched alkoxy |
| Compound of formula XXh | 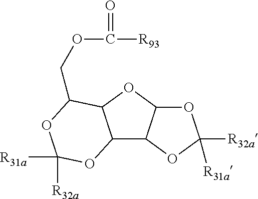 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₉₃ is a linear or branched C₁-C₁₈ alkyl, resulting in a linear or branched alkanoyloxy group |
| Compound of formula XXi | 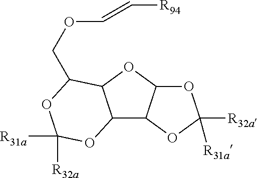 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₉₄ is a linear or branched C₁-C₁₈ alkyl, thus resulting in a linear or branched alkenyloxy |

In another embodiment of the present invention, the compound of formula (IV) is a compound of formula (XXI),

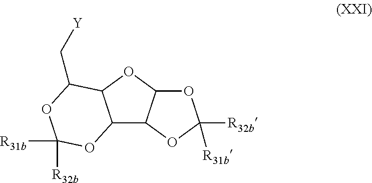

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and R_{31b}, R_{31b}′, R_{32b} and R_{32b}′ have the same definitions as R_{1b}, R_{1b}′, R_{2b} and R_{2b}′ in compound of formula Ib, in particular a compound of formula XXIa to XXIi:

| | | |
|---|---|---|
| Compound of formula XXIa | 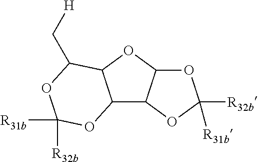 | Wherein R_{31b}, R_{31b}′, R_{32b} and R_{32b}′ have the same definitions as R_{1b}, R_{1b}′, R_{2b} and R_{2b}′ in compound of formula Ib |
| Compound of formula XXIb | 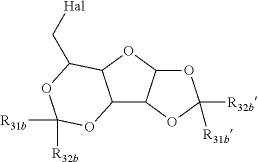 | Wherein R_{31b}, R_{31b}′, R_{32b} and R_{32b}′ have the same definitions as R_{1b}, R_{1b}′, R_{2b} and R_{2b}′ in compound of formula Ib and wherein Hal is selected from the group consisting of fluoro, chloro, bromo and iodo |
| Compound of formula XXIc | 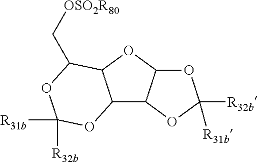 | Wherein R_{31b}, R_{31b}′, R_{32b} and R_{32b}′ have the same definitions as R_{1b}, R_{1b}′, R_{2b} and R_{2b}′ in compound of formula Ib and wherein R₈₀ is methyl or p-methylphenyl |
| Compound of formula XXIf₁ | 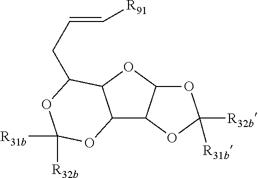 | Wherein R_{31b}, R_{31b}′, R_{32b} and R_{32b}′ have the same definitions as R_{1b}, R_{1b}′, R_{2b} and R_{2b}′ in compound of formula Ib and wherein R₉₁ is a linear or branched C₁-C₁₈ alkyl, thus resulting in an alkenyl |
| Compound of formula XXIf₂ | 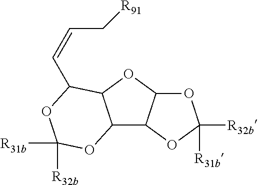 | Wherein R_{31b}, R_{31b}′, R_{32b} and R_{32b}′ have the same definitions as R_{1b}, R_{1b}′, R_{2b} and R_{2b}′ in compound of formula Ib and wherein R₉₁ is a linear or branched C₁-C₁₈ alkyl, thus resulting in an alkenyl |
| Compound of formula XXIg | 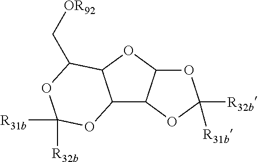 | Wherein R_{31b}, R_{31b}′, R_{32b} and R_{32b}′ have the same definitions as R_{1b}, R_{1b}′, R_{2b} and R_{2b}′ in compound of formula Ib and wherein R₉₂ is a linear or branched C₁-C₁₈ alkyl, thus resulting in a linear or branched alkoxy |
| Compound of formula XXIh | 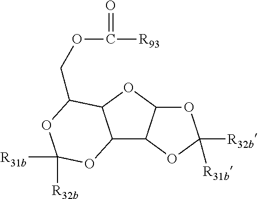 | Wherein R_{31b}, R_{31b}′, R_{32b} and R_{32b}′ have the same definitions as R_{1b}, R_{1b}′, R_{2b} and R_{2b}′ in compound of formula Ib and wherein R₉₃ is a linear or branched C₁-C₁₈ alkyl, resulting in a linear or branched alkanoyloxy group |
| Compound of formula XXIi | 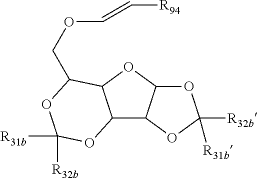 | Wherein R_{31b}, R_{31b}′, R_{32b} and R_{32b}′ have the same definitions as R_{1b}, R_{1b}′, R_{2b} and R_{2b}′ in compound of formula Ib and wherein R₉₄ is a linear or branched C₁-C₁₈ alkyl, thus resulting in a linear or branched alkenyloxy |

In another embodiment of the present invention, the compound of formula (IV) is a compound of formula (XXII),

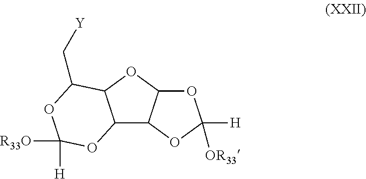

wherein Y is selected from the group consisting of hydrogen, fluoro, chloro, bromo, iodo, a linear or branched C₁ to C₁₈ alkyl, C₁ to C₁₈ alkenyl, a linear or branched C₂ to C₁₈ alkenyl, a sulfonate and OR₅₀, wherein R₅₀ is selected from the group consisting of hydrogen, a linear or branched C₁ to C₁₈ alkyl, a linear or branched C₁ to C₉ alkylcarbonyl, and R₃₃ and R₃₃′ have the same definitions as R₃ and R₃′ in compound of formula Ic, in particular a compound of formula XXIIa to XXIIi:

| | | |
|---|---|---|
| Compound of formula XXIIa | 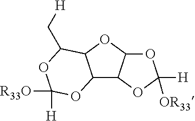 | Wherein R₃₃ and R₃₃′ have the same definitions as R₃, and R₃′ in compound of formula Ic |
| Compound of formula XXIIb | 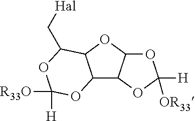 | Wherein R₃₃ and R₃₃′ have the same definitions as R₃, and R₃′ in compound of formula Ic and wherein Hal is selected from the group consisting of fluoro, |
| | | chloro, bromo and iodo |
| Compound of formula XXIIc | 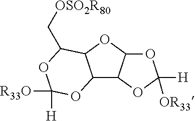 | Wherein R₃₃ and R₃₃′ have the same definitions as R₃, and R₃′ in compound of formula Ic and wherein Reo is methyl or p-methylphenyl |
| Compound of formula XXIIf1 | 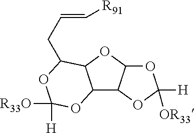 | Wherein R₃₃ and R₃₃′ have the same definitions as R₃, and R₃′ in compound of formula Ic and wherein R₉₁ is a linear or branched C₁-C₁₈ alkyl, thus |
| | | resulting in an |
| | | alkenyl |
| Compound of formula XXIIf2 | 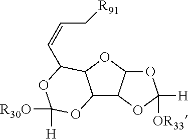 | Wherein R₃₃ and R₃₃′ have the same definitions as R₃, and R₃′ in compound of formula Ic and wherein R₉₁ is a linear or branched C₁-C₁₈ alkyl, thus resulting in an |
| | | alkenyl |
| Compound of formula XXIIg | 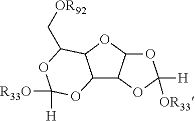 | Wherein R₃₃ and R₃₃′ have the same definitions as R₃, and R₃′ in compound of formula Ic and wherein R₉₂ is a linear or branched C₁₋C₁₈ alkyl, thus |
| | | resulting in a |
| | | linear or branched |
| | | alkoxy |
| Compound of formula XXIIh | 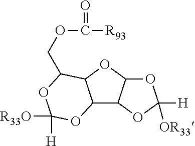 | Wherein R₃₃ and R₃₃′ have the same definitions as R₃, and R₃′ in compound of formula Ic and wherein R₉₃ is a linear or branched C₁-C₁₈ alkyl, resulting in a linear or branched |
| | | alkanoyloxy group |
| Compound of formula XXIIi | 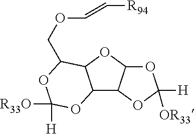 | Wherein R₃₃ and R₃₃′ have the same definitions as R₃, and R₃′ in compound of formula Ic and wherein R₉₄ is a linear or branched C₁-C₁₈ alkyl, thus |
| | | resulting in a |
| | | linear or branched |
| | | alkenyloxy |

In another embodiment of the present invention, the compound of formula (IV) is a compound of formula (XXIII),

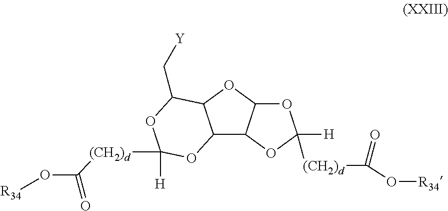

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀ wherein R₅₀ is hydrogen, or a linear or branched C₁ to C₁₈ alkyl, a linear or branched C₁ to C₉ alkylcarbonyl, and and R₃₄ and R₃₄′ have the same definitions as R₄ and R₄′ in compound of formula Id, and d is 0, 1, or 2, in particular a compound of formula XXIIIa to XXIIIi:

| | | |
|---|---|---|
| Compound of formula XXIIIa | 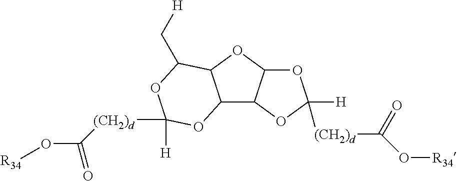 | Wherein R₃₄ and R₃₄′ have the same definitions as R₄, and R₄′ in compound of formula Id and d is 0, 1 or 2 |
| Compound of formula XXIIIb | 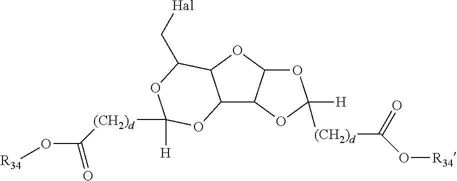 | Wherein R₃₄ and R₃₄′ have the same definitions as R₄, and R₄′ in compound of formula Id, d is 0, 1 or 2 and wherein Hal is selected from the group consisting of fluoro, chloro, bromo and iodo |
| Compound of formula XXIIIc | 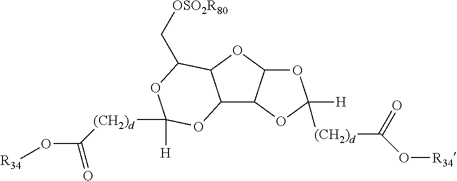 | Wherein R₃₄ and R₃₄′ have the same definitions as R₄, and R₄′ in compound of formula Id, d is 0, 1 or 2 and wherein R₈₀ is methyl or p- methylphenyl |
| Compound of formula XXIIIf₁ | 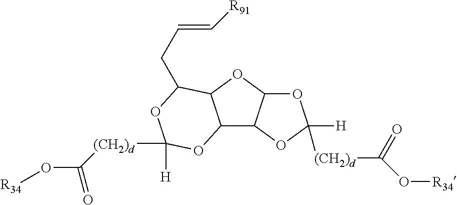 | Wherein R₃₄ and R₃₄′ have the same definitions as R₄, and R₄′ in compound of formula Id, d is 0, 1 or 2 and wherein R₉₁ is a linear or branched C₁-C₁₀ alkyl, thus resulting in an alkenyl |
| Compound of formula XXIIIf₂ | 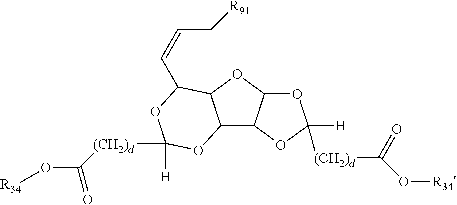 | Wherein R₃₄ and R₃₄′ have the same definitions as R₄, and R₄′ in compound of formula Id, d is 0, 1 or 2 and wherein R₉₁ is a linear or branched C₁-C₁₈ alkyl, thus resulting in an alkenyl |
| Compound of formula XXIIIg | 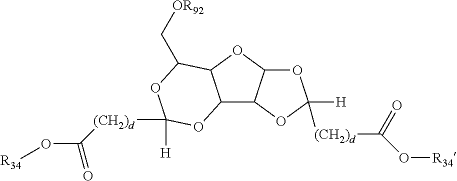 | Wherein R₃₄ and R₃₄′ have the same definitions as R₄, and R₄′ in compound of formula Id, d is 0, 1 or 2 and wherein R₉₂ is a linear or branched C₁-C₁₈ alkyl, thus resulting in a linear or branched alkoxy |
| Compound of formula XXIIIh | 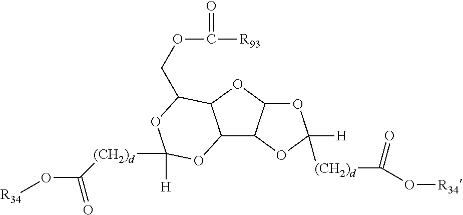 | Wherein R₃₄ and R₃₄′ have the same definitions as R₄, and R₄′ in compound of formula Id, d is 0, 1 or 2 and wherein R₉₃ is a linear or branched C₁-C₁₈ alkyl, resulting in a linear or branched alkanoyloxy group |
| Compound of formula XXIIIi | 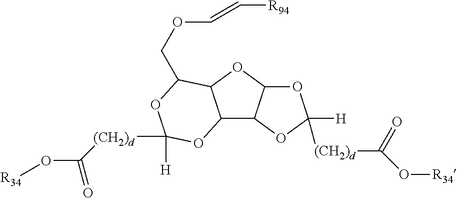 | Wherein R₃₄ and R₃₄′ have the same definitions as R₄, and R₄′ in compound of formula Id, d is 0, 1 or 2 and wherein R₉₄ is a linear or branched C₁-C₁₈ alkyl, thus resulting in a linear or branched alkenyloxy |

In another embodiment of the present invention, the compound of formula (IV) is a compound of formula (XXIV),

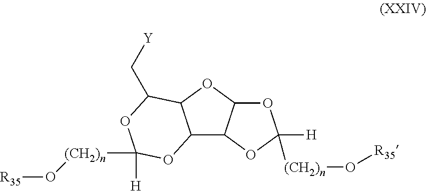

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀ wherein R₅₀ is selected from the group consisting of hydrogen, or a linear or branched C₁ to C₁₈ alkyl, a linear or branched C₁ to C₉ alkylcarbonyl, and R₃₅ and R₃₅′ have the same definitions as R₅ and R₅′ in compound of formula Ie, and n is a number between 1 to 9, in particular a compound of formula XXIVa to XXIVi:

| | | |
|---|---|---|
| Compound of formula XXIVa | 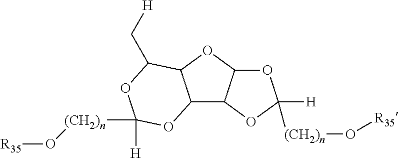 | Wherein R₃₅ and R₃₅′ have the same definitions as R₅, and R₅′ in compound of formula Ie and n is a number between 1 and 9 |
| Compound of formula XXIVb | 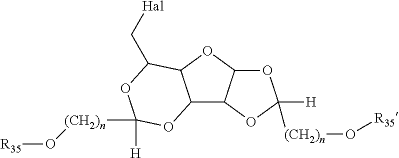 | Wherein R₃₅ and R₃₅′ have the same definitions as R₅, and R₅′ in compound of formula Ie, n is a number between 1 and 9 and wherein Hal is selected from the group consisting of fluoro, chloro, bromo and iodo |
| Compound of formula XXIVc | 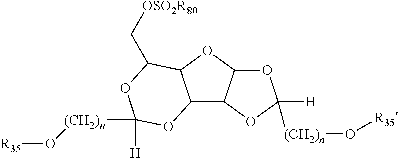 | Wherein R₃₅ and R₃₅′ have the same definitions as R₅, and R₅′ in compound of formula Ie, n is a number between 1 and 9 and wherein R₈₀ is methyl or p- methylphenyl |
| Compound of formula XXIVf₁ | 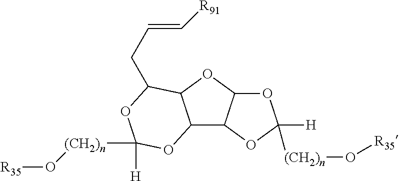 | Wherein R₃₅ and R₃₅′ have the same definitions as R₅, and R₅′ in compound of formula Ie, n is a number between 1 and 9 and wherein R₉₁ is a linear or branched C₁- C₁₈ alkyl, thus resulting in an alkenyl |
| Compound of formula XXIVf₂ | 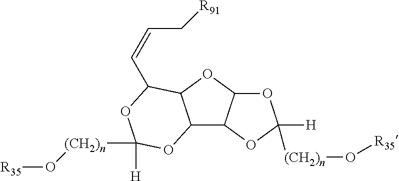 | Wherein R₃₅ and R₃₅′ have the same definitions as R₅, and R₅′ in compound of formula Ie, n is a number between 1 and 9 and wherein R₉₁ is a linear or branched C₁- C₁₈ alkyl, thus resulting in an alkenyl |
| Compound of formula XXIVg | 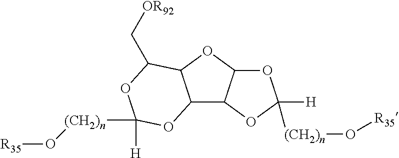 | Wherein R₃₅ and R₃₅′ have the same definitions as R₅, and R₅′ in compound of formula Ie, n is a number between 1 and 9 and wherein R₉₂ is a linear or branched C₁- C₁₈ alkyl, thus resulting in a linear or branched alkoxy |
| Compound of formula XXIVh | 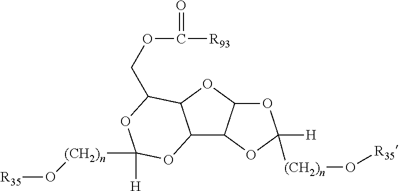 | Wherein R₃₅ and R₃₅′ have the same definitions as R₅, and R₅′ in compound of formula Ie, n is a number between 1 and 9 and wherein R₉₃ is a linear or branched C₁- C₁₈ alkyl, resulting in a linear or branched alkanoyloxy group |
| Compound of formula XXIVi | 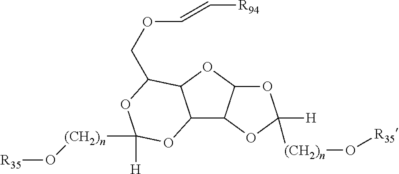 | Wherein R₃₅ and R₃₅′ have the same definitions as R₅, and R₅′ in compound of formula Ie, n is a number between 1 and 9 and wherein R₉₄ is a linear or branched C₁- C₁₈ alkyl, thus resulting in a linear or branched alkenyloxy |

In another embodiment of the present invention, the compound of formula (IV) is a compound of formula (XXV),

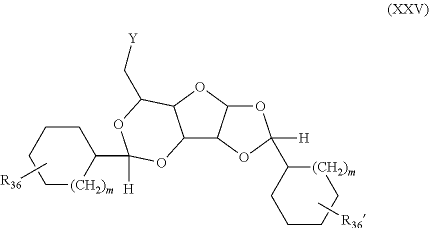

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀ wherein R₅₀ is selected from the group consisting of hydrogen, or a linear or branched C₁ to C₁₈ alkyl, a linear or branched C₁ to C₉ alkylcarbonyl, and R₃₆ and R₃₆′ have the same definitions as R₆ and R₆′ in compound of formula If, and a is a number between 0 and 6 in particular a compound of formula XXVa to XXVi:

| | | |
|---|---|---|
| Compound of formula XXVa | 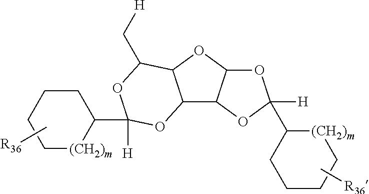 | Wherein R₃₆ and R₃₆′ have the same definitions as R₆, and R₆′ in compound of formula If and m is a number between 0 and 6 |
| Compound of formula XXVb | 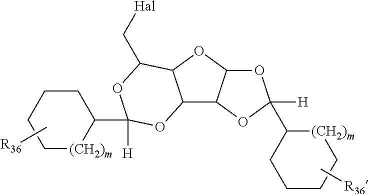 | Wherein R₃₆ and R₃₆′ have the same definitions as R₆, and R₆′ in compound of formula If, m is a number between 0 and 6 and wherein Hal is selected from the group consisting of fluoro, chloro, bromo and iodo |
| Compound of formula XXVc | 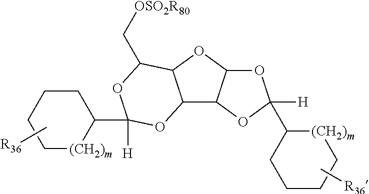 | Wherein R₃₆ and R₃₆′ have the same definitions as R₆, and R₆′ in compound of formula If, m is a number between 0 and 6 and wherein R₈₀ is methyl or p- methylphenyl |
| Compound of formula XXVf1 | 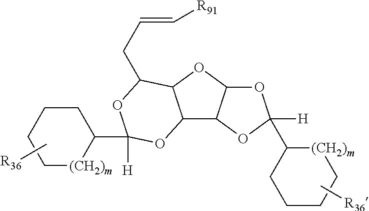 | Wherein R₃₆ and R₃₆′ have the same definitions as R₆, and R₆′ in compound of formula If, m is a number between 0 and 6 and wherein R₉₁ is a linear or branched C₁- C₁₈ alkyl, thus resulting in an alkenyl |
| Compound of formula XXVf2 | 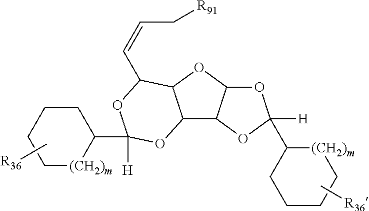 | Wherein R₃₆ and R₃₆′ have the same definitions as R₆, and R₆′ in compound of formula If, m is a number between 0 and 6 and wherein R₉₁ is a linear or branched C₁- C₁₈ alkyl, thus resulting in an alkenyl |
| Compound of formula XXVg | 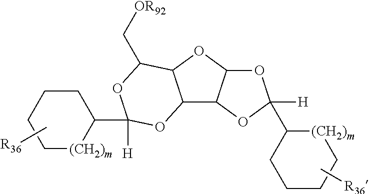 | Wherein R₃₆ and R₃₆′ have the same definitions as R₆, and R₆′ in compound of formula If, m is a number between 0 and 6 and wherein R₉₂ is a linear or branched C₁- C₁₈ alkyl, thus resulting in a linear or branched alkoxy |
| Compound of formula XXVh | 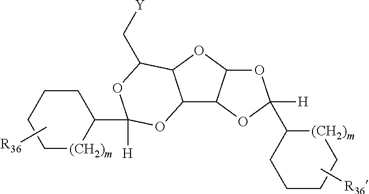 | Wherein R₃₆ and R₃₆′ have the same definitions as R₆, and R₆′ in compound of formula If, m is a number between 0 and 6 and wherein R₉₃ is a linear or branched C₁- C₁₈ alkyl, resulting in a linear or branched alkanoyloxy group |
| Compound of formula XXVi | 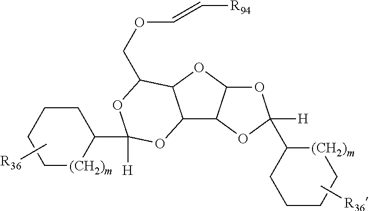 | Wherein R₃₆ and R₃₆′ have the same definitions as R₆, and R₆′ in compound of formula If, m is a number between 0 and 6 and wherein R₉₄ is a linear or branched C₁- C₁₈ alkyl, thus resulting in a linear or branched alkenyloxy |

In another embodiment of the present invention, the compound of formula (IV) is a compound of formula (XXVI),

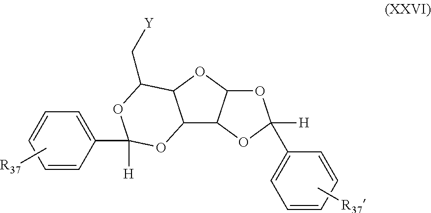

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀ wherein R₅₀ is selected from the group consisting of hydrogen, or a linear or branched C₁ to C₁₈ alkyl, a linear or branched C₁ to C alkylcarbonyl, and R₃₇ and R₃₇′ have the same definitions as R₇ and R₇′ in compound of 5 formula Ig, in particular a compound of formula XXVIa to XXVIi:

| | | |
|---|---|---|
| Compound of formula XXVIa | 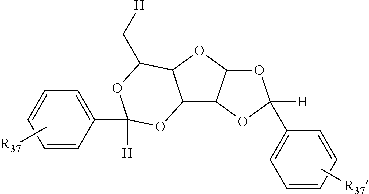 | Wherein R₃₇ and R₃₇′ have the same definitions as R₇, and R₇′ in compound of formula Ig |
| Compound of formula XXVIb | 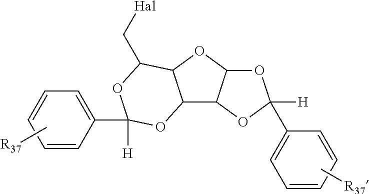 | Wherein R₃₇ and R₃₇′ have the same definitions as R₇, and R₇′ in compound of formula Ig and wherein Hal is selected from the group consisting of fluoro, chloro, bromo and iodo |
| Compound of formula XXVIc | 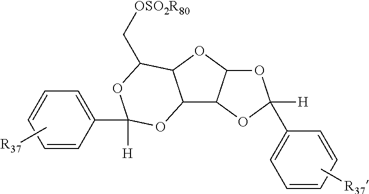 | Wherein R₃₇ and R₃₇′ have the same definitions as R₇, and R₇′ in compound of formula Ig and wherein Reo is methyl or p- methylphenyl |
| Compound of formula XXVIf₁ | 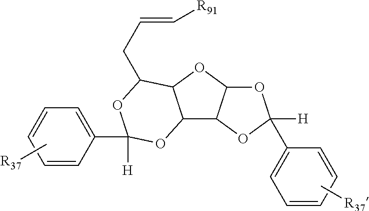 | Wherein R₃₇ and R₃₇′ have the same definitions as R₇, and R₇′ in compound of formula Ig and wherein R₉₁ is a linear or branched C₁-C₁₈ alkyl, thus resulting in an alkenyl |
| Compound of formula XXVIf₂ | 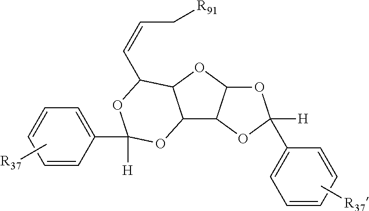 | Wherein R₃₇ and R₃₇′ have the same definitions as R₇, and R₇′ in compound of formula Ig and wherein R₉₁ is a linear or branched C₁-C₁₈ alkyl, thus resulting in an alkenyl |
| Compound of formula XXVIg | 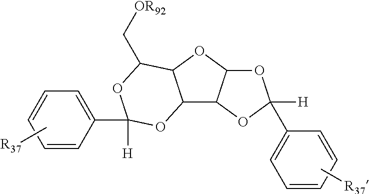 | Wherein R₃₇ and R₃₇′ have the same definitions as R₇, and R₇′ in compound of formula Ig and wherein R₉₂ is a linear or branched C₁-C₁₈ alkyl, thus resulting in a linear or branched alkoxy |
| Compound of formula XXVIh | 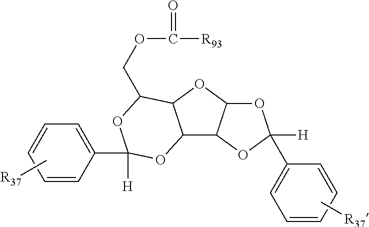 | Wherein R₃₇ and R₃₇′ have the same definitions as R₇, and R₇′ in compound of formula Ig and wherein R₉₃ is a linear or branched C₁-C₁₈ alkyl, resulting in a linear or branched alkanoyloxy group |
| Compound of formula XXVIi | 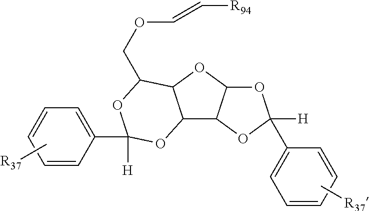 | Wherein R₃₇ and R₃₇′ have the same definitions as R₇, and R₇′ in compound of formula Ig and wherein R₉₄ is a linear or branched C₁-C₁₈ alkyl, thus resulting in a linear or branched alkenyloxy |

In another embodiment of the present invention, the compound of formula (IV) is a compound of formula (XXVII),

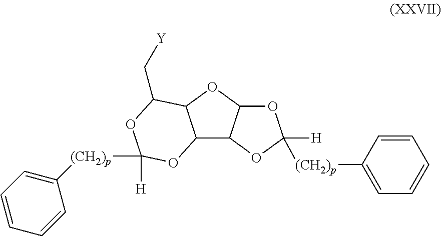

wherein Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀ wherein R₅₀ is selected from the group consisting of hydrogen, or a linear or branched C₁ to C₁₈ alkyl, a linear or branched C₁ to C₉ alkylcarbonyl, and p is a number between 1 and 6, in particular a compound of formula XXVIIa to XXVIIi:

| | | |
|---|---|---|
| Compound of formula XXVIIa | 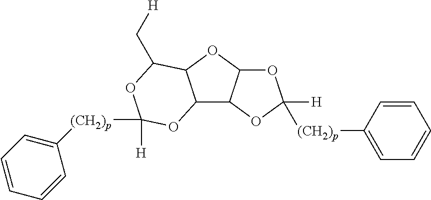 | Wherein p is a number between 1 and 6 |
| Compound of formula XXVIIb | 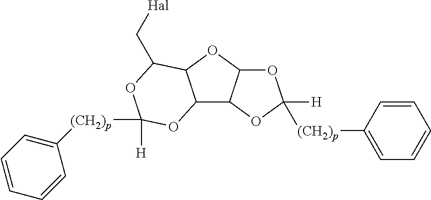 | Wherein p is a number between 1 and 6 and wherein Hal is selected from the group consisting of fluoro, chloro, bromo and iodo |
| Compound of formula XXVIIc | 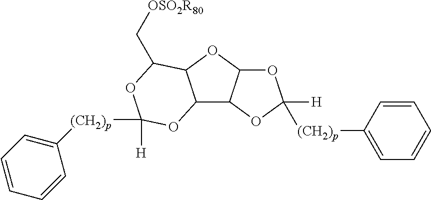 | Wherein p is a number between 1 and 6 and wherein R₈₀ is methyl or p-methylphenyl |
| Compound of formula XXVIIf₁ | 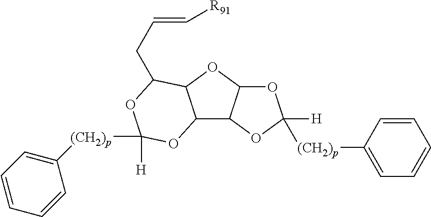 | Wherein p is a number between 1 and 6 and wherein R₉₁ is a linear or branched C₁-C₁₈ alkyl, thus resulting in an alkenyl |
| Compound of formula XXVIIf₂ | 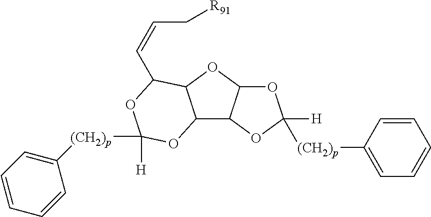 | Wherein p is a number between 1 and 6 and wherein R₉₁ is a linear or branched C₁-C₁₈ alkyl, thus resulting in an alkenyl |
| Compound of formula XXVIIg | 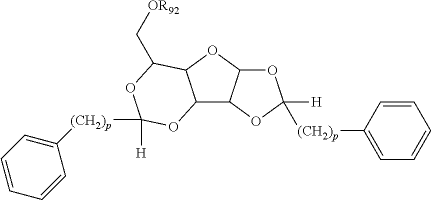 | Wherein p is a number between 1 and 6 and wherein R₉₂ is a linear or branched C₁-C₁₈ alkyl, thus resulting in a linear or branched alkoxy |
| Compound of formula XXVIIh | 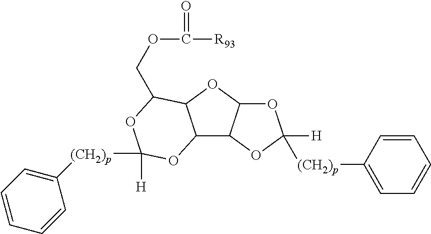 | Wherein p is a number between 1 and 6 and wherein R₉₃ is a linear or branched C₁-C₁₈ alkyl, resulting in a linear or branched alkanoyloxy group |
| Compound of formula XXVIIi | 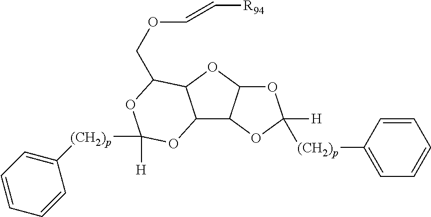 | Wherein p is a number between 1 and 6 and wherein R₉₄ is a linear or branched C₁-C₁₈ alkyl, thus resulting in a linear or branched alkenyloxy |

In another embodiment of the present invention, in the compound of formula (III) and (IV) R₅₀ is hydrogen or in compound (IV), Y is oxygen which forms together with the adjacent carbon atom carboxy group. Particularly preferred are the following compounds

| | | |
|---|---|---|
| Compound of formula Xh | 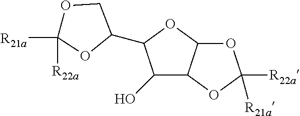 | Wherein R₂₁ₐ, R₂₁ₐ′, R₂₂ₐ and R₂₂ₐ′ have the same definitions as R₁ₐ, R₁ₐ′, R₂ₐ and R₂ₐ′ in compound of formula Ia |
| Compound of formula XIh | 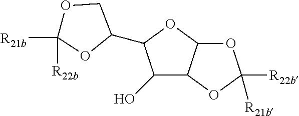 | Wherein R_{21b}, R_{21b}′, R_{22b} and R_{22b}′ have the same definitions as R_{1b}, R_{1b}′, R_{2b} and R_{2b}′ in compound of formula Ib |
| Compound of formula XIIh | 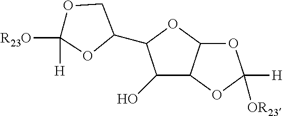 | Wherein R₂₃ and R₂₃′ have the same definitions as R₃, R₃′ in compound of formula Ic |
| Compound of formula XIIIh | 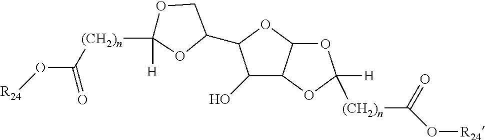 | Wherein R₂₄ and R₂₄′ have the same definitions as R₄, R₄′ in compound of formula Id and d is 0, 1 or 2 |
| Compound of formula XIVh | 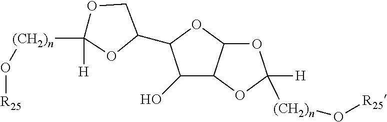 | Wherein R₂₅ and R₂₅′ have the same definitions as R₅, R₅′ in compound of formula Ie and n is 1 or 2 |
| Compound of formula XVh | 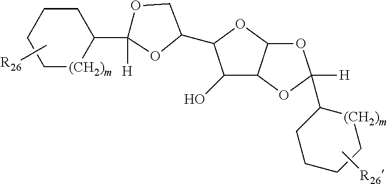 | Wherein R₂₆ and R₂₆′ have the same definitions as R₆, R₆′ in compound of formula If and m is between 0 and 6 |
| Compound of formula XVIh | 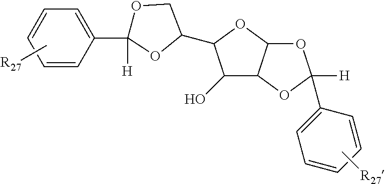 | Wherein R₂₇ and R₂₇′ have the same definitions as R₇, R₇′ in compound of formula Ig |
| Compound of formula XVIIh | 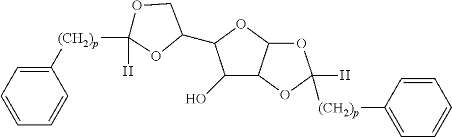 | Wherein p is a number between 1 and 6 |
| Compound of formula XXk | 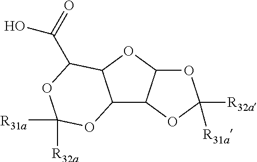 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia |
| Compound of formula XXm | 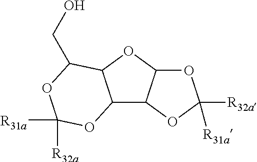 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia |
| Compound of formula XXIk | 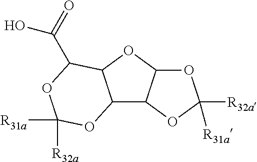 | Wherein R_{31b}, R_{31b}′, R_{32b} and R_{32b}′ have the same definitions as R_{1b}, R_{1b}′, R_{2b} and R_{2b}′ in compound of formula Ib |
| Compound of formula XXIm | 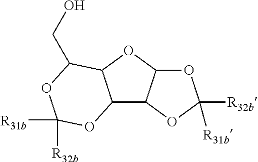 | Wherein R_{31b}, R_{31b}′, R_{32b} and R_{32b}′ have the same definitions as R_{1b}, R_{1b}′, R_{2b} and R_{2b}′ in compound of formula Ib |
| Compound of formula XXIIk | 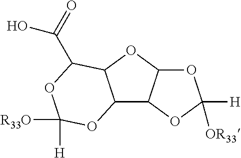 | Wherein R₃₃ and R₃₃′ have the same definitions as R₃, and R₃′ in compound of formula Ic |
| Compound of formula XXIIm | 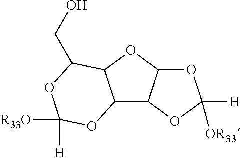 | Wherein R₃₃ and R₃₃′ have the same definitions as R₃, and R₃′ in compound of formula Ic |
| Compound of formula XXIIIk | 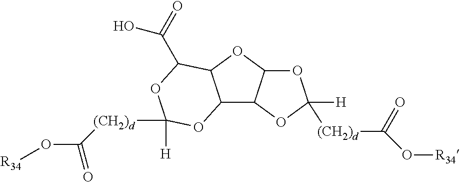 | Wherein R₃₄ and R₃₄′ have the same definitions as R₄, and R₄′ in compound of formula Id and d is 0, 1 or 2 |
| Compound of formula XXIIIm | 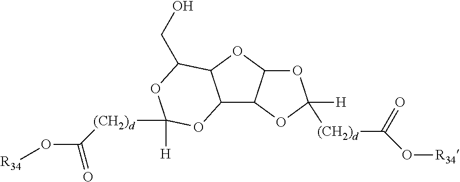 | Wherein R₃₄ and R₃₄′ have the same definitions as R₄, and R₄′ in compound of formula Id and d is 0, 1 or 2 |
| Compound of formula XXIVk | 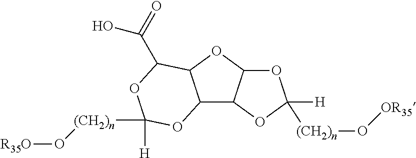 | Wherein R₃₅ and R₃₅′ have the same definitions as R₅, and R₅′ in compound of formula Ie and n is a number between 1 to 9 |
| Compound of formula XXIVm | 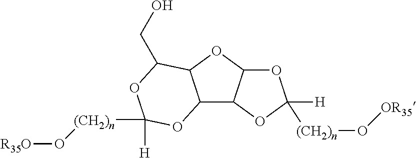 | Wherein R₃₅ and R₃₅′ have the same definitions as R₅, and R₅′ in compound of formula Ie and n is a number between 1 to 9 and |
| Compound of formula XXVk | 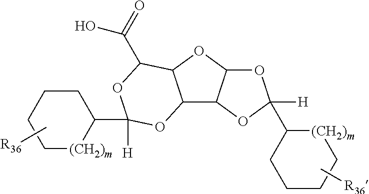 | Wherein R₃₆ and R₃₆′ have the same definitions as R₆, and R₆′ in compound of formula If and m is a number between 0 to 6 |
| Compound of formula XXVm | 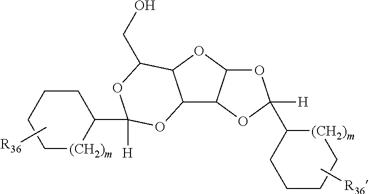 | Wherein R₃₆ and R₃₆′ have the same definitions as R₆, and R₆′ in compound of formula If and m is a number between 0 to 6 |
| Compound of formula XXVIk | 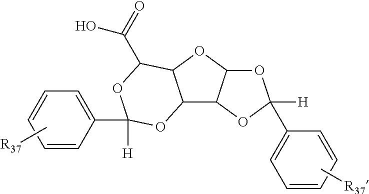 | Wherein R₃₇ and R₃₇′ have the same definitions as R₇, and R₇′ in compound of formula Ig |
| Compound of formula XXVIm | 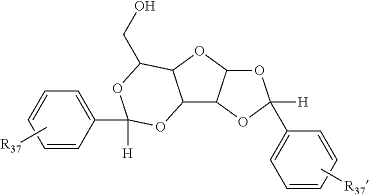 | Wherein R₃₇ and R₃₇′ have the same definitions as R₇, and R₇′ in compound of formula Ig |
| Compound of formula XXVIIk | 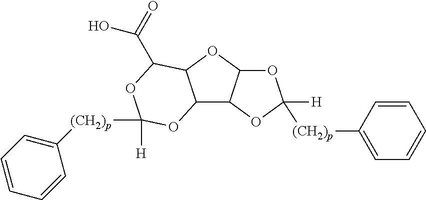 | Wherein p is a number between 1 and 6 |
| Compound of formula XXVIIm | 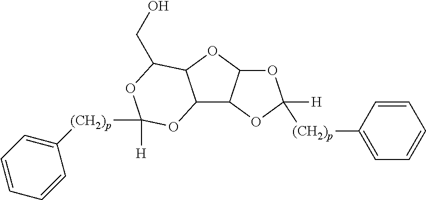 | Wherein p is a number between 1 and 6 |

In another embodiment of the present invention, in the compound of formula (V) R₆₀ is selected from the group consisting of hydrogen, hydroxy and C₁ to C₁₈ alkoxy,

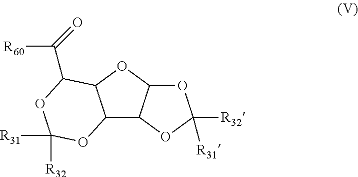

wherein and R₃₁ₐ, R₃₁ₐ′, R₃₂ₐ and R₃₂′ have the same definitions as R₁ₐ, R₁ₐ′, R₂ₐ and R₂ₐ′ in compound of formula (Ia). Particularly preferred are the following compounds

| | | |
|---|---|---|
| Compound of formula Vd | 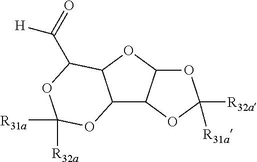 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia |
| Compound of formula Ve | 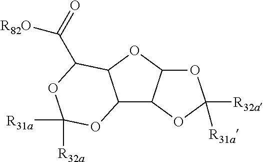 | Wherein R₃₁ₐ, R₃₁ₐ′, R₃₂ₐ and R₃₂ₐ′ have the same definitions as R₁ₐ, R₁ₐ′, R₂ₐ and R₂ₐ′ in compound of formula Ia and wherein R₈₂ is a linear C₁-C₅ alkyl |
| Compound of formula XXId | 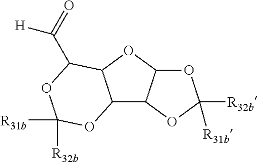 | Wherein R_{31b}, R_{31b}′, R_{32b} and R_{32b}′ have the same definitions as R_{1b}, R_{1b}′, R_{2b} and R_{2b}′ in compound of formula Ib |
| Compound of formula XXIe | 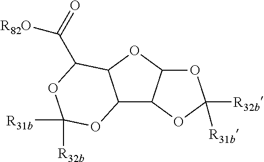 | Wherein R_{31b}, R_{31b}′, R_{32b} and R_{32b}′ have the same definitions as R_{1b}, R_{1b}′, R_{2b} and R_{2b}′ in compound of formula Ib and wherein R₈₂ is a linear C₁-C₅ alkyl |
| Compound of formula XXIId | 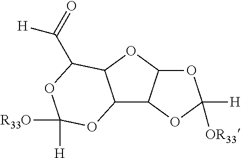 | Wherein R₃₃ and R₃₃′ have the same definitions as R₃, and R₃′ in compound of formula Ic |
| Compound of formula XXIIe | 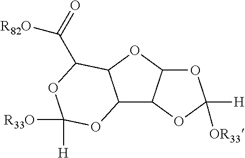 | Wherein R₃₃ and R₃₃′ have the same definitions as R₃, and R₃′ in compound of formula Ic and wherein R₈₂ is a linear C₁-C₅ alkyl |
| Compound of formula XXIIId | 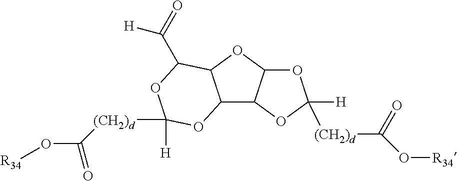 | Wherein R₃₄ and R₃₄′ have the same definitions as R₄, and R₄′ in compound of formula Id and d is 0, 1 or 2 |
| Compound of formula XXIIIe | 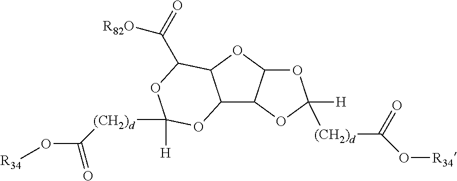 | Wherein R₃₄ and R₃₄′ have the same definitions as R₄, and R₄′ in compound of formula Id, d is 0, 1 or 2 and wherein R₈₂ is a linear C₁-C₅ alkyl |
| Compound of formula XXIVd | 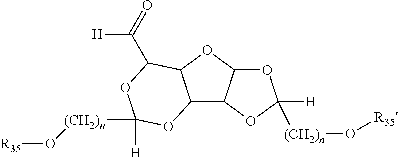 | Wherein R₃₅ and R₃₅′ have the same definitions as R₅, and R₅′ in compound of formula Ie and n is a number between 1 and 9 |
| Compound of formula XXIVe | 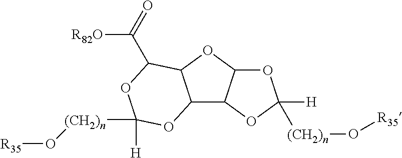 | Wherein R₃₅ and R₃₅′ have the same definitions as R₅, and R₅′ in compound of formula Ie, n is a number between 1 and 9 and wherein R₈₂ is a linear C₁-C₅ alkyl |
| Compound of formula XXVd | 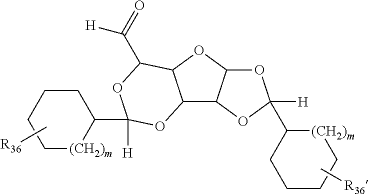 | Wherein R₃₆ and R₃₆′ have the same definitions as R₆, and R₆′ in compound of formula If and m is a number between 0 and 6 |
| Compound of formula XXVe | 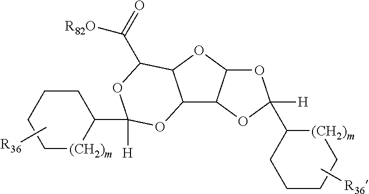 | Wherein R₃₆ and R₃₆′ have the same definitions as R₆, and R₆′ in compound of formula If, m is a number between 0 to 6 and wherein R₈₂ is a linear C₁-C₅ alkyl |
| Compound of formula XXVId | 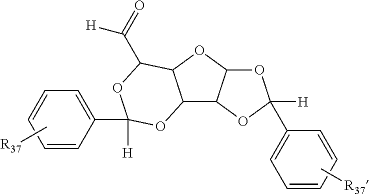 | Wherein R₃₇ and R₃₇′ have the same definitions as R₇, and R₇′ in compound of formula Ig |
| Compound of formula XXVIe | 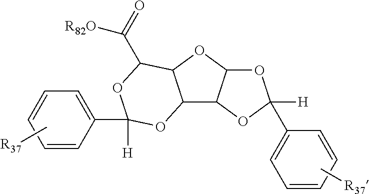 | Wherein R₃₇ and R₃₇′ have the same definitions as R₇, and R₇′ in compound of formula Ig and wherein R₈₂ is a linear C₁-C₅ alkyl |
| Compound of formula XXVIId | 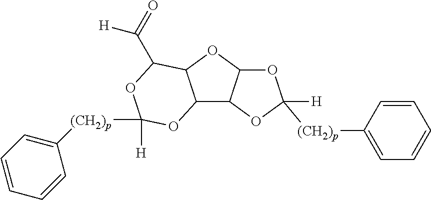 | Wherein p is a number between 1 and 6 |
| Compound of formula XXVIIe | 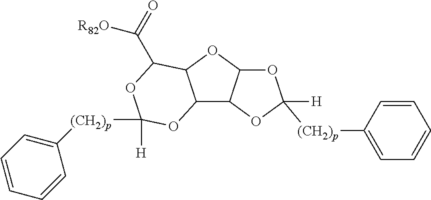 | Wherein p is a number between 1 and 6 and wherein R₈₂ is a linear C₁-C₅ alkyl |

In one embodiment, the compounds according to the present invention are used as solvent for a chemical reaction, preferably for an organic synthesis, in which at least one starting compound, in particular rearrangement reactions, such as Boulton-Katritzky rearrangement and Beckmann rearrangement, preferably at least two starting compounds are involved, and in particular chemical reactions, in which acid or basic conditions are used.

In another embodiment, the compounds according to the present invention are used as solvents for recrystallizations since they have high boiling points and do not react with the purified product. Preferred are compounds of formula (I) and (II), wherein R₁, R₁′, R₂ and R₂′ are all different from hydrogen as these compounds are solvents with a suitable melting point for a recrystallization. In contrast, diformyl xylose has a melting point of 48° C. which is not a suitable recrystallization solvent for many desired products.

In another embodiment, the compounds according to the present invention are used as solvents for extractions since they can solubilize a broad variety of possible target compounds. Furthermore, in case of the polar aprotic solvents they are almost immiscible with water.

The compounds according to the present invention are moderately stable in the presence of acids and bases and can therefore be used under these reaction conditions. In particular, compounds of the formulae (Ia), (Ie), (If), (Ig), (Ih), (Xa-Xg), (XIVa-XIVg), (XVa-XVg), (XVIa-XVIg), (XXa-XXg), (XXIVa-XXIVg), (XXVa-XXVg) and (XXVIa-XXVIg) are relatively stable and thus especially preferred in the presence of strong acids or strong bases. As used herein, the term “strong acid” means an acid that has a pKa less than 4. Strong acids include, for example, H₂SO₄, HCl and H₃PO₄. As used herein, the term “strong base” means a base that dissociate into metal ions and hydroxide ions in solution (e.g. NaOH, KOH).

Thus, according to a preferred embodiment, the solvent is used for a chemical reaction in the presence of an acid or a base, preferably a strong acid or strong base. Advantageously the chemical reaction takes place in the presence of an acid that has a pKa less than 4, e.g. H₂SO₄, HCl and H₃PO₄, or a base that dissociates into metal ions and hydroxide ions in solution, such as NaOH, KOH.

According to a preferred embodiment of the present invention the solvent is used for a chemical reaction, such as alkylation reaction, hydrogenation reaction, palladium-catalyzed cross-coupling reaction, or aldehyde-assisted biomass fractionation, e.g. biomass fractionation assisted by formaldehyde, propianaldehyde, butyraldehyde, isobutyraldehyde, or other aldehydes, in the presence of an acid or a base, preferably a strong acid.

In one embodiment R₁ and R₁′ are preferably hydrogen, a linear C₁ to C₁₀ alkyl, a linear C₂ to C₁₀ alkenyl, a linear C₁ to C₁₀ alkoxy, a linear C₁ to C₉ alkanoyloxy, a linear C₁ to C₉ alkoxycarbonyl or a linear C₁ to C₉ alkoxyalkyl, and R₂ and R₂′ are preferably both hydrogens. Such compounds can be easily obtained by reacting D-xylose with the corresponding aldehyde such as propanal, butanal, pentanal, hexanal, cyclohexanal, benzaldehyd, 4-methylbenzaldehyde.

In one embodiment in the compounds of formula (I) R₁ and R₁′ are preferably a branched C₁ to C₁₀ alkyl, a branched C₁ to C₉ alkoxyalkyl, and wherein R₂ and R₂′ are both hydrogen. Preferably the branching occurs in Ω-position, i.e. at the terminal positon of the alkyl chain. Most preferably R₁ and R₁′ are selected from the group consisting of isopropyl, isobutyl, tert-butyl, isopentyl, neopentyl and isohexyl. Such compounds can be easily obtained by reacting D-xylose with a branched aldehyde such as isobutyraldehyde, isovaleraldehyde, pivaldehyde, isooctanal, isodecanal, 7-tert-butoxyheptanal, preferably the aldehyde is selected from a group consisting of isobutyraldehyde, pivaldehyde and isovaleraldehyde.

Due to their excellent combination of solvent properties in terms of polarity, boiling point, melting point, viscosity and their poor miscibility with water the polar, aprotic solvents of the present invention can be used for a broad variety of chemical reaction. Especially good results could be obtained for reactions selected from the group consisting of the Heck reaction, alkylation reaction, hydrogenation reaction, Ullmann reaction or Ullmann coupling, Wurtz reaction, Grignard reaction, Gomberg-Bachmann reaction, Castro-Stephens coupling, Corey-House synthesis, Cassar reaction, Kumada coupling, Sonogashira coupling, Negishi coupling, Stille cross coupling, Suzuki reaction, Hiyama coupling, Buchwald-Hartwig reaction, Fukuyama coupling, Liebeskind-Srogl coupling, Solid-Phase Peptide Synthesis (SPPS), nucleophilic substitution, amidation, esterification, biginelli reaction, carbonyl addition, aza-Michael addition, Krapcho dealkoxycarbonylation, Boulton-Katritzky rearrangement and Beckmann rearrangement. Said name reactions are known to the skilled person.

Especially good results can be obtained when using the compounds according to the present invention as polar, aprotic solvent for an alkylation reaction and in particular for the Menshutkin reaction. Alkylation reactions are among the most prevailing transformations performed in medicinal chemistry and pharmaceutical industry and are known to be quite sensitive to solvent polarity. It could be shown that the compounds according to the present invention facilitate a charge separation at the transition state via favourable solute-solvent interactions, accelerating the whole process.

Thus, the compounds according to the present invention are outstanding alternatives for toxic solvents such as DMSO, DMF and NMP.

Furthermore, the polar aprotic solvents according to the present invention are particularly useful in a Heck/Mizoroki-Heck reaction. The Heck/Mizoroki-Heck reaction is a palladium-catalyzed cross-coupling reaction that is ubiquitous in the pharmaceutical industry and is routinely used for preparation of substituted alkenes from aryl halides. As shown in the examples, the compounds of the present invention have all the excellent properties to promote a Heck reaction.

It could be shown that the compounds according to the present invention are suitable solvents for hydrogenation reaction. As shown in the Examples, the compounds according to the present invention are suitable bio-based solvents for hydrogenation reactions, preferably in the presence of a catalyst, which is most preferably present in a concentration of between 5 to 15% by weight.

Aprotic polar solvents being used by the present invention or being produced by the method according to the present invention are especially preferred selected from group consisting of compounds 1 to 80:

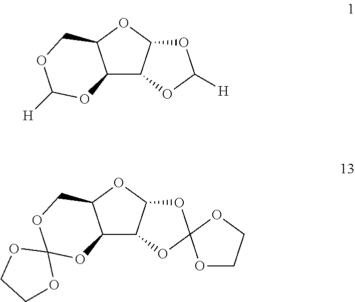 [SMILES: C1OC[C@H]2O[C@@H]3OCO[C@@H]3[C@H]2O1 ; C1COC2(O1)OC[C@H]1O[C@@H]3OC4(OCCO4)O[C@@H]3[C@H]1O2]

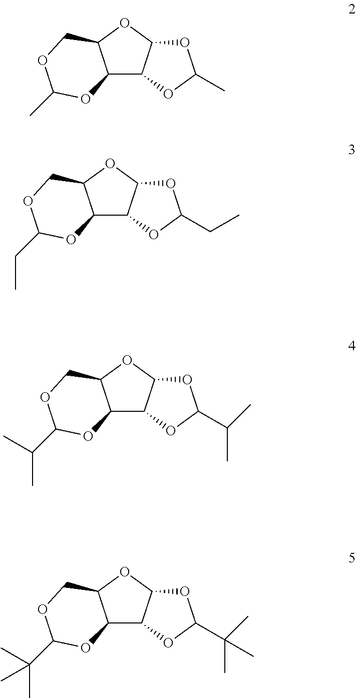

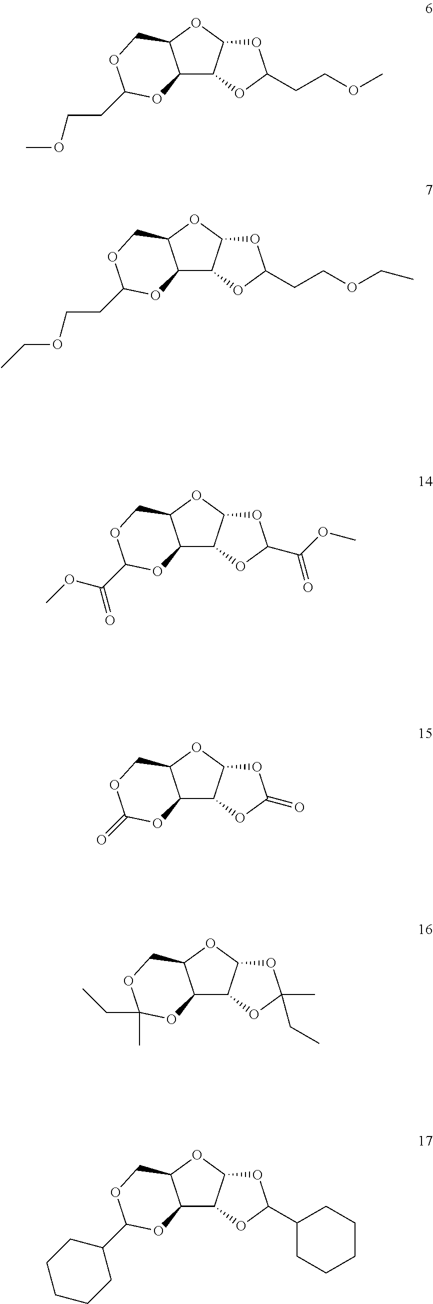

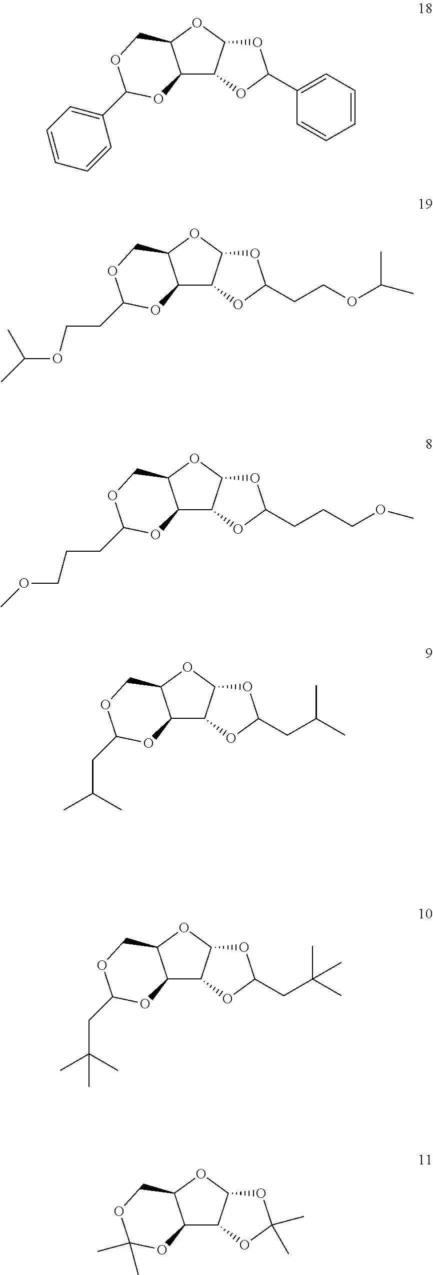

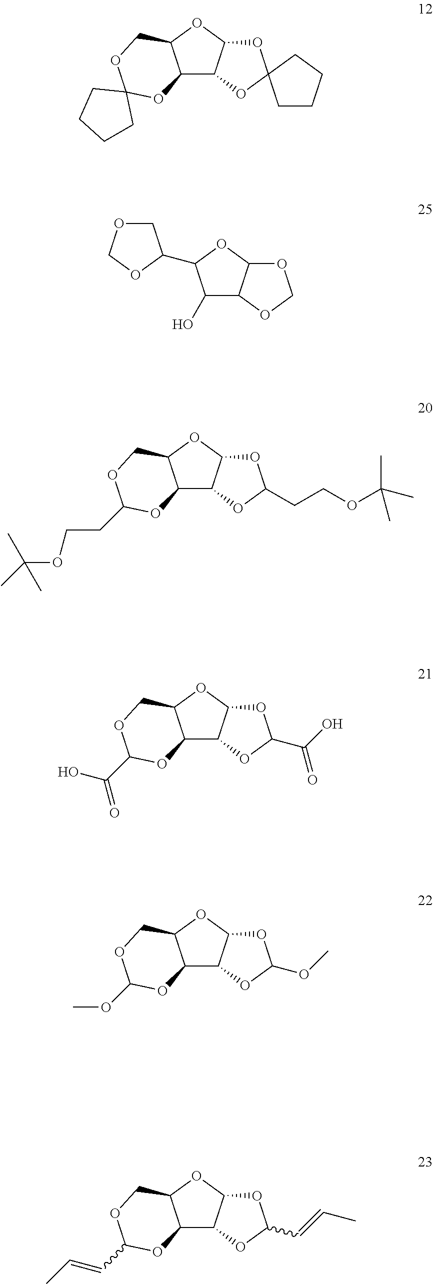

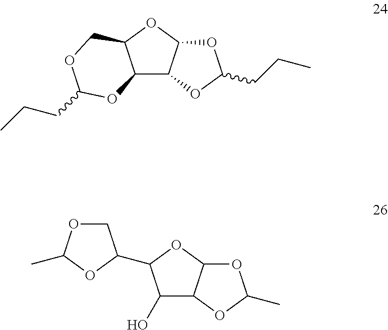

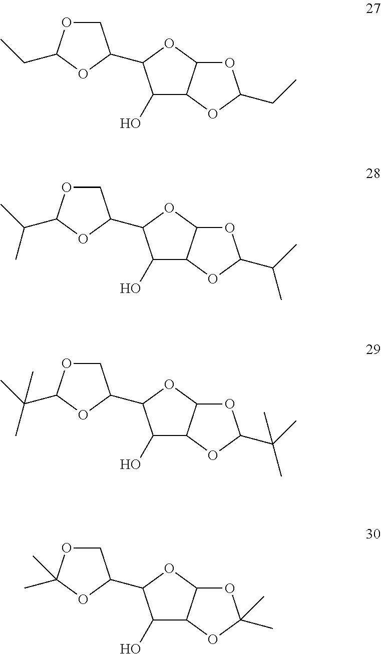

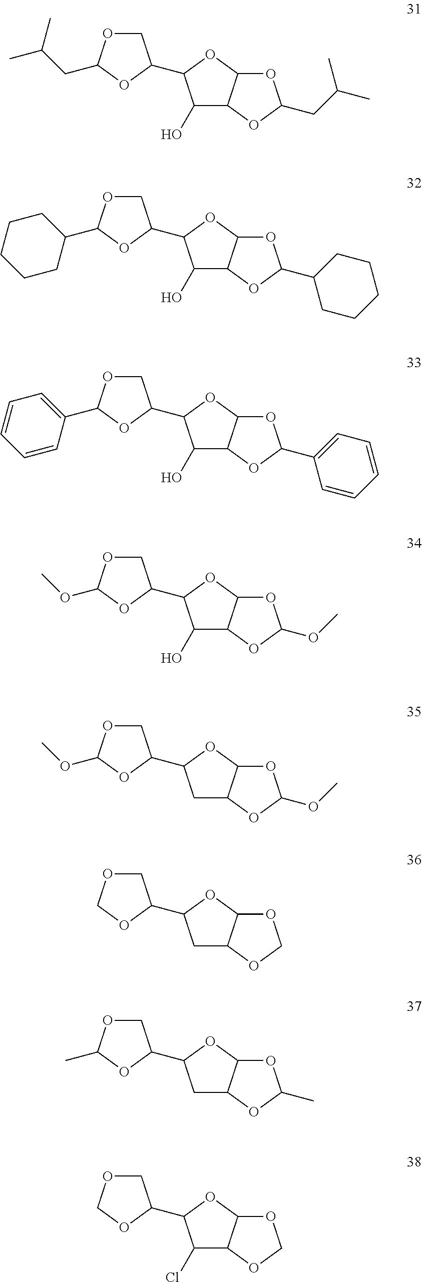

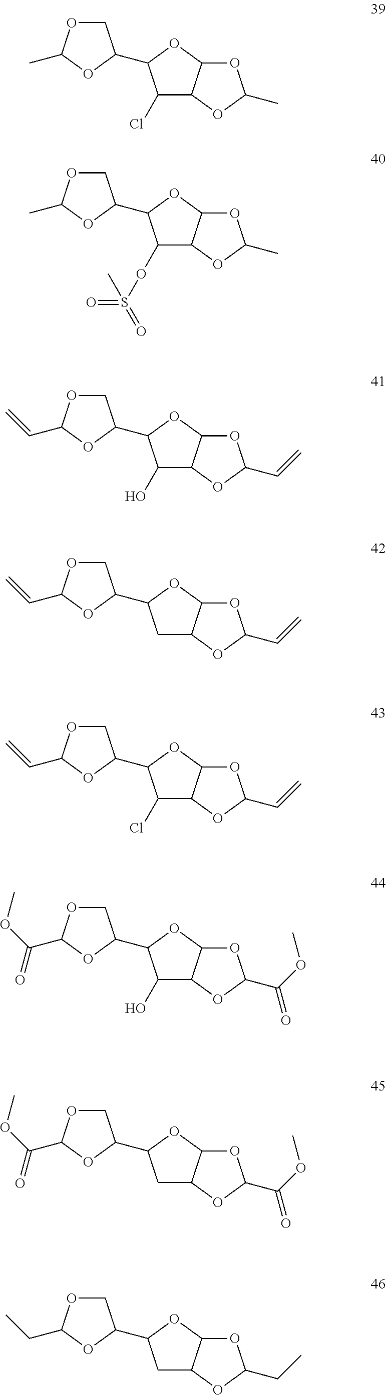

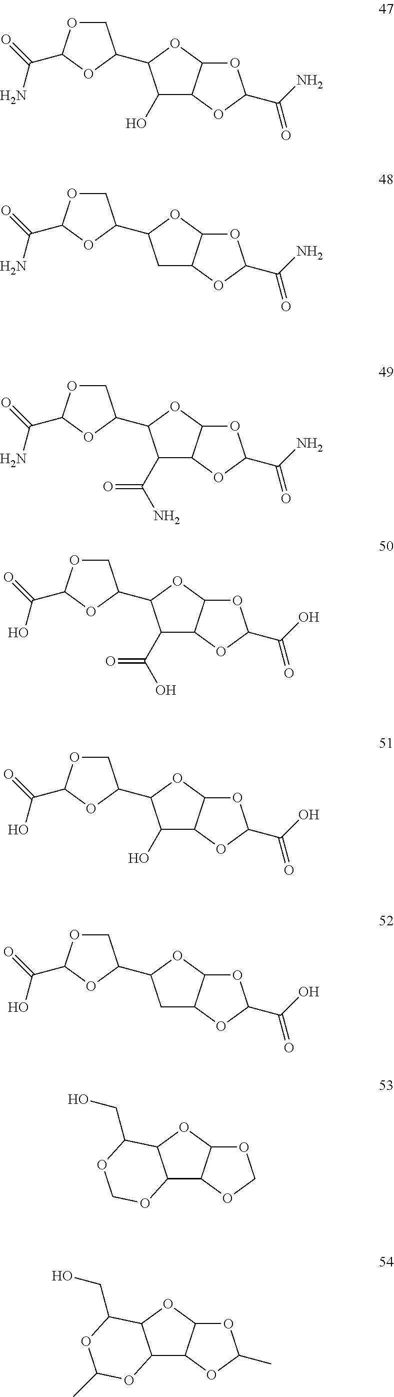

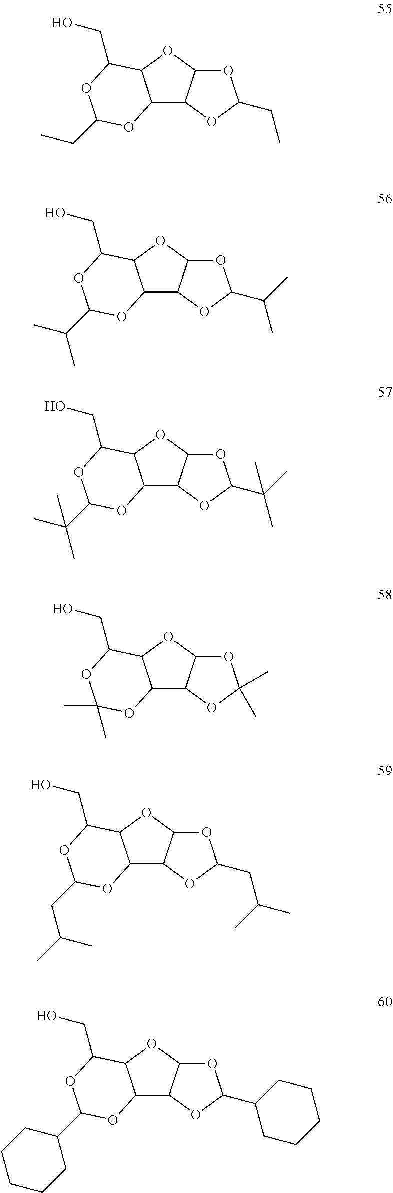

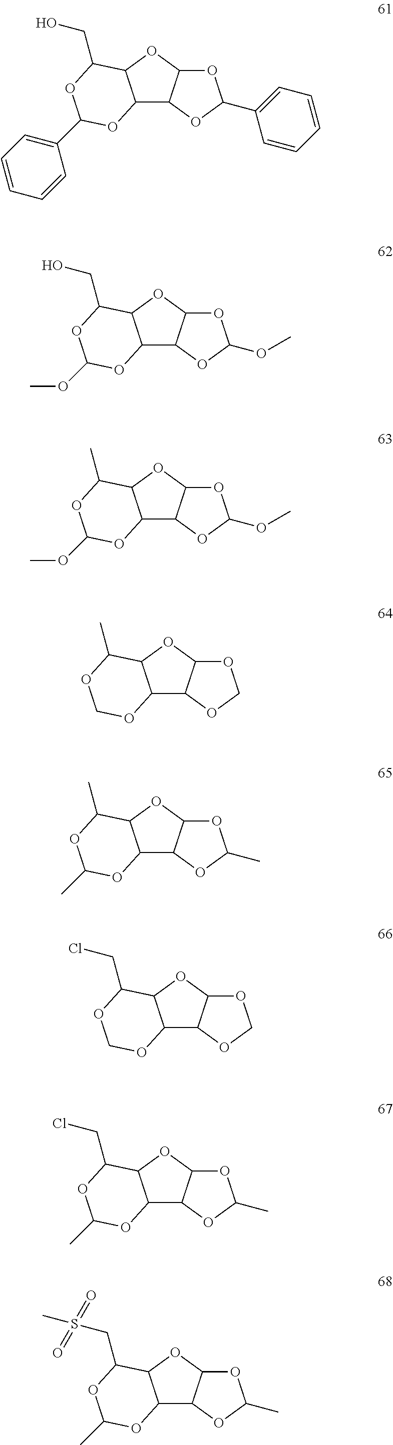

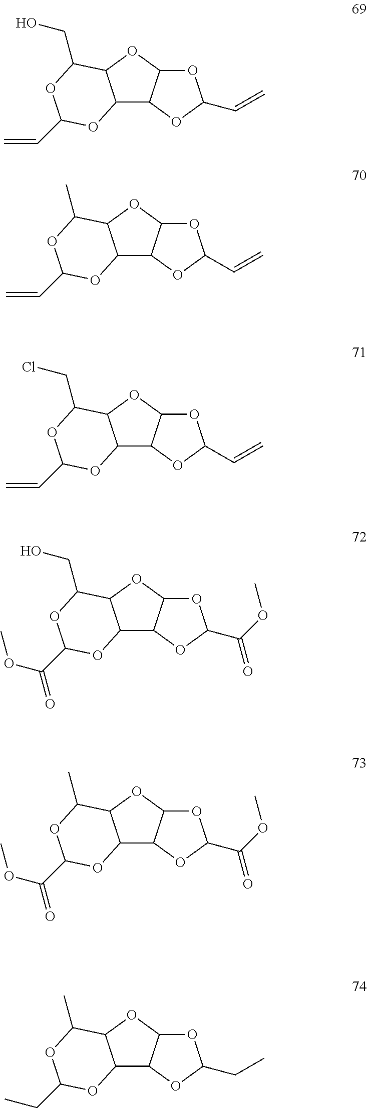

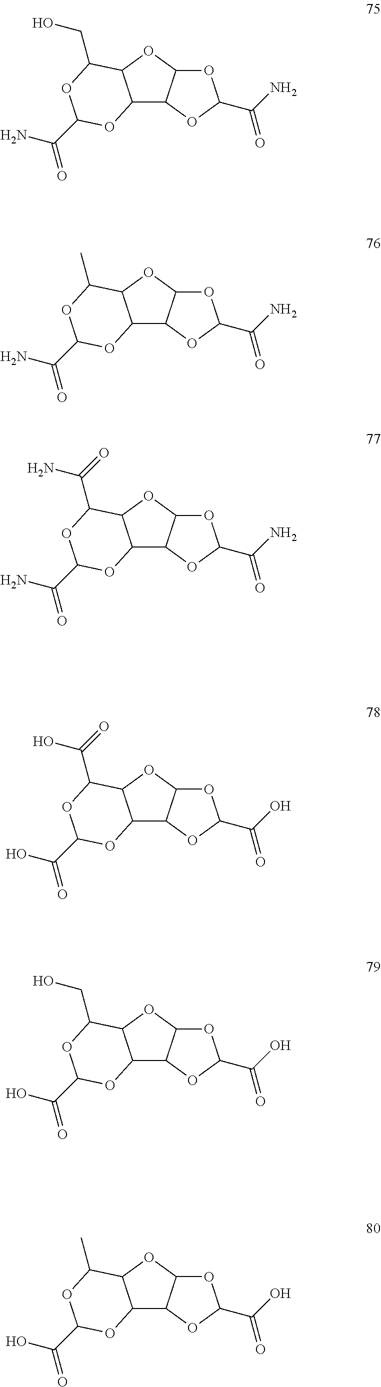 [SMILES: NC(=O)C1OC2OC3C(CO)OC(C(N)=O)OC3C2O1 ; CC1OC(C(N)=O)OC2C1OC1OC(C(N)=O)OC12 ; NC(=O)C1OC2OC3C(C(N)=O)OC(C(N)=O)OC3C2O1 ; O=C(O)C1OC2OC3C(C(=O)O)OC(C(=O)O)OC3C2O1 ; O=C(O)C1OC2OC3C(CO)OC(C(=O)O)OC3C2O1 ; CC1OC(C(=O)O)OC2C1OC1OC(C(=O)O)OC12]

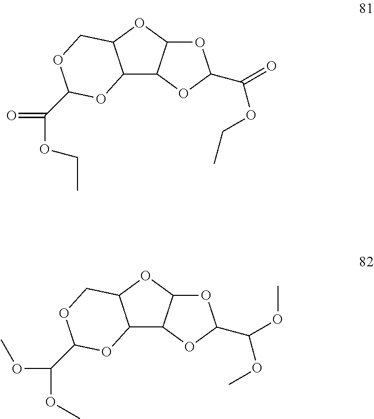

In an especially preferred embodiment, the solvent of the present invention is selected from the group consisting of diformyl xylose (DFX), dipropyl xylose, dibutyl xylose, diisobutyl xylose and dimethylglyoxylate xylose, preferably diformyl xylose. Due to its high melting point, diformyl xylose can replace conventional solvents that are solids at room temperature as well such as ethylene carbonate, N-formylmorpholine, cygnet and sulfolane.

For reactions having a reaction temperature below 50° C. however, compounds of the formula (I), wherein all of R₁, R₁′, R₂, and R₂′, are different from hydrogen are preferred.

Preferably, the compounds according to the present invention are used in large-scale processes, preferably in batch reactor tanks or continuous reactor tanks having a filling volume of at least 500 liters, preferably at least 1000 liters and most preferably 10000 liters. The compounds according to the present invention can potentially be manufactured in high quantities for a reasonably price utilizing different biomass sources. Therefore, production of compounds according to the present invention either from lignocellulosic feedstock or D-xylose would not only be sustainable, but also economically feasible.

Preferably, the polar aprotic solvents according to the present invention, most preferably the compounds of formula (I) or (II) are used a replacement of N-methylpyrrolidinone, N,N-dimethylacetamide (DMAc) or N,N-dimethylformamide (DMF) allowing to provide a green solvent substitute for said very toxic solvents.

Due to their excellent combination of solvent properties in terms of polarity, boiling point, melting point, and viscosity the polar, protic solvents of the present invention can be used for a broad variety of chemical reaction. Especially good results could be obtained for reactions selected from the group consisting of reduction and in particular the reduction of nitro compounds, hydrogenation, halogenation, synthesis of ionic liquids, nucleophilic substitution with SN1 mechanism, elimination reaction with E1 mechanism and E2, in particular if a very strong base is used, synthesis via SnAr mechanism, and extractions of bioactive compounds or any compounds from biological sources. The polar, protic solvents of the present invention are particularly preferred for extraction of bioactive compounds or any compounds from biological sources because they contain both polar and non polar groups which make them able to extract both polar and non polar compounds.

The term bioactive compounds as used throughout this application refers to compounds that interact with a living organism such as plants, bacteria, viruses, animals and humans. Exemplary bioactive compounds are carbohydrates, carboxylic acids, lignin and alcohols. The majority of bioactive compounds are well dissolved by protic solvents due to hydrogen bonding between solvent and solute.

The compounds according to the present invention can be produced during aldehyde-assisted lignocellulosic biomass processing, at a yield close to 95-99% of initially appearing in biomass xylan (M. Talebi Amiri, G. R. Dick, Y. M. Questell-Santiago and J. S. Luterbacher, Nat. Protoc., 2019, 14, 921-954.). They can also be directly synthesized from D-xylose with an aldehyde, a ketone or a carbonate in the presence of HCl using 1,4-dioxane as a solvent (Y. M. Questell-Santiago, R. Zambrano-Varela, M. Talebi Amiri and J. S. Luterbacher, Nat. Chem., 2018, 10, 1222-1228).

According to a further aspect of the invention, the compounds according to the present invention can be produced by reacting D-xylose or D-glucose with an aldehyde, a ketone or a carbonate in the presence of H₂SO₄ using 2-Me-THE as a solvent preferably at about 80° C. These modifications allow to use the green solvent 2-MeTHF in the reaction instead of 1,4-dioxane, a carcinogen linked to organ toxicity and environmental contaminant. Moreover, significantly less amount of solvent can now be used (3-times less by volume) to achieve the same yield (75-80%).

One embodiment of the present invention relates a method for producing a compound of the general formula (I), (II), (III) (VI), (V) or (VI)

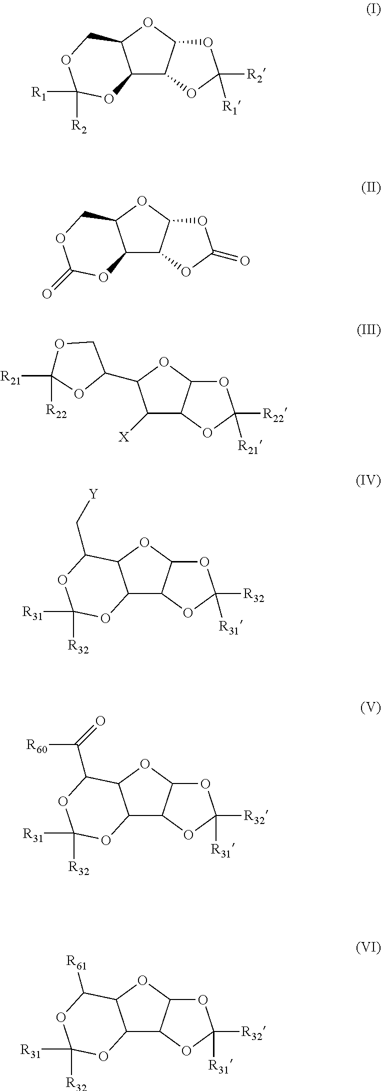

wherein R₁ and R₁′, R₂₁ and R₂₁′, R₃₁ and R₃₁′ are the same and are hydrogen, a linear or branched C₁ to C₁₈ alkyl, a linear or branched C₂ to C₁₈ alkenyl, a linear or branched C₁ to C₁₀ alkoxy, a linear or branched C₁ to C₉ alkanoyloxy, a linear or branched C₁ to C₉ alkoxycarbonyl, aminocarbonyl, hydroxycarbonyl, a linear or branched C₁ to C₉ alkoxyalkyl, an unsubstituted cycloalkyl, a linear or branched C₁ to C₆ alkyl substituted cycloalkyl, preferably an unsubstituted cyclohexyl, an unsubstituted C₆ to C₁₂ aryl, a linear or branched C₁ to C₄ alkyl substituted C₆ to C₁₂ aryl, or C₇ to C₁₂ aralkyl,

R₂ and R₂′, R₂₂ and R₂₂′, R₃₂ and R₃₂′ are the same and are hydrogen, a linear or branched C₁ to C₁₈ alkyl, a linear or branched C₁ to C₉ alkoxyalkyl, or R₂ and R₂′ form together with R₁ and R₁′ respectively, R₂₂ and R₂₂′ form together with R₂₁ and R₂₁′ respectively, R₃₂ and R₃₂′ form together with R₁ and R₁′ respectively, a 5 or 6 membered unsaturated or saturated carbocyclic ring optionally comprising 1 or 2 oxygen atoms,

X is selected from the group consisting of hydrogen, fluoro, chloro, bromo, iodo, a linear or branched C₁ to C₉ alkanoyloxy, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl,

Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl,

R₆₀ is selected from the group consisting of hydrogen, hydroxy and C₁ to C₁₈ alkoxy, and

R₆₁ is selected from the group consisting of hydrogen, hydroxy, C₁ to C₁₀ alkylsulfonyl-C₁ to C₅ (preferably methylsulfonylmethyl) and C₁ to C₁₈ alkyl
involving the reaction step of
reacting D-xylose or D-glucose with
an aldehyde of the general formula (XXX) R₁₁CHO, (XXXI) R₂₁CHO or (XXXII) R₃₁CHO, or
a ketone of the general formula (XXIII) R₁₁COR₁₂, (XXIV) R₂₁COR₂₂ or (XXV) R₃₁COR₃₂ or
a cyclic or linear carbonate
and R₁₁, R₁₂, R₂₁, R₂₂, R₃₁ and R₃₂ have the same definition as above,
in the presence of H₂SO₄.

The following reactions and their products are preferred:

| Starting | Reaction | Exemplary | |
|---|---|---|---|
| compound | with | reaction | Exemplary product |
| | Linear aldehyde | 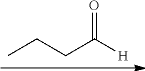 | 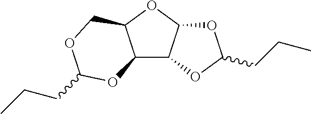 |
| 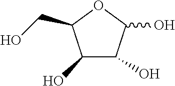 | Branched aldehyde | 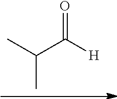 | 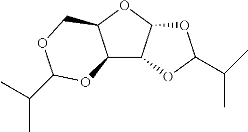 |
| 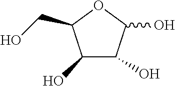 | Alkoxy-alkyl- aldehyde | 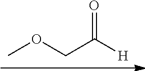 | 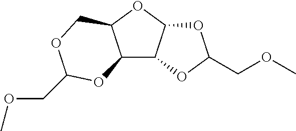 |
| 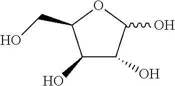 | Ketone | 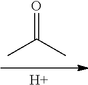 | 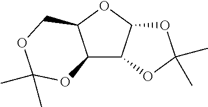 |
| 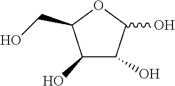 | Cyclic ketone | 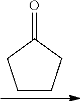 | 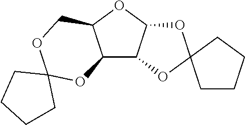 [SMILES: C1CCC2(C1)OC[C@H]1O[C@@H]3OC4(CCCC4)O[C@@H]3[C@H]1O2] |
| 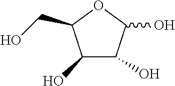 | Cyclic carbonate | 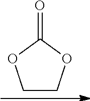 | 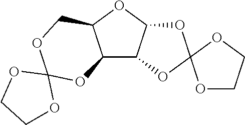 [SMILES: C1COC2(O1)OC[C@H]1O[C@@H]3OC4(OCCO4)O[C@@H]3[C@H]1O2] |
| 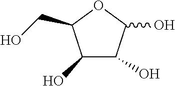 | Ketoester | 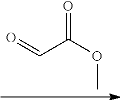 | 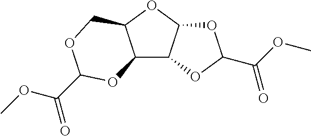 |
| 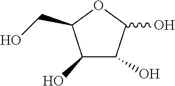 | Linear carbonate | 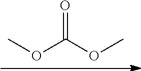 | 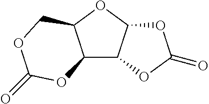 [SMILES: O=C1OC[C@H]2O[C@@H]3OC(=O)O[C@@H]3[C@H]2O1] |
| 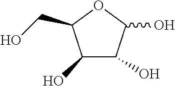 | Linear aldehyde with double bond | 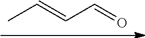 | 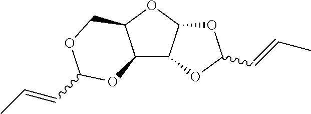 |
| 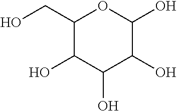 [SMILES: OCC1OC(O)C(O)C(O)C1O] | Linear aldehyde | 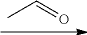 | 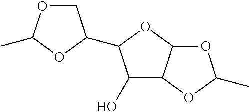 |
| 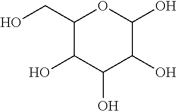 [SMILES: OCC1OC(O)C(O)C(O)C1O] | 1. Linear aldehyde 2. p-TsCl or MsCl, Et₃N 3. NaBH₄ | 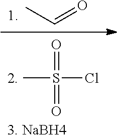 | 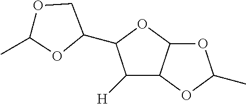 |
| 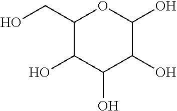 [SMILES: OCC1OC(O)C(O)C(O)C1O] | 1. Linear aldehyde 2. HCl | 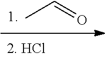 | 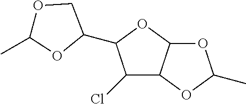 |
| 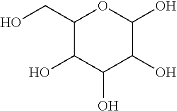 [SMILES: OCC1OC(O)C(O)C(O)C1O] | 1. Linear aldehyde 2. MsCl, Et₃N | 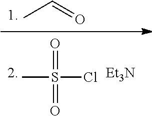 | 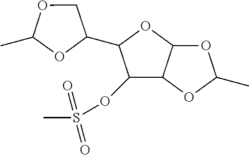 |
| 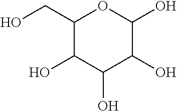 [SMILES: OCC1OC(O)C(O)C(O)C1O] | Linear aldehyde with double bond | 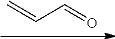 | 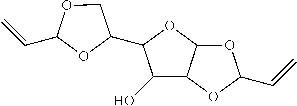 [SMILES: C=CC1OCC(C2OC3OC(C=C)OC3C2O)O1] |
| 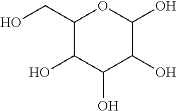 [SMILES: OCC1OC(O)C(O)C(O)C1O] | 1. Linear aldehyde with double bond 2. p-TsCl or MsCl, Et₃N 3. NaBH₄ | 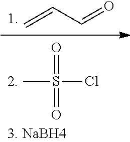 | 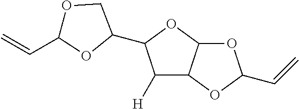 [SMILES: C=CC1OCC(C2CC3OC(C=C)OC3O2)O1] |
| 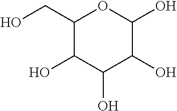 [SMILES: OCC1OC(O)C(O)C(O)C1O] | 1. Linear aldehyde with double bond 2. HCl | 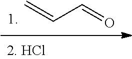 | 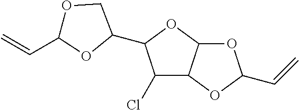 [SMILES: C=CC1OCC(C2OC3OC(C=C)OC3C2Cl)O1] |
| 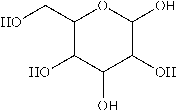 [SMILES: OCC1OC(O)C(O)C(O)C1O] | 1. Linear aldehyde with double bond 2. MsCl, Et₃N | 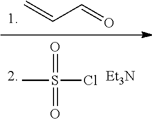 | 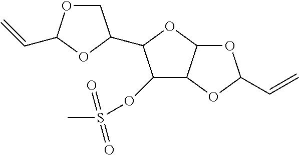 [SMILES: C=CC1OCC(C2OC3OC(C=C)OC3C2OS(C)(=O)=O)O1] |
| 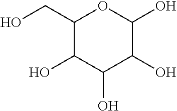 [SMILES: OCC1OC(O)C(O)C(O)C1O] | Alkoxy-alkyl- aldehyde | 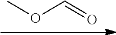 | 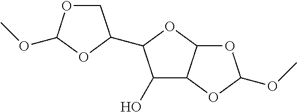 |
| 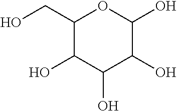 [SMILES: OCC1OC(O)C(O)C(O)C1O] | 1. Alkoxy-alkyl- aldehyde 2. p-TsCl or MsCl, Et₃N 3. NaBH₄ | 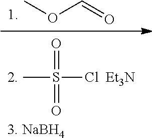 | 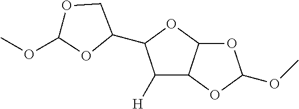 |
| 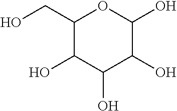 [SMILES: OCC1OC(O)C(O)C(O)C1O] | 1. Alkoxy-alkyl- aldehyde 2. HCl | 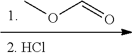 | 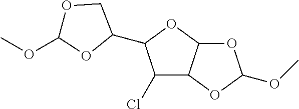 |
| 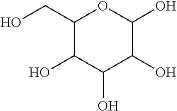 [SMILES: OCC1OC(O)C(O)C(O)C1O] | 1. Alkoxy-alkyl- aldehyde 2. MsCl, Et₃N | 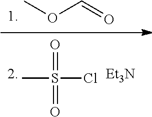 | 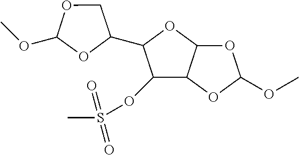 |
| 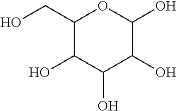 [SMILES: OCC1OC(O)C(O)C(O)C1O] | Ketoester | 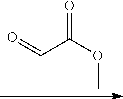 | 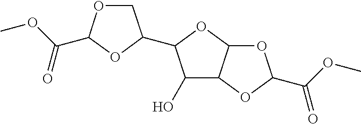 |
| 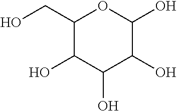 [SMILES: OCC1OC(O)C(O)C(O)C1O] | 1. Ketoester 2. 2. p-TsCl or MsCl, Et₃N 3. NaBH₄ | 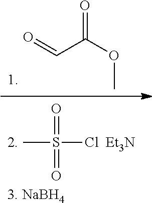 | 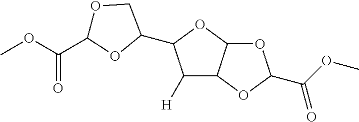 |
| 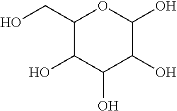 [SMILES: OCC1OC(O)C(O)C(O)C1O] | 1. Ketoester 2. 2. p-TsCl or MsCl, Et₃N 3. HCl | 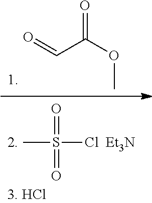 | 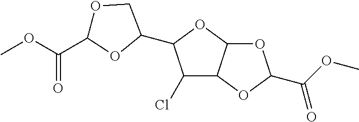 |
| 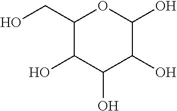 [SMILES: OCC1OC(O)C(O)C(O)C1O] | 1. Ketoester 2. MsCl, Et₃N | 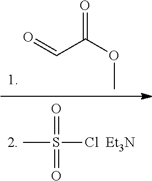 | 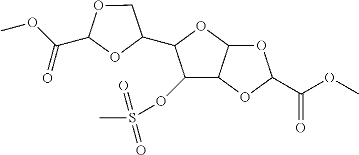 |
| 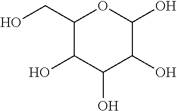 [SMILES: OCC1OC(O)C(O)C(O)C1O] | 1. Alkoxy-alkyl- aldehyde 2. p-TsCl or MsCl, Et₃N 3. NaBH₄ | 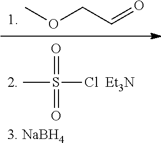 | 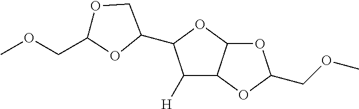 |
| 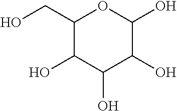 [SMILES: OCC1OC(O)C(O)C(O)C1O] | 1. Alkoxy-alkyl- aldehyde 2. MsCl, Et₃N 3. HCl | 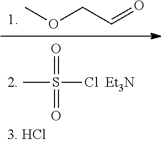 | 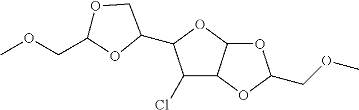 |
| 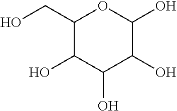 [SMILES: OCC1OC(O)C(O)C(O)C1O] | 1. Alkoxy-alkyl- aldehyde 2. MsCl, Et₃N | 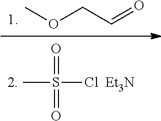 | 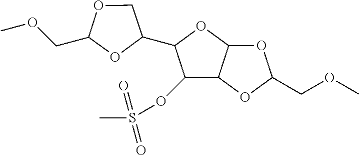 |
| 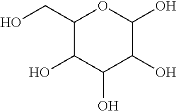 [SMILES: OCC1OC(O)C(O)C(O)C1O] | 1. Linear aldehyde 2. p-TsCl or MsCl, Et₃N 3. NaBH₄ | 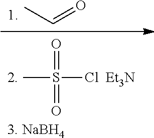 | 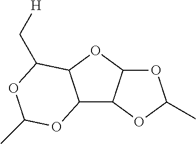 |
| 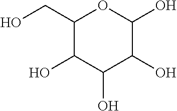 [SMILES: OCC1OC(O)C(O)C(O)C1O] | 1. Linear aldehyde 2. HCl | 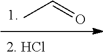 | 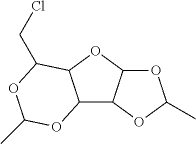 |
| 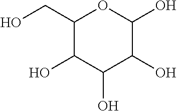 [SMILES: OCC1OC(O)C(O)C(O)C1O] | 1. Linear aldehyde 2. MsCl, Et₃N | 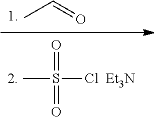 | 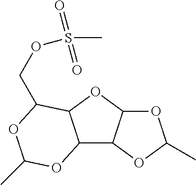 |
| 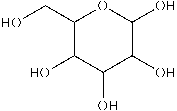 [SMILES: OCC1OC(O)C(O)C(O)C1O] | 1. Linear aldehyde with double bond 2. p-TsCl or MsCl, Et₃N 3. NaBH₄ | 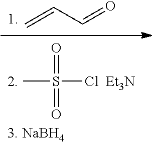 | 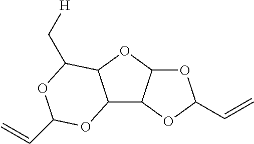 [SMILES: C=CC1OC2OC3C(C)OC(C=C)OC3C2O1] |
| 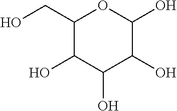 [SMILES: OCC1OC(O)C(O)C(O)C1O] | 1. Linear aldehyde with double bond 2. HCl | 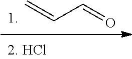 | 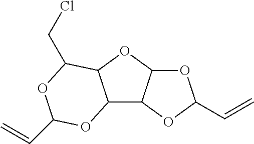 [SMILES: C=CC1OC2OC3C(CCl)OC(C=C)OC3C2O1] |
| 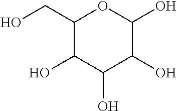 [SMILES: OCC1OC(O)C(O)C(O)C1O] | 1. Linear aldehyde with double bond 2. MsCl, Et3N | 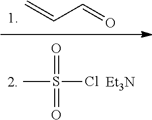 | 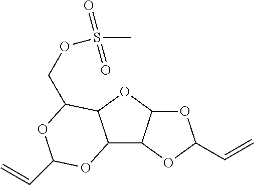 [SMILES: C=CC1OC2OC3C(COS(C)(=O)=O)OC(C=C)OC3C2O1] |
| 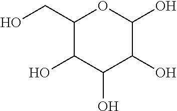 [SMILES: OCC1OC(O)C(O)C(O)C1O] | 1. Linear aldehyde with double bond 2. p-TsCl or MsCl, Et₃N 3. NaBH₄ | 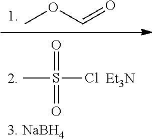 | 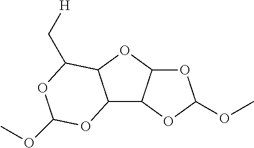 |
| 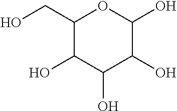 [SMILES: OCC1OC(O)C(O)C(O)C1O] | 1. Linear aldehyde with double bond 2. HCl | 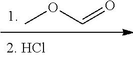 | 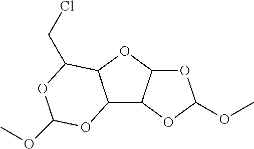 |
| 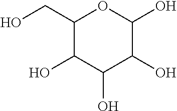 [SMILES: OCC1OC(O)C(O)C(O)C1O] | 1. Linear aldehyde with double bond 2. MsCl, Et₃N | 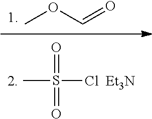 | 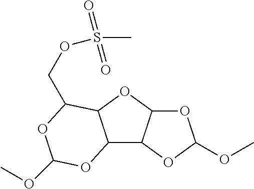 |
| 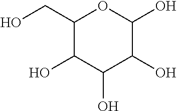 [SMILES: OCC1OC(O)C(O)C(O)C1O] | 1. Linear aldehyde with double bond 2. p-TsCl or MsCl, Et₃N 3. NaBH₄ | 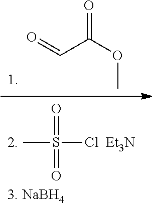 | 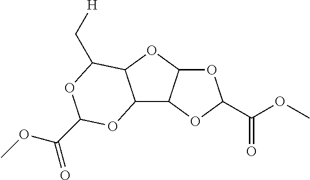 |
| 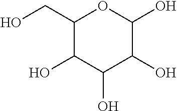 [SMILES: OCC1OC(O)C(O)C(O)C1O] | 1. Linear aldehyde with double bond 2. HCl | 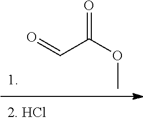 | 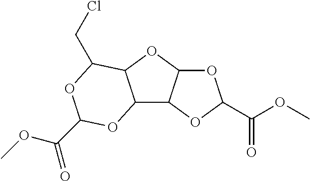 |
| 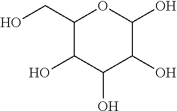 [SMILES: OCC1OC(O)C(O)C(O)C1O] | 1. Linear aldehyde with double bond 2. MsCl, Et₃N | 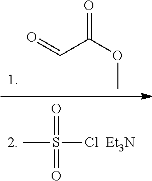 | 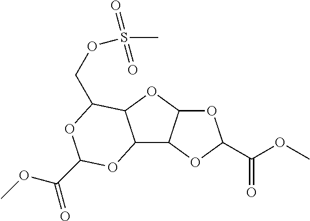 |
| 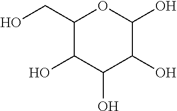 [SMILES: OCC1OC(O)C(O)C(O)C1O] | 1. Linear aldehyde with double bond 2. p-TsCl or MsCl, Et₃N 3. NaBH₄ | 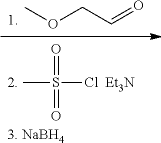 | 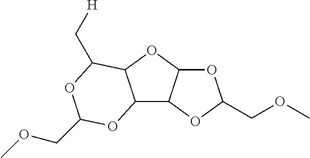 |
| 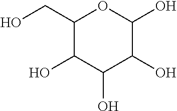 [SMILES: OCC1OC(O)C(O)C(O)C1O] | 1. Linear aldehyde with double bond 2. HCl | 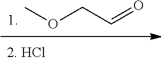 | 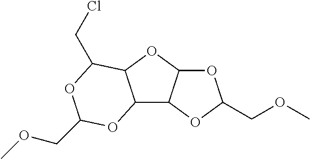 |
| 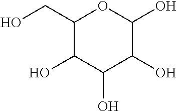 [SMILES: OCC1OC(O)C(O)C(O)C1O] | 1. Linear aldehyde with double bond 2. MsCl, Et₃N | 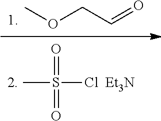 | 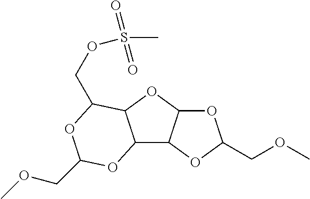 |

The present invention also relates to the following new compounds of formula (I):

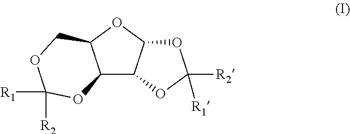

wherein R₁ and R₁′ are the same and are a linear or branched C₁ to C₁₀ alkyl, R₂ and R₂′ are the same and are a linear or branched C₁ to C₁₀ alkyl or

R₂ and R₂′ form together with R₁ and R₁′ respectively, a 5 or 6 membered unsaturated carbocyclic ring or a 5 or 6 membered saturated carbocyclic ring optionally comprising 1 or 2 oxygen atoms, as for example shown in compound 13.

Especially preferred are compounds of formula I wherein R₂ and R₂′ form together with R₁ and R₁′ respectively, a 5 or 6 membered unsaturated or saturated carbocyclic ring optionally comprising 1 or 2 oxygen atoms. Examples of this type of compound are compound 12 and compound 13.

The method of the present invention involves the reaction step of
reacting D-xylose or D-glucose with
an aldehyde of the general formula (XXX) R₁₁CHO, (XXXI) R₂₁CHO or (XXXII) R₃₁CHO, or
a ketone of the general formula (XXIII) R₁₁COR₁₂, (XXIV) R₂₁COR₂₂ or (XXV) R₃₁COR₃₂ or
a cyclic or linear carbonate
and R₁₁, R₁₂, R₂₁, R₂₂, R₃₁ and R₃₂ have the same definition as above,
in the presence of H₂SO₄ using 2-methyltetrahydrofuran (2-Me-THF) as a solvent.

This allows direct production of the compounds defined in claim 16 from D-xylose or D-glucose, in particular direct production of DFX form D-xylose using safer, more environmental friendly chemicals compared to the prior art (solid paraformaldehyde powder instead of aqueous formaldehyde solution, sulfuric acid instead of aqueous solution of HCl, 2-MeTHE instead of 1,4-dioxane).

According to a preferred embodiment of the method of the present invention, the aldehyde is paraformaldehyde.

Using concentrated (98-99 wt %) H₂SO₄ instead of 37 wt % aqueous solution of HCl and paraformaldehyde instead of formaldehyde (37 wt % aqueous solution formalin) allows to eliminate water from the synthesis and to use green solvent 2-Me-THE, which is not miscible with water, in the synthesis instead of 1,4 dioxane, a carcinogen linked to organ toxicity and an environmental contaminant. Moreover, significantly less amount of solvent can now be used (3-times less by volume) to achieve the same yield (75-80%). The new method enables much easier and less time- and labour-consuming workup at the end of the synthesis by excluding distillation and extraction steps as the residue is rapidly crystallizing directly from the final oil.

The method advantageously comprises the steps of
a) providing a composition containing D-xylose or D-glucose, an aldehyde and 2-methyltetrahydrofuran (2-Me-THE),
b) adding H₂SO₄ to the composition of step a)
c) heating the composition of step b).

According to an embodiment of the present invention the method may not comprise an extraction or distillation step. This embodiment is specifically preferred for the production of DFX.

According to an alternative embodiment the method may further comprise the steps of
d) extracting the compound a compound of the general formula (I), (II), (III) (VI), (V) or (VI) using at least one extraction solvent.
e) distilling the product from the extracted organic residue.

According to a preferred embodiment of the present invention, ethyl acetate and/or cyclopentyl methyl ether (CPME) are used as extraction solvents. These extraction solvents are safer than n-hexane, a known neurotoxin which is used in prior art as extraction solvent.

### FIGURES

FIGS. 1A to 1D show the optimization of reaction conditions for direct synthesis of diformylxylose from D-xylose;

FIG. 2 shows a model alkylation (Menshutkin) reaction with the relationship between solvent polarity and the natural logarithm of the rate of reaction;

FIG. 3 shows rate constants for Menshutkin reaction in DFX, DPX, and DMGX at 90° C.;

FIG. 4A shows the conversion of cinnamaldehyde after hydrogenation in DFX and conventional solvents;

FIG. 4B shows conversion of cinnamaldehyde in DFX as a function of time at different Pd/C catalyst loadings;

FIG. 5 shows a model Heck reaction with the relationship between solvent polarity and the natural logarithm of the rate of reaction;

FIG. 6 shows rate constants for Heck reaction in DFX, DPX, DMGX;

FIG. 7A to 7D shows the results of Ames test on DFX;

FIG. 8 shows hydrogenolysis yields of aromatic monomers for the extracted formaldehyde-stabilized lignins;

FIG. 9a shows the stability of pure diformylxylose under acidic conditions;

FIG. 9b shows the degradation pathway of DFX and its reversible trapping with formaldehyde;

FIG. 10a shows absorbance spectra of 4-nitroaniline dye in solvents 3, 4 and 24 of claim 13 as well as in DMSO;

FIG. 10b shows absorbance spectra of N,N-diethyl-4-nitroaniline dye in solvents 3, 4 and 24 of claim 13 as well as in DMSO;

FIG. 10c shows absorbance spectra of Nile Red dye in solvents 3, 4, 42 and DMSO;

FIG. 11 shows the plots of 1/concentration of 1-bromodecane as a function of time in cyclopentyl methyl ether (CPME);

FIG. 12 shows the plots of 1/concentration of 1-bromodecane as a function of time in DES composed of DFX and propyl guaiacol (DFX:PG);

FIG. 13 shows the plots of 1/concentration of 1-bromodecane as a function of time in DES composed of DFX and Phenol (DFX:PhOH);

FIG. 14 shows a model alkylation (Menshutkin) reaction with the relationship between solvent polarity and the natural logarithm of the rate of reaction in DPX, DBX, DIBX and in conventional solvents;

FIG. 15A shows mass spectra depicting fragmentation of molecule and molecular ion for Dipropylxylose;

FIG. 15B shows mass spectra depicting fragmentation of molecule and molecular ion for Dibutylxylose;

FIG. 15C shows mass spectra depicting fragmentation of molecule and molecular ion for Diisobutylxylose;

FIG. 15D shows mass spectra depicting fragmentation of molecule and molecular ion for Dineopentylxylose;

FIG. 16A shows proton nuclear magnetic resonance (¹H-NMR) spectra of synthesized Diformylxylose in CDCl₃.

FIG. 16B shows carbon-13 nuclear magnetic resonance (¹³C-NMR) spectra of synthesized Diformylxylose in CDCl₃.

FIG. 17. shows solvent map based on Kamlet-Abboud-Taft parameters β and π* comparing DEX, DPX, DBX, DIBX to other solvents.

### EXAMPLES

### Example 1: Preparation of Diformyl Xylose (DFX)

D-xylose (15 g, 0.1 mol, 1.0 equiv.) and paraformaldehyde (7.5 g, equivalent to 0.25 mol formaldehyde, 2.5 equiv.) were added to 2-Me-THF (75 mL) in a round bottom flask. Then, H2SO4 (98 wt %, 2.46 mL, 0.045 mol, 0.45 equiv.) was added drop-wise with stirring to avoid the localized concentration of acid, which can degrade the sugar. The mixture was then heated to 80° C. for 3 h with stirring. The resulting solution was cooled to room temperature (˜23-25° C.), neutralized with sodium hydroxide saturated aqueous solution, filtered, and concentrated in vacuo using a rotary evaporator with a bath temperature of 45° C. The residue was crystallized directly and washed with ethanol while filtering to remove impurities and by-products. The resulting DFX product is white crystalline solid (298% pure by 1H-NMR and GC-FID).

Alternatively, if the residue is not crystallizing from the final oil, the following workup can be done. Extract the residue three times with 100 ml of ethyl acetate (or 50 ml of cyclopentyl methyl ether) and 25 ml of water in a separatory funnel. The resulting solution can be distilled at 80° C., under reduced pressure (0.02 mbar) to obtain a light yellow solid. The solid can then be recrystallized in ethanol and dried in a vacuum desiccator, yielding the DFX as a white crystalline solid (298% pure by 1H-NMR and GC-FID).

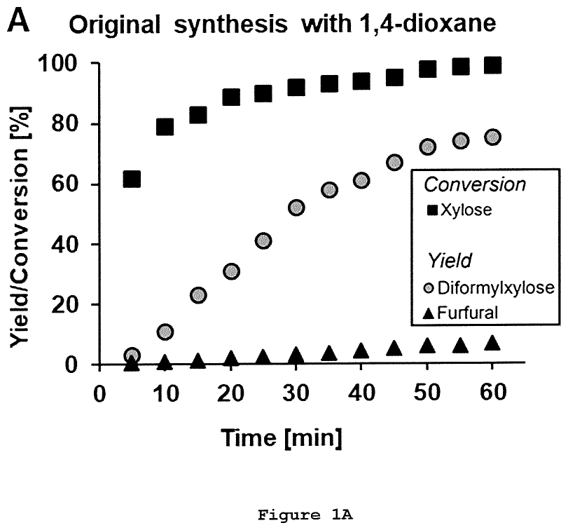

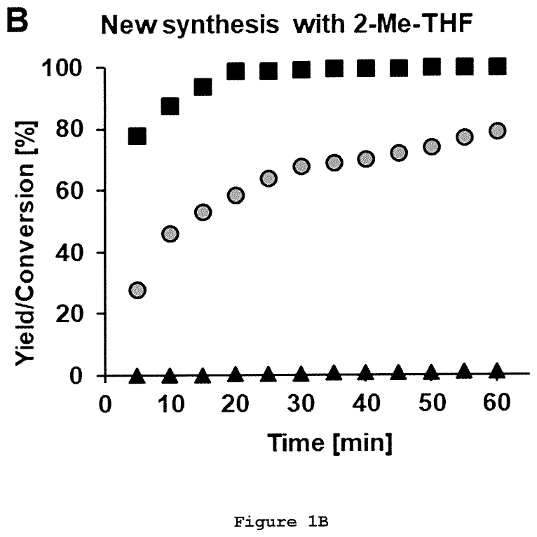

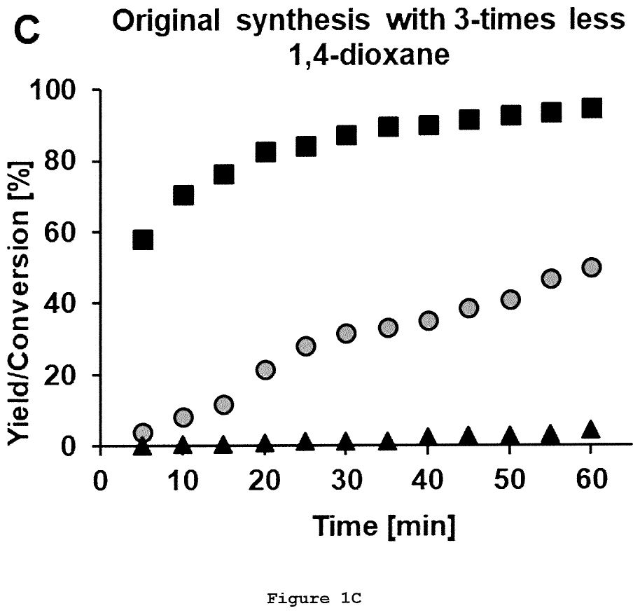

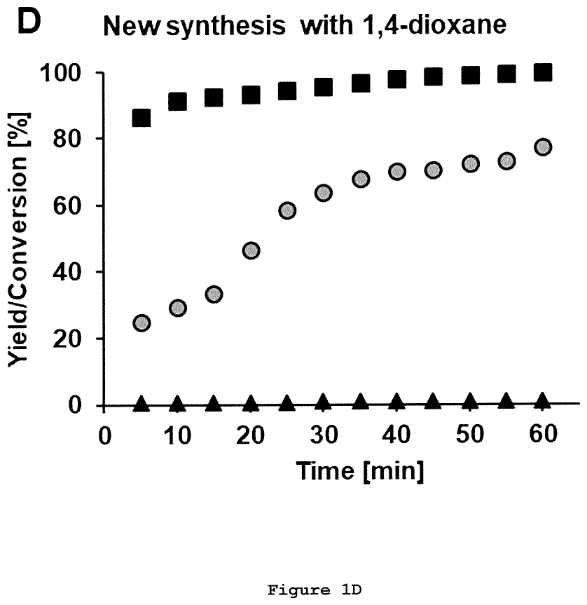

FIGS. 1A to 1D show the optimization of reaction conditions for direct synthesis of diformylxylose in 1,4-dioxane (A, C, D) or 2-Me-THF (B) at 80° C. using the conventional procedure (A, C) and the synthesis according to example 1 (B, D).

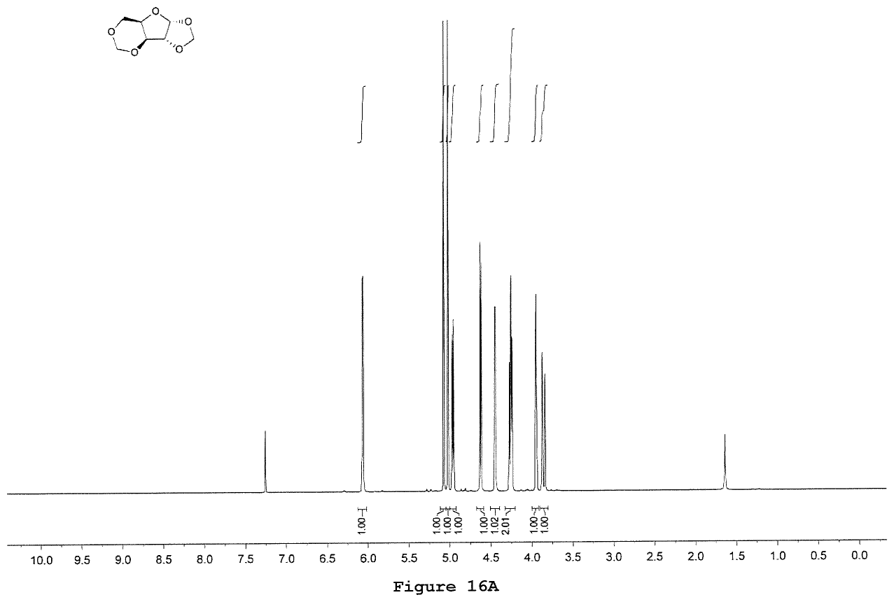

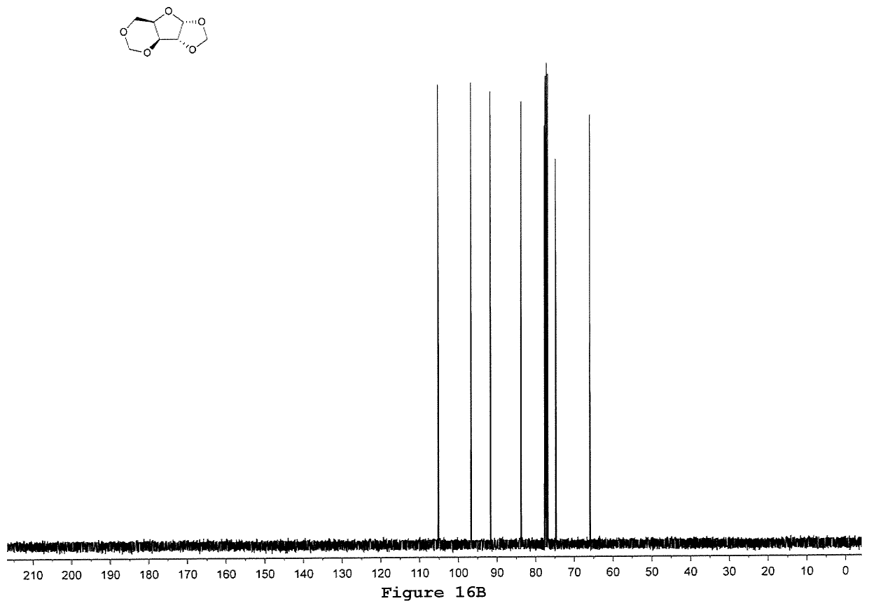

NMR spectra for the synthesized DFX are depicted in FIG. 16 A (proton NMR) and FIG. 16 B (carbon NMR).

The new synthesis to of DFX allows to use the green solvent 2-MeTHE instead of 1,4-dioxane. Moreover, significantly less amount of solvent can now be used (3-times less by volume) to achieve the same yield (75-80%). The new method enables much easier and less time- and labour-consuming workup at the end of the synthesis by excluding distillation and extraction steps. In alternative procedure, ethyl acetate or CPME can be used as extraction solvents instead of n-hexane (a known neurotoxin). Optimization studies reveal that the overall yield of DFX around 75-80% can be achieved after 50 min of the reaction. Also, the yield of furfural is almost negligible in the synthesis according to the present invention, meaning much less degradation of xylose occurred during the reaction.

### Example 2a: Preparation of Compounds of Formula (I), (III) and (IV)

D-xylose (35 g, 1.0 equiv.) and corresponding aldehyde, ketone, or diketoester (3.0 equiv.) were added to 1,4-dioxane (550 mL) in a 1 L round bottom flask. Then, HCl (37 wt %, 1.3 equiv.) was added. The mixture was then heated to 60° C. for 60 min with stirring. The resulting solution was cooled to room temperature (˜23-25° C.), neutralized with potassium bicarbonate, filtered, and concentrated in vacuo using a rotary evaporator with a bath temperature of 45° C. The residue was extracted three times with 100 ml of ethyl acetate (or 50 ml of cyclopentyl methyl ether) and 100 ml of water in a separatory funnel. The resulting solution was distilled at 80-95° C., under reduced pressure (0.02 mbar) to obtain a light yellow solid. The yield in all cases was >75%. The solid was then recrystallized in ethanol and dried in a vacuum desiccator, yielding the product as a white crystalline solid (298% pure by 1H-NMR and GC-FID).

Corresponding aldehydes are for example: benzaldehyde; cyclohexanal; acetaldehyde; propionaldehyde; butyraldehyde; valeraldehyde, isobutyraldehyde, pivaldehyde, 2-methoxyethanal, 2-ethoxyethanal, 2-propoxyethanal, 2-butoxyethanal, 2-pentoxyethanal, 2-hexoxyethanal, 2-heptoxyethanal, 2-phenoxyethanal and 2-benzyloxyethanal.

Corresponding ketones are for example: acetone, benzophenone, acetophenone, methyl ethyl ketone, methyl isobutyl ketone, methyl-sec-butylketone, cyclopentanone and cyclohexanone.

Corresponding diketoesters are α-diketoesters such as methyl pyruvate or ethylpyruvate or β-diketoesters such as ethyl acetoacetate or methyl acetoacetate.

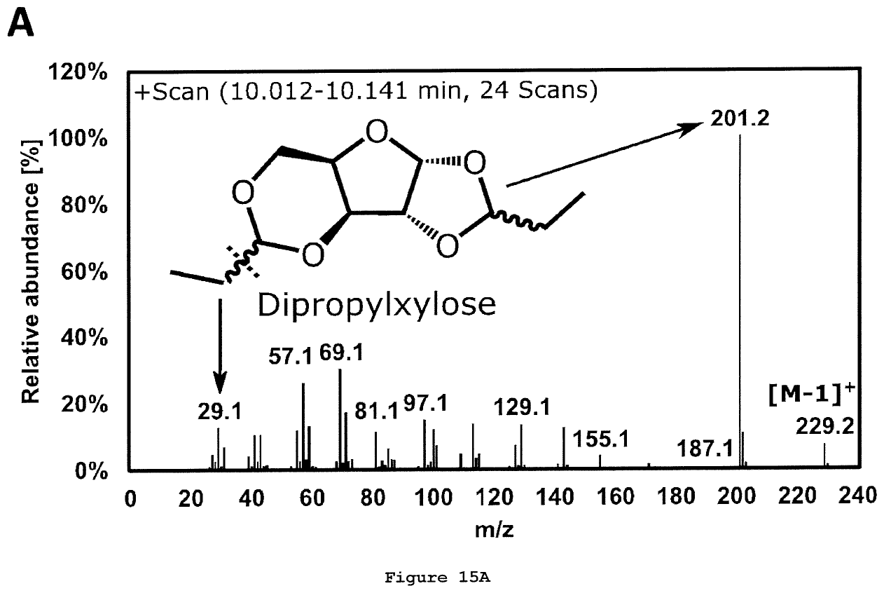

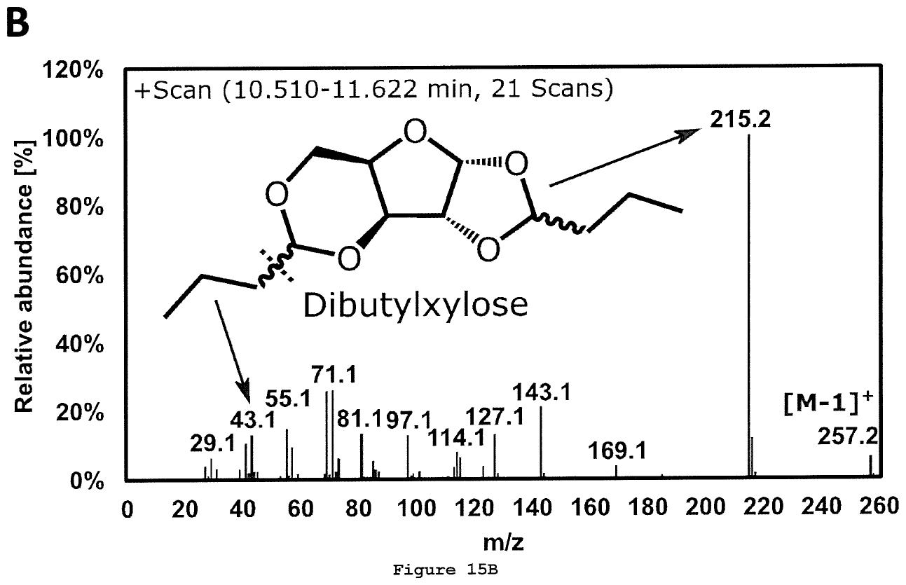

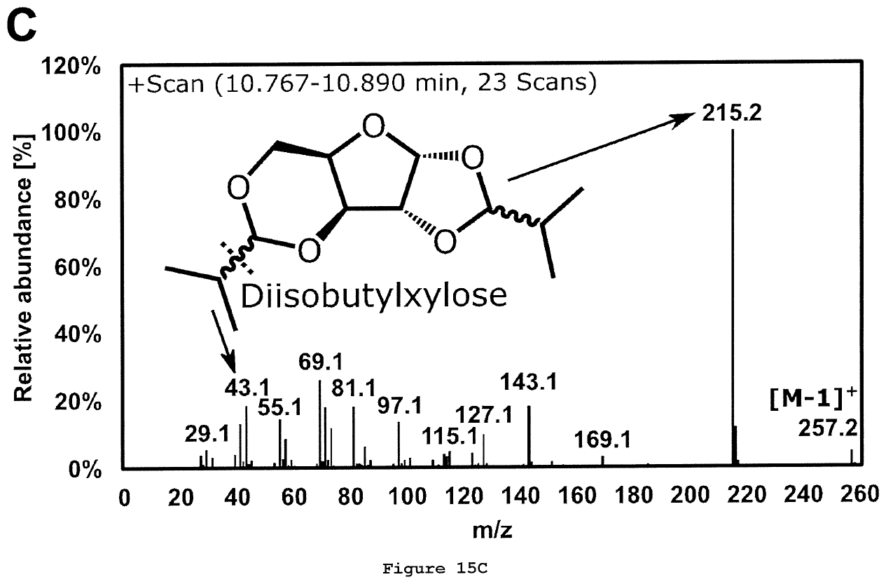

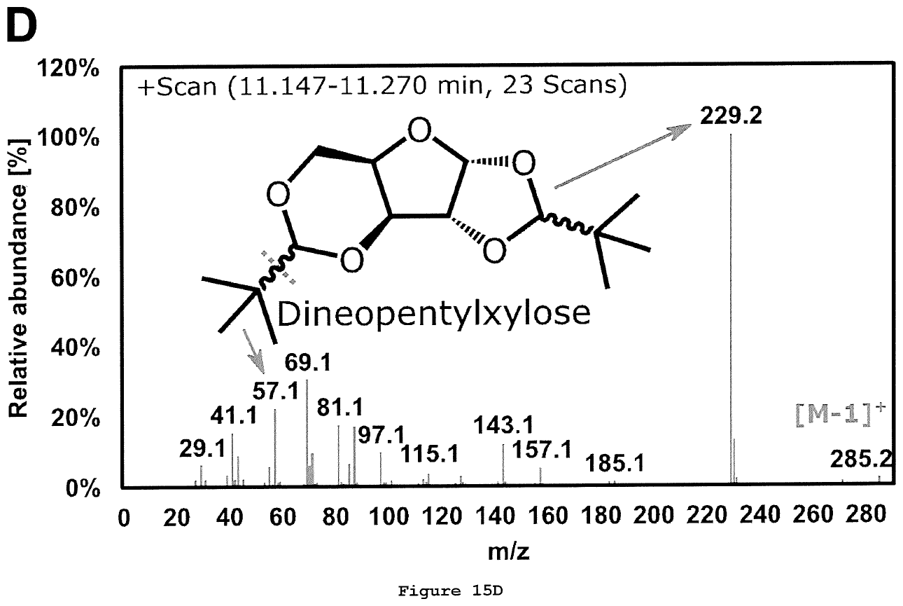

GC-MS spectra showing fragmentation of molecules and molecular ion of some of the synthesized compounds are depicted in FIGS. 15A, 15B, 15C and 15D. Specifically, in FIG. 15A for Dipropylxylose (DPX, compound I wherein each of R1 and R1′ is ethyl and each of R2 and R2′ is hydrogen), in FIG. 15B for Dibutylxylose (DBX, compound I wherein each of R1 and R1′ is propyl and each of R2 and R2′ is hydrogen), in FIG. 15C for Diisobutylxylose (DIBX, compound I wherein each of R1 and R1′ is isopropyl and each of R2 and R2′ is hydrogen) and in FIG. 15D for Dineopentylxylose (DNPX, compound I wherein each of R₁ and R₁′ is tert-butyl and each of R₂ and R₂′ is hydrogen).

### Example 2b: Preparation of Compounds of Formula II

1,2-O-methylene-α-D-xylofuranose (35 g, 1.0 equiv.) and corresponding carbonate (3.0 equiv.) were added to DMF (550 mL) in a 1 L round bottom flask. Then, NaOH (12 g, 1.3 equiv.) and catalyst TBD (1 mol %) was added. The mixture was then stirred for 4 h at 60° C. The resulting solution was quenched with acetic acid, filtered, and concentrated in vacuo using a rotary evaporator with a bath temperature of 45° C. The obtained mixture was directly purified by flash column chromatography on silica gel (hexane/ethyl acetate 1:6) to produce the product—O3,O5-carbonyl-O1,O2-methylene-α-D-xylofuranose.

The product (1 mol eq.) was then oxidized with KMnO4 (3 mol eq.) in the presence of phosphoric acid (2 mol eq.) and water. The reaction mixture was heated to 70° C. for 15 min to ensure solubilization of all components and then cooled down to 0° C. and stirred for 3 h. The mixtures was extracted 3 times with ethyl acetate and the organic phase was concentrated under vacuum to obtain the product in oil which then can be separated by flash chromatography or distilled.

Alternatively, D-xylose (35 g, 1.0 equiv.) and corresponding carbonate (3.0 equiv.) were added to DMF (550 mL) in a 1 L round bottom flask. Then, NaOH (12 g, 1.3 equiv.) and catalyst TBD (1 mol %) was added. The mixture was then stirred for 4 h at room temperature. The resulting solution was quenched with acetic acid, filtered, and concentrated in vacuo using a rotary evaporator with a bath temperature of 45° C. The obtained mixture was directly purified by flash column chromatography on silica gel (hexane/ethyl acetate 1:6)

Corresponding carbonates are for example 1, 3-dioxolan-2-on, diphenyl carbonate, dimethyl carbonate. Additionally, the following catalysts can be used: TBD, DMAP, Zn(OAc)₂, NaOCH₃, CS₂CO₃, K₂CO₃.

### Example 2c: Preparation of Diformylglucose Isomers

For preparation of compounds of formula (III) and (IV), in particular of diformylglucose isomers, specifically compounds 25 and 53, pure glucose (35 g) was reacted with 73 ml of 37 wt % HCl and 173 ml of 37 wt % FA in 1550 ml of 1,4-dioxane at 80° C. for 30 min. Before separation, the solution was neutralized with sodium bicarbonate and dried under reduced pressure with a rotary evaporator at 60° C. The residue was extracted five times with 500 ml of ethyl acetate. All organic phases were combined and dried under reduced pressure with a rotary evaporator set a 40° C. The resultant residue was distilled at 125° C. and ˜0.06 mbar to obtain a yellowish paste. To obtain pure diformylglucose isomers, the solution was separated by HPLC and targeted peaks were collected using an automated fraction collector.

### Example 3: Alkylation (Menshutkin) Reaction

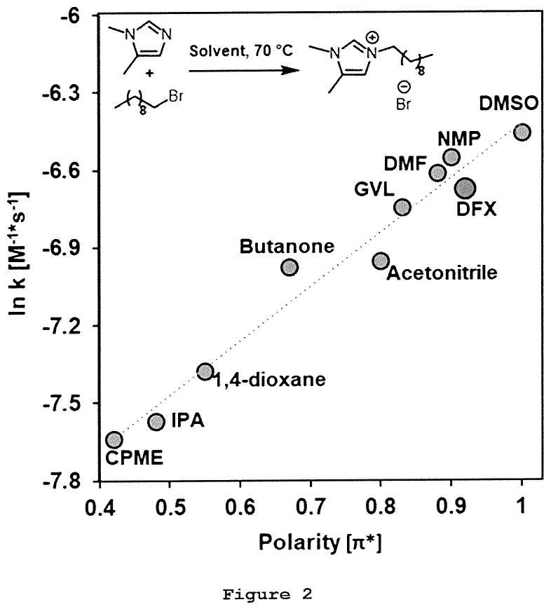

The reaction was performed between 1,2-dimethylimidazole (0.320 g, 3.33 mmol) and 1-bromodecane (0.65 ml, 3.13 mmol) in the chosen solvent (3 ml) with stirring at 70° C. The rate of the reaction was studied in DFX and 9 other solvents in order to cover a range of polarities and obtain strong correlations (FIG. 2).

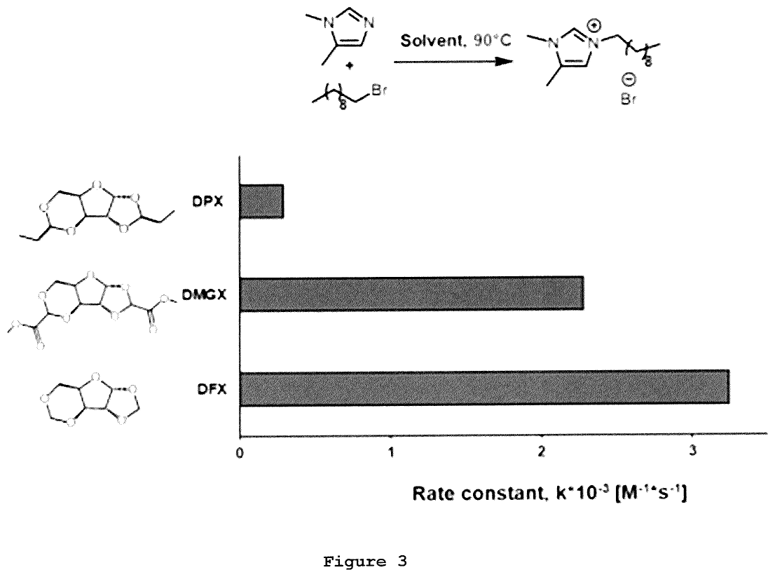

As shown in FIG. 3 the rate of this reaction appears to be proportional to the polarity of the solvent. This observation is consistent with the proposed mechanism of the Menshutkin reaction, where highly polar solvents significantly facilitate a charge separation at the transition state via favorable solute-solvent interactions, accelerating the whole process. This makes solvents like DMSO the best medium for heteroatom alkylations. However, typical highly polar solvents such as DMSO, DMF, NMP are toxic and associated with many environmental and sustainability concerns. In this regard, DFX could be one of the best alternatives for this type of reaction, outperforming some other polar aprotic solvents, including relatively new “green” ones such as -valerolactone (GVL) or cyclopentyl methyl ether (CPME).

The same reaction was tested in other compounds—Dipropylxylose (DPX, compound I wherein each of R₁ and R₁′ is ethyl and each of R₂ and R₂′ is hydrogen), Dibutylxylose (DBX, compound I wherein each of R₁ and R₁′ is propyl and each of R₂ and R₂′ is hydrogen), Diisobutylxylose (DIBX, compound I wherein each of R₁ and R₁′ is isopropyl and each of R₂ and R₂′ is hydrogen), Dimethylglyoxylate xylose (DMGX, VII).

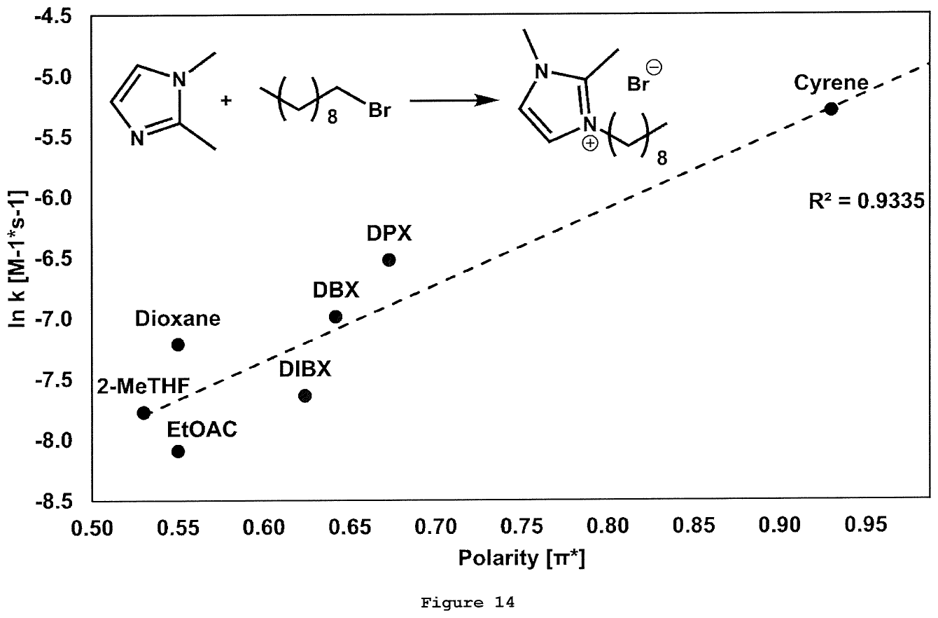

As depicted in FIG. 14 DPX, DBX, and DIBX solvents clearly outperformed traditional medium-polarity solvents (1,4-dioxane, 2-Me-THF, EtOAc), second only to much more polar solvents (FIG. 14).

For DMGX compounds, the procedure was the same as about but at 90° C. instead of 70° C. because melting point of DMGX derivative is about 82° C. For the comparison, the same protocol has been performed on DFX and DPX as well. The observed product was studied in all solvents and corresponding rate constants were calculated (FIG. 3). Since the reaction is very sensitive to solvent polarity, the slowest rate was found in DPX due to its lower polarity term in both Hansen and Kamlet-Taft models. DMGX was lower that DFX as expected as well.

### Example 4: Hydrogenation Reaction

The hydrogenation of cinnamaldehyde (CAL) was examined in a series of organic solvents at 70° C. over Pd/C catalyst in a 25 mL stainless steel Parr reactor. The reactor was loaded with cinnamaldehyde (CAL, 0.665 g, 5 mmol, 1.00 equiv.), Pd/C (1 wt %, 30 mg), and solvent (10 mL) and then sealed and pressurized with H₂ (40 bar). The reactor was heated up to 70° C. with stirring (600 rpm) and held at that temperature for the specified reaction time.

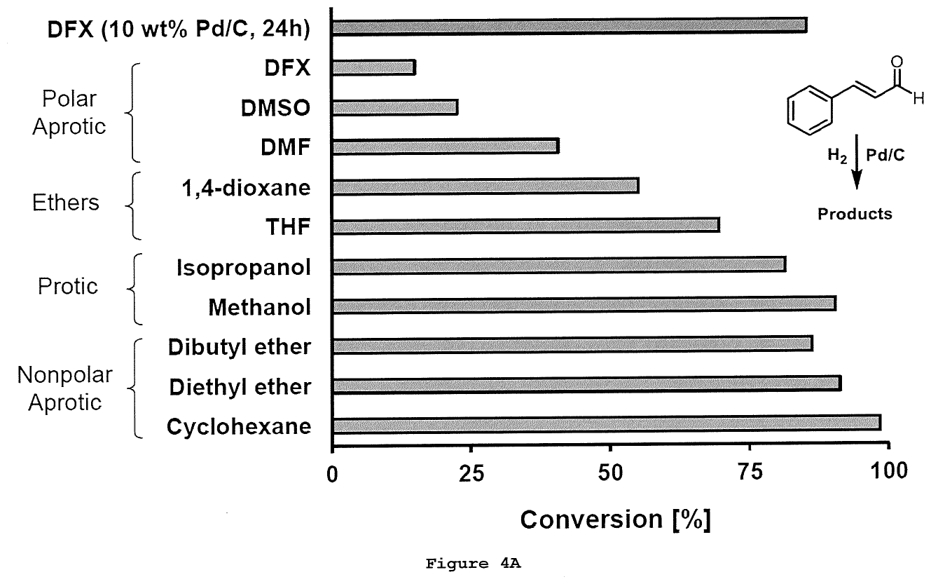

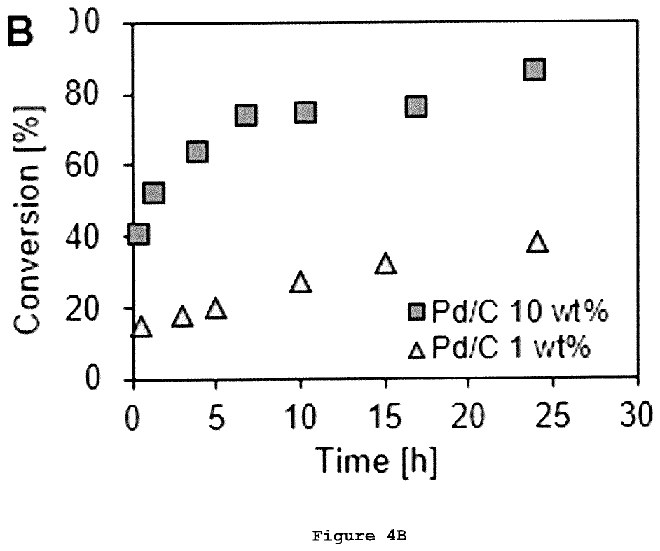

FIG. 4 (A) shows the conversion of cinnamaldehyde after hydrogenation in various solvents over 1 wt % Pd/C catalyst at 70° C., 30 min (if other not indicated) and FIG. 4(B) shows the conversion of DFX as a function of time at different Pd/C catalyst loadings. 81% and 90% conversion of CAL was achieved in isopropanol (IPA) and methanol (MeOH), respectively. Similarly, high conversions were also achieved in nonpolar aprotic solvents (Dibutyl and Diethyl ethers (DEE), and cyclohexane). Ethers with medium polarity (1,4-dioxane and THF) also facilitated the reaction, while conventional polar aprotic solvents (DMSO, DMF) and DFX were providing the slowest kinetics. Nevertheless, increasing catalyst loading to 10 wt % and letting the reaction proceed for 24 h gives 90% conversion in DFX making it a suitable solvent for this reaction if a cheap non-volatile bio-based solvent is required. In terms of selectivity, hydrocinnamaldehyde was the main product obtained in all tested solvents including DFX with a selectivity of 70% or higher. Hydrogenation in IPA and MeOH led to the formation of acetals, which is an undesirable feature of alcoholic solvents and limits their use for this reaction despite their promising conversions.

It is important to note that, DFX was stable under hydrogenation conditions (40 bar of H₂, 70° C., 24 h), thus it can be used in reactions requiring high pressure of H₂, elevated temperature, and/or long reaction time, which is a very desirable property for biomass-derived solvents.

### Example 5: Cross-Coupling (Heck) Reaction

Iodobenzene (0.69 mL, 6.00 mmol, 1.00 equiv.), methyl acrylate (0.54 mL, 6.00 mmol, 1.00 equiv.), triethylamine (0.84 mL, 6.00 mmol, 1.00 equiv.), and Pd(OAc)2 (0.1 mol %) were reacted at 90° C. in 5 ml of DFX and other solvents to compare their relative performance.

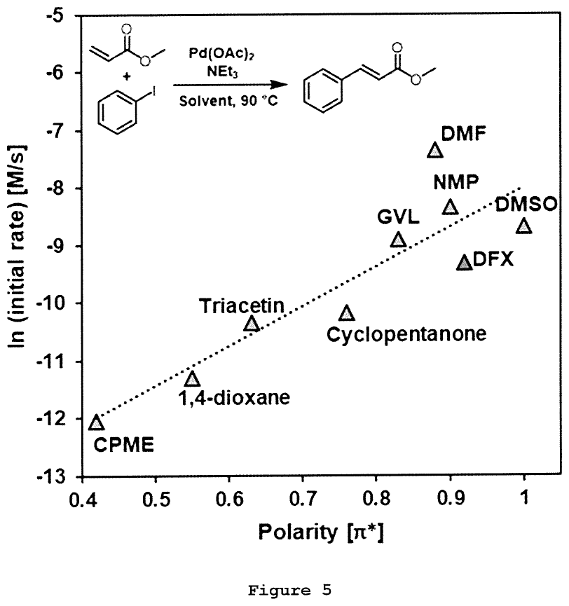

The kinetics of Mizoroki-Heck reaction demonstrated strong dependence on the polarity of the solvent used (FIG. 5). DFX provided relatively slow kinetics compared to the solvents of similar polarity. This fact can be related to the absence of any n bond in the structure of DFX, which usually coordinates over the forming palladium-carbon bond. Unsurprisingly, DMF as a solvent with the well-known coordination behavior provided the fastest kinetics, even though its polarity in terms of Kamlet-Abboud-Taft's π* values was not the highest among tested solvents.

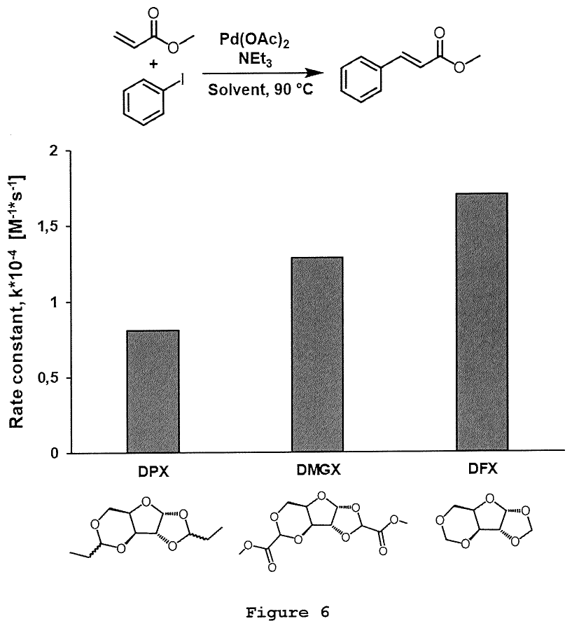

For DPX and DMGX in model Heck reaction, the following trend was observed: with increasing polarity, the rate of the reaction increases, and all molecules possessed the right properties to promote the reaction (FIG. 6).

A test of the same reaction protocol in DBX, DIBX (variations of compound (I)) showed accumulation of product with 15% conversion after 10 min of the reaction. For comparison, in 1,4-dioxane at the same reaction time no conversion has been observed.

### Example 6a: Physical and Solvation Properties of Diformylxylose

The most relevant solvent properties were measured for DFX and listed in Table 1a in comparison with other solvents. The boiling point of DFX was measured to be 237° C., which is close to the value of ethylene carbonate. Therefore, DFX can be separated from the reaction mixture by distillation, making its recycling more energy-consuming than for low-boiling solvents. However, high-boiling solvents are considered greener because human exposure risks and environmental impact (specifically aquatic toxicity) are reduced due to low volatility. DFX has a high melting point as well (48° C.). The density of DFX as determined experimentally is 1.35 g/mL at 50° C., which is close to the density of Sulfolane, Cyrene, and some chlorinated solvents. DFX has poor solubility in water, very similar to 2-Me-THE, which allows to easily recover DFX from water and use it in certain applications that require water-immiscible solvent and in water-organic extractions.

Flash point of 1 (DFX) was measured to be 137.5° C. In terms of safety, the greatest risk of injury is coming from solvents with a lower flash point, e.g. 2-Me-THF (−10° C.), CPME (−1° C.), NMP (86° C.), MTBE (−28° C.), Cyrene (108° C.), GVL (96.1° C.). In this regard, DFX is much more advantageous alternative.

TABLE 1a
Physical and Solvation properties of DFX and other
selected solvents.

| | Physical Properties | | | | | | | | | |
|---|---|---|---|---|---|---|---|---|---|---|
| | | | | Solubility | | | | | | |
| | | | Density, | in water, | | | | Hansen Solubility Parameters, | | |
| | Boiling | Melting | g/ml | g/100 g | Kamlet-Taft Parameters | | | MPa½ | | |
| Solvents | Point, ° C. | Poing, ° C. | (at 25° C.) | (at 25° C.) | α | β | Π* | δD | δP | δH |
| Diformylxylose | 237 | 48 | 1.35 | 13 | 0.00 | 0.82 | 0.92 | 17.9 | 9.0 | 7.60 |
| DMSO | 189 | 19 | 0.89 | ∞ | 0.00 | 0.74 | 1.00 | 18.4 | 16.4 | 10.2 |
| Ethylene carbonate | 238 | 35 | 1.32 | 26 | 0.00 | 0.32 | 0.99 | 18.0 | 21.7 | 5.10 |
| Sulfolane | 282 | 27.5 | 1.26 | ∞ | 0.00 | 0.39 | 0.98 | 17.8 | 17.4 | 8.70 |
| Cyrene | 155 | <−20 | 1.25 | ∞ | 0.00 | 0.61 | 0.93 | 18.9 | 12.7 | 7.10 |
| NMP | 202 | −24 | 1.25 | ∞ | 0.00 | 0.75 | 0.90 | 18.0 | 12.3 | 7.20 |
| GVL | 207 | −31 | 1.05 | ∞ | 0.00 | 0.60 | 0.83 | 15.5 | 4.70 | 6.60 |
| N-butylpyrrolidinone | 241 | <−75 | 0.96 | ∞ | 0.00 | 0.92 | 0.77 | 17.4 | 6.70 | 5.20 |
| 1,4-dioxane | 101 | 11.8 | 1.03 | ∞ | 0.00 | 0.37 | 0.55 | 17.3 | 4.30 | 8.40 |
| 2-Me—THF | 80 | −136 | 0.85 | 14 | 0.00 | 0.58 | 0.53 | 16.9 | 5.00 | 4.30 |

The solvatochromism of DFX (Table 1a) demonstrates that DEX is very polar since its value of measured Kamlet-Abboud-Taft solvatochromic parameter π* (0.92) is in a range of conventional highly polar aprotic solvents (e.g. DMSO, NMP). Hydrogen bond accepting ability (B) is also very high for DFX, which is due to the presence of 5 oxygen atoms in the structure, which can donate an electron pair. The parameter a was assigned to 0.00 as well as for other PAS because they cannot be hydrogen bond donors. Determination of the Kamlet-Abboud-Taft parameters was based on the absorption spectrum of two dyes (N,N-diethyl-4-nitroaniline and 4-nitroaniline) in different solvents, that allow calculating corresponding values using known procedures (P. G. Jessop, D. A. Jessop, D. Fu and L. Phan, Green Chem., 2012, 14, 1245-1259).

### Example 6b: Physical and Solvation Properties of DFX and Other Selected Compounds from Claim 13

Table 1b depicts Physical and Solvation properties of selected compounds from claim 13.

| | Melting | Boiling | Solubility | |
|---|---|---|---|---|
| Compound | point, ° C. | point, ° C. | in water | Density |
| 1 | 48 | 237 (at 760.00 mmHg) | 13 | 1.35 |
| | | 85 (at 0.015 mmHg) | | |
| 2 | 60 | n/m | n/m | n/m |
| 3 | 34 | 238 | 1.75 | 1.17 |
| 4 | 31 | 235 | <0.1 | 1.11 |
| 5 | 120 | n/m | ~0 | n/m |
| 11 | 43 | 121 | n/m | n/m |
| 14 | 83 | n/m | <0.1 | n/m |
| 24 | 24.5 | 234 | <0.1 | 1.15 |

n/m—not measured

Depending on the protection agent used to synthesize the compounds from claim 13, molecules with diverse physical properties that can be suitable for different applications can be produced. For example, compound 24 has the lowest melting point (24.5° C.) and can be used in reactions not requiring or avoiding high temperatures. Compounds 1, 3, 4, and 11 have a medium-range melting point, while 2, 5, and 14 are considered as high-melting compounds.

As for boiling points, the experimental values so far indicate little dependency on the type of aldehyde, resulting in boiling points remaining similar and high (234-238° C.). Although, ketone-protected compound 11 has the boiling point significantly lower (120° C.), which opens extra separation opportunities for this molecule. The advantage of having a low volatile solvent is a much lower risk of human exposure and reduced environmental impact, making the solvent more “green” compared to low-boiling analogs. The boiling points of the molecules are similar to some other “green” polar aprotic solvents such as Cygnet, N-butylpyrrolidinone, Ethylene carbonate, GVL, N-formylmorpholine. DMF.

The molecules are very close to being insoluble in water. This low solubility allows for these compounds to be easily separated from water mixtures and opens some applications for them when water-immiscible solvents are required.

The densities measured for the compounds are comparable to other solvents such as DMSO (1.1 g/cm3) and DCM (1.3 g/cm3).

Table 1c depicts Kamlet-Taft parameters measured for selected compounds from claim 13.

| | Kamlet-Abboud-Taft | | |
|---|---|---|---|
| Compound | α | β | π |
| 1 | 0 | 0.82 | 0.92 |
| 3 | 0 | 0.63 | 0.68 |
| 4 | 0 | 0.51 | 0.63 |
| 24 | 0 | 0.56 | 0.65 |
| THF | 0 | 0.55 | 0.58 |
| 1,4-Dioxane | 0 | 0.37 | 0.55 |
| 2-MeTHF | 0 | 0.58 | 0.53 |
| Cyclohexane | 0 | 0 | 0 |

According to measured Kamlet-Taft parameters, compounds 3, 4, and 24 are very close by solvation properties to medium-polarity solvents such as THE, 1,4-dioxane, 2-Me-THF. This means that the compounds can successfully complement the list of conventional ethers bringing new properties and, possibly, a safer profile. Moreover, the possibility of these compounds being produced sustainably from renewable sources makes them successful replacement candidates. Compound 1 is significantly more polar and can be a promising renewable alternative to toxic common polar aprotic solvents such as DMF, NMP, sulfolane, etc.

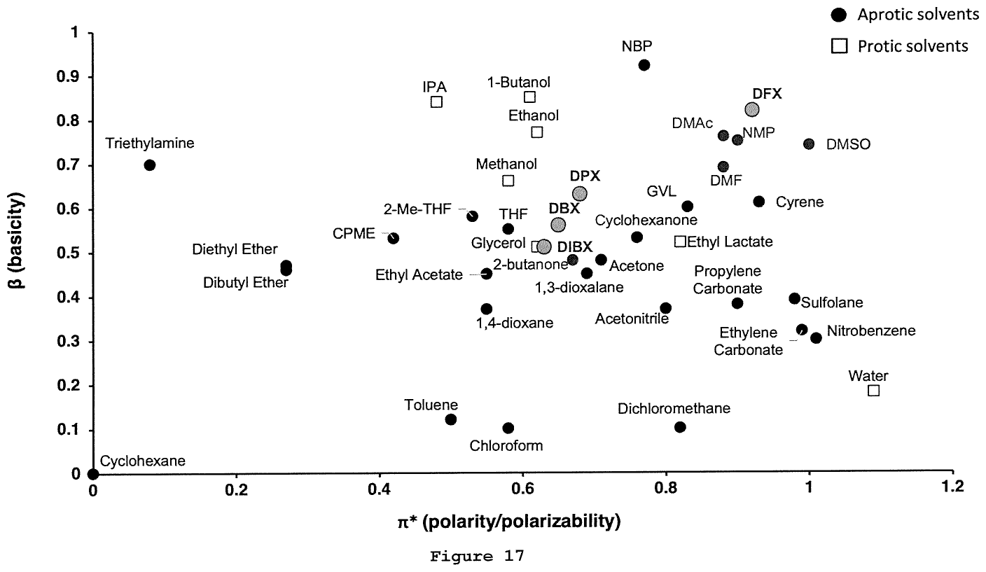

Based on the measured Kamlet-Abboud-Taft parameters, we established a two-dimensional solvent map to compare some of these compounds with other existing solvents in a parametric space (FIG. 17). Solvents, which are close to one another on the solvent map, are likely to have similar solvent properties. DEX occupies a unique space above conventional polar aprotic solvents, due to its high basicity. DPX, DBX, DIBX occupy region of less polar solvents. However, these areas of the solvent map are not yet populated by any known bio-based solvents or green solvents, which suggests that these compounds are novel solvents and promising candidates for the replacement of some of the highly toxic polar aprotic solvents such as NMP, DMF, DMAc, as well as medium-polarity ethers. The compounds can also have unique applications due to their high hydrogen bond accepting ability. Other compounds from claim 13, due to structural similarity, are likely to populate the same regions while having unique features (e.g. physical properties such as melting point, viscosity, vapour pressure, etc.) that can be wisely selected for specific application.

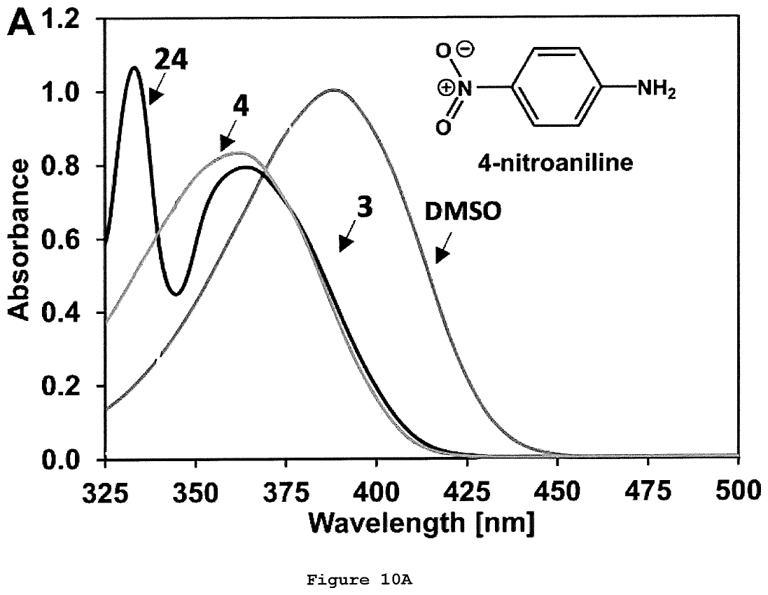

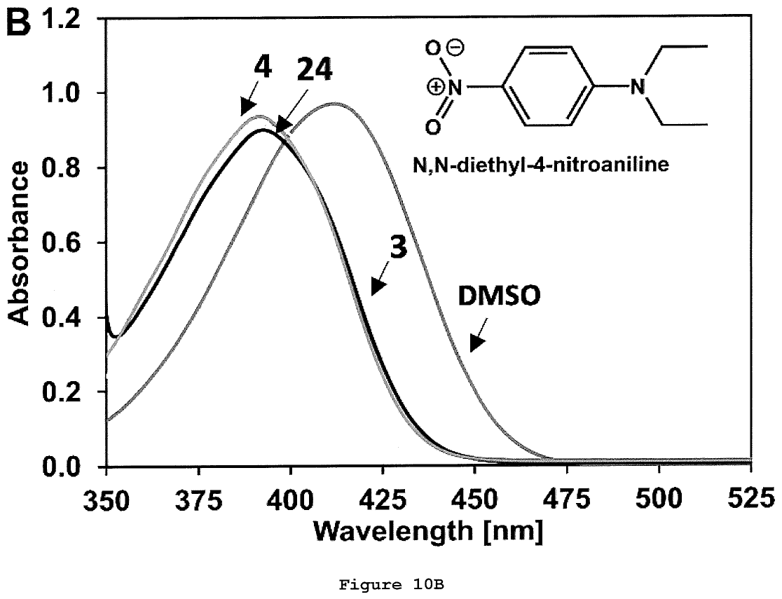

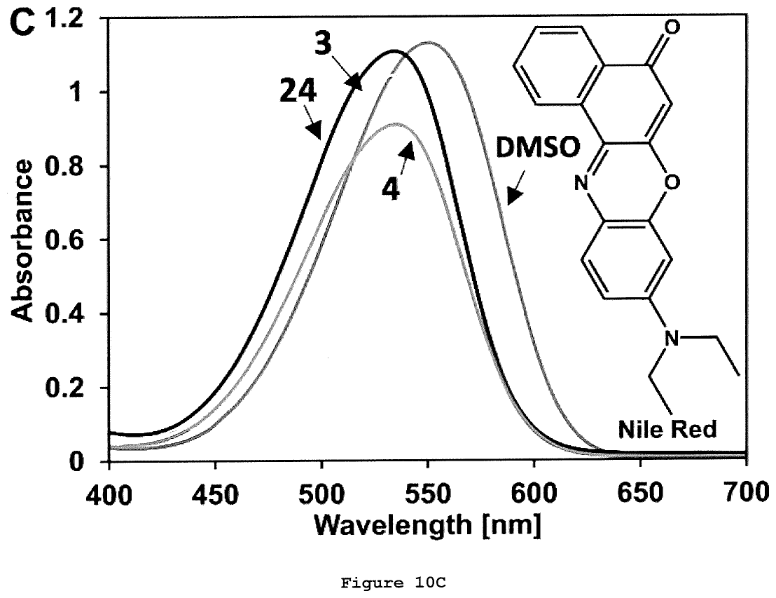

The absorbance spectra of 4-nitroaniline dye (cf. FIG. 10a), N,N-diethyl-4-nitroaniline dye (cf. FIG. 10b) as well as of Nile Red dye (cf. FIG. 10c) in the compounds 1, 3, 4 and 24 of claim 13 as well as in DMSO were measured. Nile Red absorbance is dependent on polarity and acidity, but as acidity is 0 in the case of aprotic solvents, the value is only polarity dependent. This differs from π*, which is both polarity and polarizability dependent. The maximum absorbance of Nile Red in 1, 3, 4 and 24 was measured to be 543, 536, 534.5 and 535 nm respectively. The value for 1 lies in the range between sulfolane (545 nm) and DMF (541 nm), which are two well-known aprotic solvents with high polarity. Values for 3, 4 and 24 are similar to conventional medium polarity aprotic solvents such as dichloromethane (535.2 nm) and acetonitrile (531.6 nm).

For the compounds 1 (Diformylxylose) and 24, miscibility with different organic solvents was tested to explore how this compound can be separated.

Table 1d shows Miscibility for Diformylxylose (compound 1) and compound 24 with “*” indicating—not miscible:

| Solvents | DFX | 24 |
|---|---|---|
| Acetonitrile | | |
| Anisole | | |
| Acetone | | |
| Acetonitrile | | |
| Chloroform | | |
| CPME | | |
| Cyclohexane | * | |
| DMF | | |
| DMSO | | |
| Dioxane | | |
| di-n-butyl ether | * | |
| Ethanol | | |
| Ethyl Acetate | | |
| Hexane | * | |
| Isopropanol | | |
| 2-Me-THF | | |
| THF | | |
| Toluene | | |
| Water | | * |

### Example 7: Toxicological Assessment

The toxicological assessment of DFX was performed using the Ames test to quickly determine if it has a mutagenic and carcinogenic potential.

An AMES-384 ISO test kit by EBPI Inc. (Canada) with two Salmonella typhimurium bacterial strains (TA100 with base-pair mutation hisG46 and TA98 with frameshift mutation hisD3052) was used. S9 liver homogenate from Aroclor 1254 Sprague-Dawley rats was used in a number of experiments as a source of mammal metabolic enzymes to expand the detection capabilities of the assay. For the test, DFX was dissolved in sterile water (100 mg/mL) and filtered through a 0.22 μm membrane filter. The maximum concentration of DFX in the exposure well was 80 mg/exposure well. Serial dilution of the sample was performed with a dilution factor of 2 (the minimum tested concentration was 2.5 mg/exposure well). 4-Nitroquinoline-N-oxide (4-NQO) was used as a positive control for the TA100 strain. 2-Nitrofluorene (2-NF) was used as a positive control for the TA98 strain. 2-Aminoanthracene (2-AA) was used as a positive control when a rat liver fraction S9 was added to the TA100 or TA98 strains. For the negative controls, the same quantity of sterile water was added to the wells as was added in the case of the DFX assay. Statistical analysis of the results included calculation of the baseline (the average response of negative control data and standard deviation), positive criteria for considering the testing compound as a mutagen (must be ≥2× baseline), and standard error of the mean. All calculations were conducted using an Excel spreadsheet provided by EBPI Inc.

The following procedure was applied in accordance with manufacturer's guidelines. Briefly, TA100 and TA98 bacterial strains were grown overnight and diluted until OD600 was 0.1 (for TA98) or 0.05 (for TA100). In a 24-well plate, samples containing grown bacteria and negative controls (water), positive controls, sterility controls, 5-serial dilutions of the DFX water (80, 40, 20, 10, 5, 2.5 mg/ml) were prepared and incubated at 37° C. for 100 min in a medium including sufficient histidine to initiate cell division. After the exposure, these samples were diluted in Reversion Media absent histidine in a second 24-well plate, then aliquoted into three 384-well plates and incubated at 37° C. for 48 h. After this, plats were scored visually: yellow wells indicated bacterial growth as they had undergone reverse mutation and could produce the histidine needed for their growth; purple wells indicated no reverse mutation of His+ biopathway in bacteria as they couldn't grow without histidine. The number of yellow wells was calculated and averaged to obtain the mean number of revertants. Baseline and positive criteria were then calculated in accordance with the manufacturer's guide.

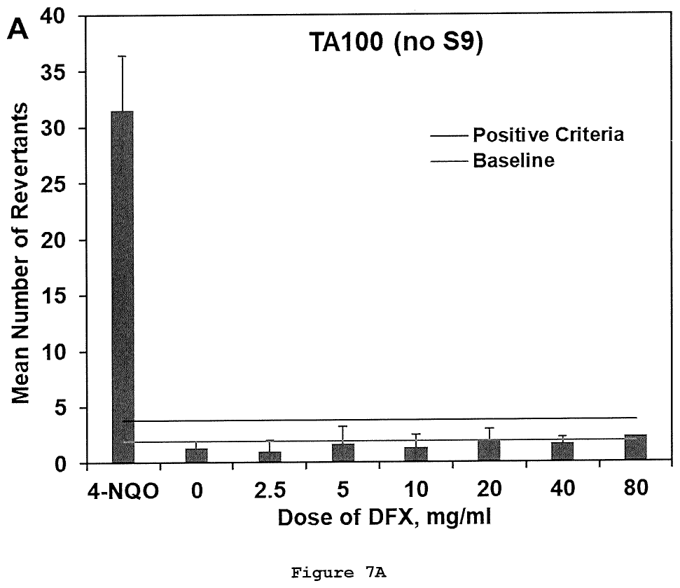

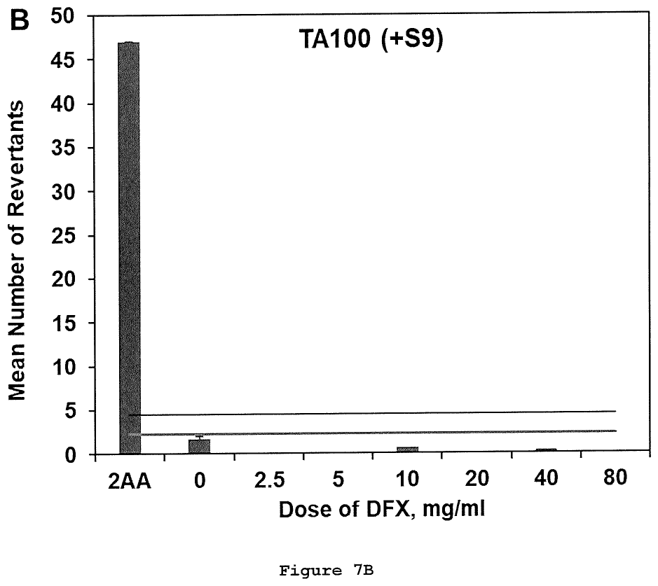

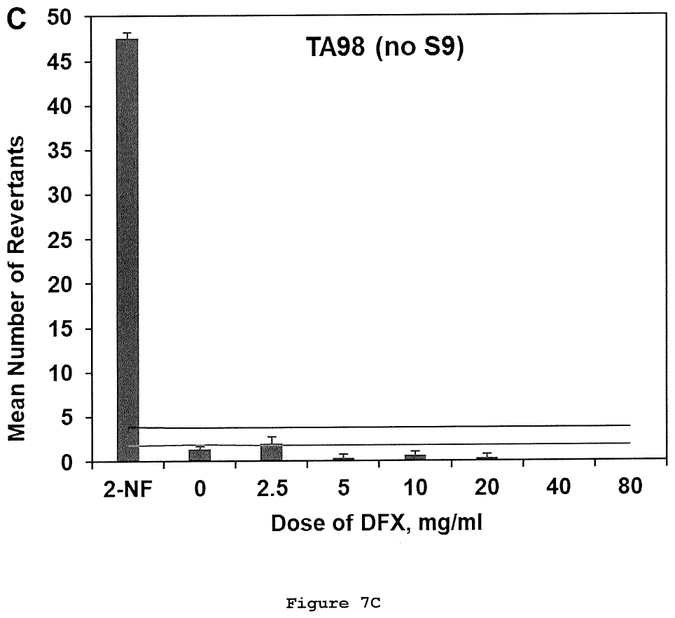

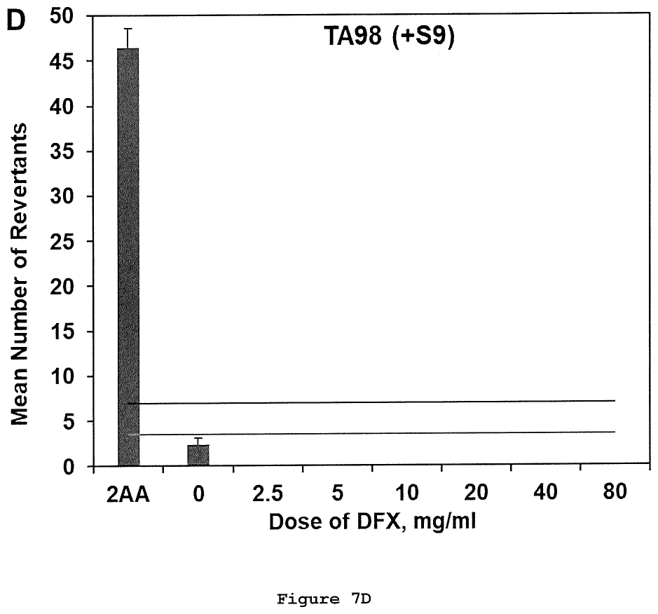

FIG. 7 shows the results of Ames test on DFX using Salmonella TA100 (A, B) and TA98 (C, D) strains in comparison with the positive control (4-Nitroquinoline-N-oxide (4-NQO) for TA100 strain; 2-Nitrofluorene (2-NF) for TA98 strain; 2-Aminoanthracene (2-AA) when S9 mix was added) and negative control (water) with S9 (B, D) and without (A, C). Error bars represent standard error of the mean. Green line is a calculated baseline, incorporating the average of negative control data and standard deviation. Red line is a positive criterion for considering the testing compound as a mutagen (≥2× baseline). It is clearly shown that DFX is unable to cause mutations both directly and indirectly, which makes it a promising molecule in terms of health and safety, although other extensive in vitro and in vivo tests are necessary to draw solid conclusions.

### Example 8: Biomass Processing

DFX is able to act as a solvent in formaldehyde-assisted biomass fractionation at several pre-treatment conditions (Table 2). The original procedure employs 1,4-dioxane as the main processing solvent, so it was used as a control. For pre-treatment in DFX, the yields of lignin and cellulose are slightly higher than in dioxane, possibly, due to the presence of solid degradation products (humins), formed because of interaction between acid and sugar derived DFX. The high boiling point of DFX (237° C.) allows to recovering pure DFX at the end of the procedure after evaporating other washing/precipitating solvents such as dioxane, methanol, water.

TABLE 2
Masses and yields of isolated fractions from the fractionation procedure.

| Solvent | Conditions | Lignin Yield | | Cellulose Yield | | DFX Yield/Recovery | |
|---|---|---|---|---|---|---|---|
| 1,4-dioxane | 95° C., 3.5 h | 0.44 g | (22.2%) | 0.88 g | (44%) | 0.61 g | (30.3%) |
| | 80° C., 5.5 h | 0.47 g | (23.7%) | 0.94 g | (47%) | 0.63 g | (31.2%) |
| | 70° C., 15 h | 0.53 g | (26.4%) | 0.91 g | (45.7%) | 0.62 g | (31%) |
| DFX | 95° C., 3.5 h | 0.74 g | (37%) | 1.18 g | (59%) | 9.17 g | (59.4%) |
| | 80° C., 5.5 h | 0.63 g | (31.7%) | 1.12 g | (56%) | 10.56 g | (69%) |
| | 70° C., 15 h | 0.64 g | (32%) | 0.92 g | (46%) | 12.57 g | (83%) |

The quality of the isolated lignin after pretreatment of Birch wood in DFX was assessed by determining the yield of aromatic monomers that could be produced after hydrogenolysis of the isolated lignin. The yield of monomers obtained after direct hydrogenolysis can be considered as an estimate of the theoretical monomer yield for a given biomass source. Lignin monomer identification and quantification were performed using GC-FID and GC-MS.

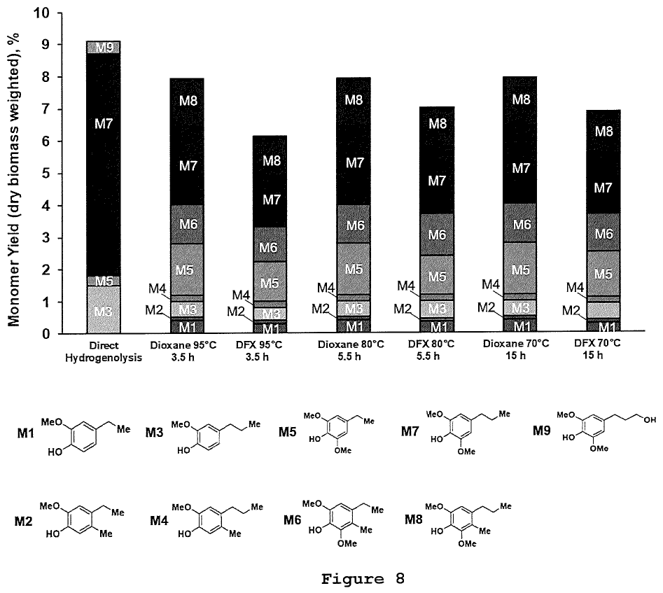

For pre-treatment in DFX, yields of aromatic monomers produced after hydrogenolysis of the isolated formaldehyde-stabilized lignin were in a range 70-80% (wt/wt) as compared with the direct hydrogenolysis of the biomass feedstock (2017 birch wood) (FIG. 8). Hydrogenolysis conditions were as following: catalyst Ru/C, 250° C., 40 bar of H₂, 3.5 h. Monomer yields are given based on dry biomass weight. The results indicate that DFX, like dioxane, can be used to efficiently isolate and purify lignin of good quality, which can be further upgraded in high yields. Overall, DFX is a promising green pre-treatment solvent for lignocellulosic biomass.

Solubility tests confirmed that DFX is a powerful solvent for the solubilization of lignin. The solubility of propionaldehyde-protected lignin in DFX is 0.633 g/g, which is higher than the corresponding value for 1,4-dioxane (0.450 g/g) and 2-Me-THF (0.330 g/g) after stirring the mixture for 1 h at 85° C.

Cellulose fibers were depolymerized after pretreatment in DFX in the same manner as for 1,4-dioxane and 2-Me-THF used as pretreatment solvents.

### Example 9: Stability of DFX

The stability of the compound (Ia) where R₁a=R₂a=R₁′a=R₂′a=H (DFX) was tested in acid and basic conditions.

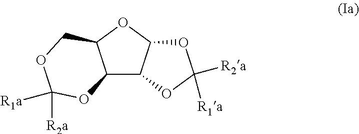

### Acidic Conditions

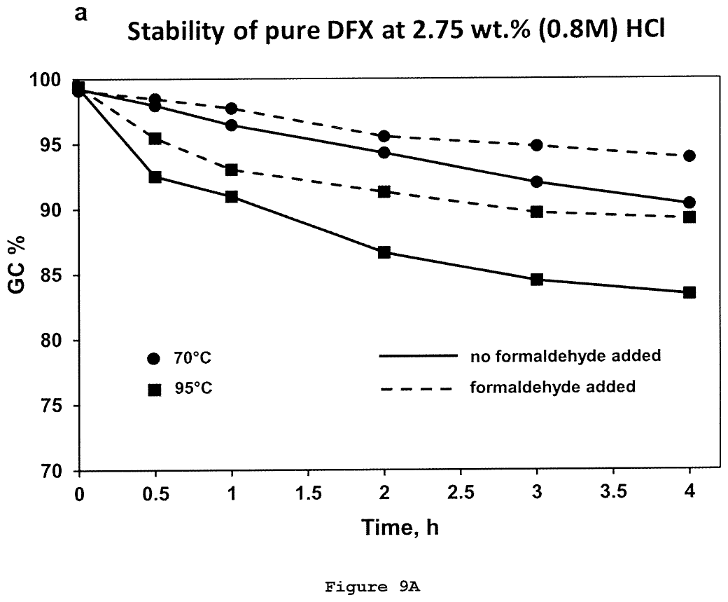

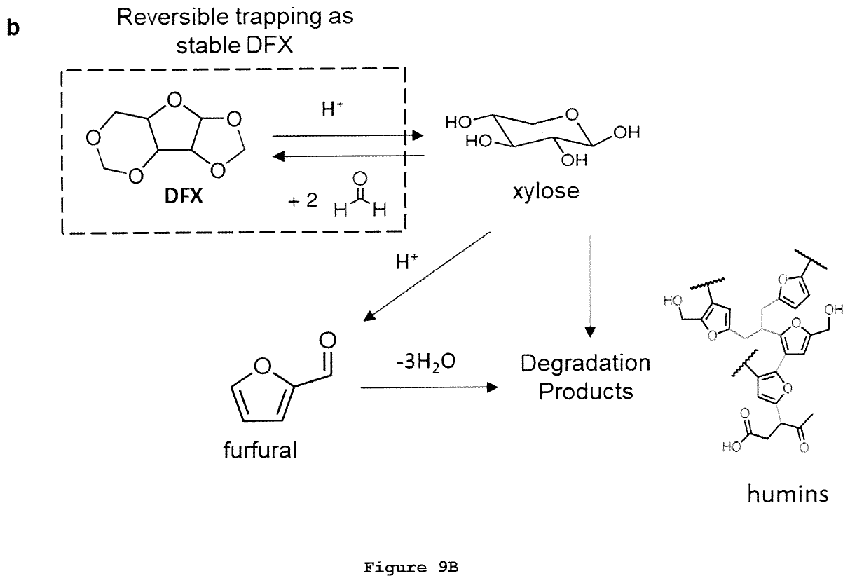

The stability of pure DFX under typical acidic conditions (0.8M HCl) was evaluated to determine if this sugar-derived molecule could be used without extensive degradation. The results demonstrated that at a temperature range between 70 to 95° C., more than 80% of DFX could be recovered (FIG. 9a). The 20% of loss was most likely caused by typical sugar degradation to products such as furfural, carboxylic acids, and polymeric insoluble humins. Interestingly, when adding formaldehyde, around 90% of DFX was stable likely due to a shift in equilibrium towards the reprotection of xylose (FIG. 9b). At the same conditions (95° C. or 70° C., 0.8M HCl) another novel commercially available carbohydrate-derived solvent Cyrene had undergone extensive degradation, forming a DFX remained dark solidified slurry after 15 min of reaction. In this regard, DFX is relatively stable under acidic conditions as its stability is superior to several alternatives.

In particular FIG. 9a shows the stability of pure diformylxylose at 2.75 wt. % (0.8M) HCl, 95 or 70° C. with formaldehyde added (straight line), or in the absence of it (dashed line), whereas FIG. 9b shows the DFX stabilization with formaldehyde.

Some variations of compound (I), specifically DPX, DBX, DIBX, Dineopentylxylose (DNPX, compound I wherein each of R₁ and R₁′ is tert-butyl and each of R₂ and R₂′ is hydrogen) also showed sensitivity to strong acids, which is visually observed as orange-colored solution compared to pure yellowish compound without acid.

### Basic Conditions

To assess DFX stability under basic conditions, we tested a series of organic and inorganic bases at 80° C. and 110° C. for maximum 48 h (Table 6). Visual color change compared to a blank solution of pure DFX indicated some reactions were occurring. After 0.5 h, 24 h, 48 h of exposure, the samples were injected in GC-FID and GC-MS and the area of DFX was compared with the one in blank sample using decane as the internal standard. As a result of this experiment, we identified some potential limitations of this emerging solvent. Specifically, inorganic bases such as K2CO3, NaOH, CsCO3 led to the highest extent of degradation (maximum 21% after 48 h). Triethylamine (NEt3) caused less than 5% of degradation, but led to the formation of dark solution as well as K2CO3, NaOH, CsCO3. Probably, impurities existing in DFX (95% pure) also contribute to a color change as they could interact with bases. Pyridine and KOAc as relatively weak bases didn't show any significant effect on stability of DEX.

TABLE 6
Stability of DFX under basic conditions

| | | | | | DFX degradation, | | |
|---|---|---|---|---|---|---|---|
| | Temperature, | Reaction (Y/N) | | | % | | |
| Base | ° C. | 0.5 h | 24 h | 48 h | 0.5 h | 24 h | 48 h |
| NEt₃ | 80 | Y | Y | Y | 3 | 4 | 5 |
| K₂CO₃ | 80 | Y | Y | Y | 2 | 3 | 7 |
| | 110 | Y | Y | Y | 2 | 5 | 13 |
| KOAc | 80 | N | N | N | 0 | 2 | 3 |
| | 110 | Y | Y | Y | 1 | 3 | 5 |
| Pyridine | 80 | N | N | N | 0 | 0 | 0 |
| | 110 | N | Y | Y | 0 | 3 | 6 |
| NaOH | 80 | Y | Y | Y | 0 | 9 | 17 |
| CsCO₃ | 110 | Y | Y | Y | 3 | 11 | 21 |
| | 80 | Y | Y | Y | 1 | 4 | 8 |
| | 110 | Y | Y | Y | 1 | 7 | 11 |

*Reaction (Y/N) means color change compared to blank solution.

### Thermal Conditions

DEX remained stable up to 210° C., according to TGA and DSC measurements. After 210° C. degradation with evaporation occurs.

Some variations of compound (I), specifically DPX, DBX, DIBX, Dineopentylxylose (DNPX, compound I wherein each of R₁ and R₁′ is tert-butyl and each of R₂ and R₂′ is hydrogen) as well as DMGX showed similar behaviour and were stable up to 200° C.

### Hydrogenation Conditions

DFX remained stable under the following hydrogenation conditions:
1. 40 bar of H₂, 70° C., 24 h
2. 40 bar of H₂, 250° C., 3 h

### Example 10: Preparing Deep Eutectic Solvents with Diformylxylose (DFX)

Deep eutectic solvents (DESs) are mixtures composed of a hydrogen bond donor and a hydrogen bond acceptor, resulting in a significantly suppressed melting point compared to those of the individual components. They are similar to ionic liquids but have some advantages to due their tunability, biodegradability and low cost.

DFX has a melting point of 48° C. and contains 5 oxygens in its structure and, therefore, is a strong hydrogen bond acceptor. Several hydrogen bond donors have been tested to check if they will form eutectic with DFX.

The following compounds mixed with DFX in 1:1 molar ratio were found to be stable liquids at room temperature and some of them are still liquids even at less than −18° C. (Table 3)

TABLE 3
Composition of melting points of DESs produced with DFX

| | Melting point | Melting point |
|---|---|---|
| | of the | of the mixture |
| Compound | compound, ° C. | with DFX, ° C. |
| Lactic acid | 17 | 4 < mp < 24 |
| Phenol | 40.5 | mp < −18 |
| Glyoxylic acid | 46.7 | 4 < mp < 24 |
| monohydrate | | |
| Resorcin | 110 | 4 < mp < 24 |
| Catechol | 105 | 4 < mp < 24 |
| Propyl guaiacol | 16 | mp < −18 |

These findings expand possible opportunities for DFX and its derivatives not only as simple solvents but also constituents for deep eutectic solvents. DESs composed of these molecules can be used in many applications. The physical properties of the liquids are dependent upon the hydrogen bond donor and can be easily tailored for specific applications. The two major application areas of DESs are metal processing (including metal electrodeposition, metal electropolishing, metal extraction and the processing of metal oxides) and synthesis media (e.g. alkylation, ionothermal synthesis, gas adsorption, biotransformations using microorganisms and reactions of sugars, cellulose, and starch, purifying and manufacturing biodiesel, etc).

We performed the alkylation reaction (the same as in the Example 3) in deep eutectic solvents (DES) composed of DFX and propyl guaiacol (DFX:PG) and DES composed of DFX and Phenol (DFX:PhOH) mixed in 1:1 molar ratio. We observed formation and accumulation of the reaction product with time and the reaction rate constant was quite similar to the one in CPME.

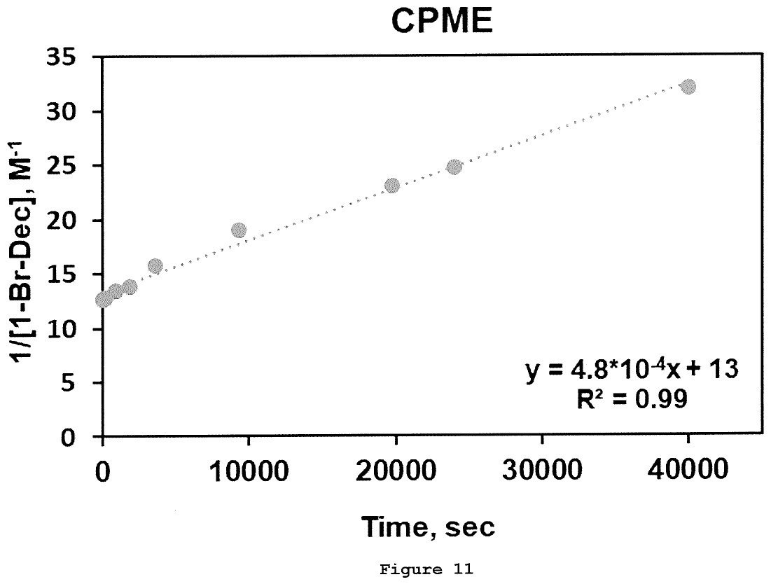

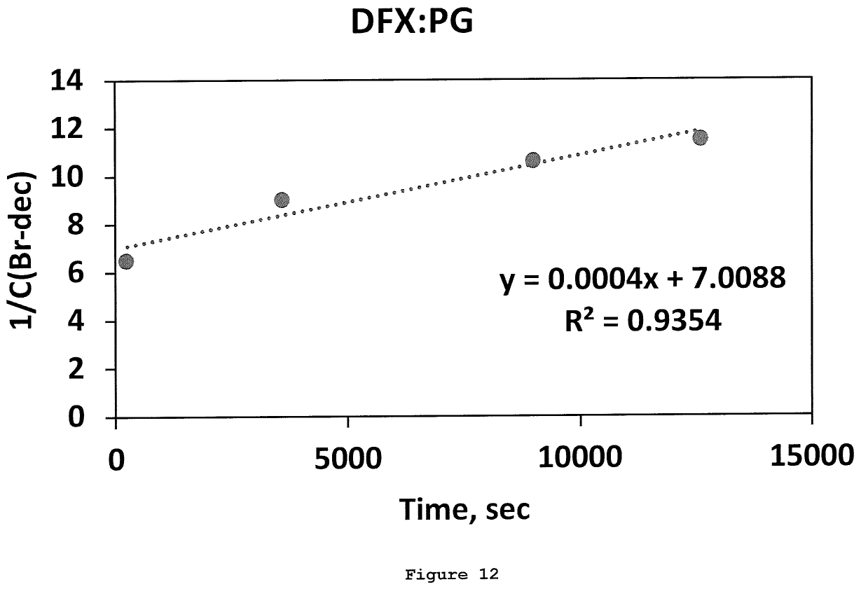

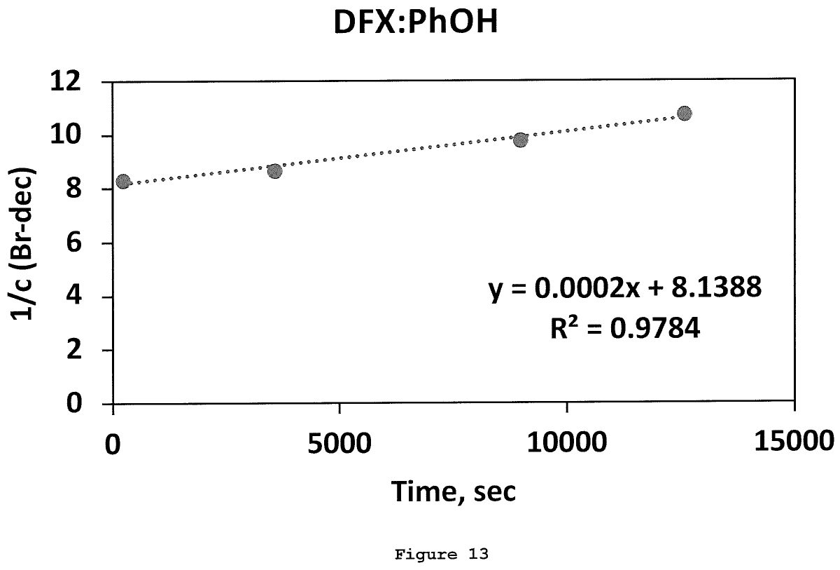

FIGS. 11, 12 and 13 show the plots of 1/concentration of 1-bromodecane as a function of time in different solvents, corresponding to second-order reaction kinetic. The slope represents a rate constant.

Since DESs contain hydrogen bond donors, they exhibit properties of protic solvents as well. Therefore applications for protic solvents are applicable to this class of solvents.

In order to explore DES composed of DFX better, the physical and solvation properties of the mixture of DFX and propyl guaiacol (DFX:PG) mixed in 1:1 molar ratio were measured.

Table 4 shows physical and solvation properties of DFX:PG (1:1 molar ratio).

| | | | | | | | Hansen | | |
|---|---|---|---|---|---|---|---|---|---|
| | Melting | | | | | | Solubility, | | |
| | point, | Boiling | Density, | Kamlet-Taft | | | MPa½ | | |
| Solvent | ° C. | point, ° C. | g/ml | α | β | Π* | δD | δP | δH |
| DFX:PG | <−18 | >250 | 1.2 | 0.59 | 0.34 | 18.7 | 18.7 | 9.69 | 9.68 |
| Acetone | −95 | 56 | 0.78 | 0.08 | 0.48 | 0.71 | 15.5 | 10.4 | 7 |
| Acetonitrile | −45 | 82 | 0.79 | 0.35 | 0.37 | 0.8 | 15.3 | 18 | 6.1 |

Based on the results, one can see that DFX:PG is a polar solvent with protic activity. Kamlet-Taft parameters suggest that DFX:PG is similar to acetonitrile, acetone, ethyl lactate. Hansen Parameters show that DFX:PG has some similarity to 1,3-dioxalane, NMP, Cyrene.

The Nile red value for DEX:PG is 553 nm, indicating similar chemical behavior to Methanol (550 nm) and Ethanolamine (557 nm).

In terms of thermal stability, no degradation of components was observed up to 250° C. (followed by refluxing and boiling).

Table 5 shows miscibility of DES-DEX:PG (1:1 molar ratio) with “*” indicating—not miscible.

| Solvents | DFX:PG |
|---|---|
| Acetone | |
| Acetonitrile | |
| CPME | |
| Cyclohexane | * |
| Dichloromethane | |
| DMF | |
| DMSO | |
| Dioxane | |
| Di-n-butyl ether | * |
| Ethanol | |
| Ethyl Acetate | |
| Hexane | * |
| Isopropanol | |
| 2-MeTHF | |
| THF | |
| Toluene | |
| Water | * |

### Example 10: Hansen Solubility Parameters

Each solvent can be characterized by three Hansen parameters, each generally measured in MPa 0.5. They are used to determine solubility of targets, estimate rate of reactions, etc.
δD—The energy from dispersion forces between molecules
δP—The energy from dipolar intermolecular force between molecules
δH—The energy from hydrogen bonds between molecules.

All three parameters were calculated for the molecules of Table 7 using HSPiP 5.3.02 Software (Table 7).

TABLE 7
Hansen Solubility Parameters of claim 13 and their
matching solvents from the Hansen Database

| | HSP | | | Closest match |
|---|---|---|---|---|
| Molecule | δD | δP | δH | solvent |
| 1 | 17.9 | 9.0 | 7.6 | Dimethyl isosorbide |
| 2 | 16.8 | 6.5 | 5.4 | Isophorone |
| 3 | 16.4 | 6.1 | 4.5 | Methyl isoamyl ketone |
| 4 | 16.2 | 4.9 | 3.2 | Ethyl butyl ketone |
| 5 | 15.5 | 3.8 | 2.5 | Di-n-butyl ether |
| 6 | 16.6 | 7.0 | 5.4 | Mesityl oxide |
| 7 | 16.3 | 6.8 | 4.8 | Mesityl oxide |
| 8 | 16.5 | 6.6 | 5.0 | Tri-n-butyl phosphate |
| 9 | 16.0 | 4.5 | 3.1 | Isopropyl palmitate |
| 10 | 15.6 | 3.6 | 2.5 | Di-n-pentyl-ether |
| 11 | 16.1 | 5.3 | 3.8 | Ethyl butyl ketone |
| 12 | 17.7 | 5.6 | 4.0 | Bromochloromethane |
| 13 | 18.5 | 10.8 | 8.7 | Tetramethylene |
| | | | | sulfoxide |
| 14 | 17.3 | 7.6 | 8.5 | Epichlorohydrin |
| 15 | 18.2 | 27.3 | 8.0 | Ethylene carbonate |
| 17 | 16.1 | 4.1 | 3.0 | Glycerol trioleate |
| 18 | 18.4 | 4.5 | 4.0 | Ethyl phenyl ether |
| 19 | 16.3 | 5.9 | 3.8 | Methyl amyl ketone |
| 20 | 15.7 | 5.5 | 3.2 | Methyl isoamyl ketone |
| 21 | 18.0 | 10.4 | 16.7 | Glycerol formal |
| 22 | 16.9 | 7.9 | 6.4 | Dichloromethane |
| 23 | 17.2 | 5.4 | 3.1 | Bromochloromethane |
| 24 | 16.3 | 5.2 | 3.8 | Ethyl butyl ketone |
| DMSO | 18.4 | 16.4 | 10.2 | DMSO |
| DMF | 17.4 | 13.7 | 11.3 | DMF |
| NMP | 18.0 | 12.3 | 7.2 | NMP |
| MeCN | 15.3 | 18.0 | 6.1 | MeCN |
| THF | 16.8 | 5.7 | 8.0 | THF |

Based on determined HSP, solvents 1-24 were placed in a 3D space, called Hansen space with three coordinates D, P, H (values from Table 4). This allows for the calculation of a distance between new molecules and already known compounds from the database to determine their chemically similar counterparts. Solvents that are the closest match by properties to the compounds 1-24 are likely to have similar applications and performance. However, in the case of 1-24 they could be produced sustainably from a renewable source and they probably could have a safer profile as they are derived from natural carbohydrates (D-xylose).

Table 8 depicts Hansen Solubility Parameters for variations of compound (III) of claim 1.

| | | HSP | | |
|---|---|---|---|---|
| Compound | Structure | δD | δP | δH |
| 25 | 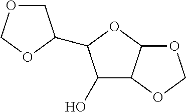 [SMILES: OC1C(C2COCO2)OC2OCOC21] | 18 | 10 | 12.1 |
| 26 | 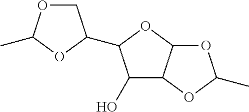 | 17.1 | 7.6 | 9.2 |
| 27 | 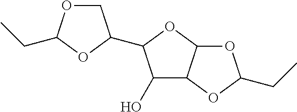 [SMILES: CCC1OCC(C2OC3OC(CC)OC3C2O)O1] | 16.6 | 7.2 | 7.9 |
| 28 | 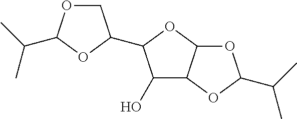 [SMILES: CC(C)C1OCC(C2OC3OC(C(C)C)OC3C2O)O1] | 16.5 | 6.1 | 6.2 |
| 29 | 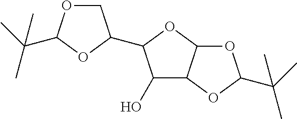 [SMILES: CC(C)(C)C1OCC(C2OC3OC(C(C)(C)C)OC3C2O)O1] | 15.7 | 4.9 | 0.1 |
| 30 | 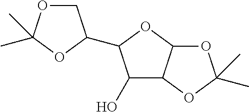 | 16.3 | 6.4 | 7.7 |
| 31 | 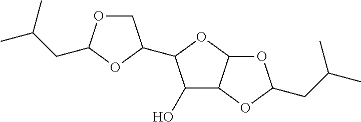 [SMILES: CC(C)CC1OCC(C2OC3OC(CC(C)C)OC3C2O)O1] | 16.2 | 5.5 | 5.8 |
| 32 | 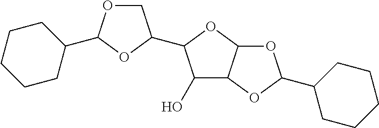 [SMILES: OC1C(C2COC(C3CCCCC3)O2)OC2OC(C3CCCCC3)OC21] | 17.4 | 5.5 | 6.5 |
| 33 | 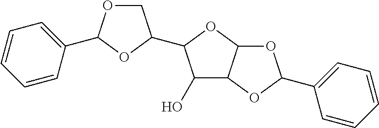 [SMILES: OC1C(C2COC(c3ccccc3)O2)OC2OC(c3ccccc3)OC21] | 18.4 | 5.4 | 6.8 |
| 34 | 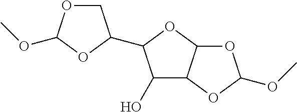 | 17.1 | 8.9 | 9.7 |
| 35 | 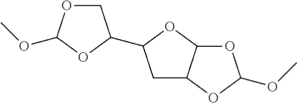 | 17 | 7.7 | 6.2 |
| 36 | 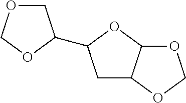 [SMILES: C1OCC(C2CC3OCOC3O2)O1] | 17.7 | 8.6 | 7.2 |
| 37 | 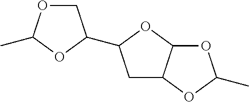 | 16.9 | 6.3 | 5.2 |
| 38 | 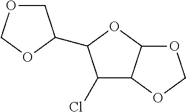 [SMILES: ClC1C(C2COCO2)OC2OCOC21] | 18.1 | 11.3 | 6.8 |
| 39 | 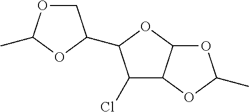 | 17.1 | 8.8 | 4.9 |
| 40 | 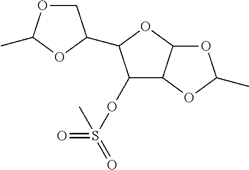 | 17.1 | 10.5 | 5.8 |
| 41 | 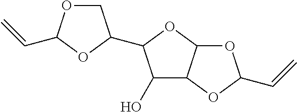 [SMILES: C=CC1OCC(C2OC3OC(C=C)OC3C2O)O1] | 16.9 | 6.3 | 7.9 |
| 42 | 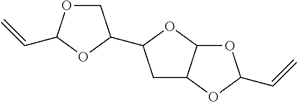 [SMILES: C=CC1OCC(C2CC3OC(C=C)OC3O2)O1] | 16.8 | 5.2 | 4 |
| 43 | 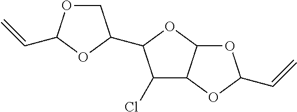 [SMILES: C=CC1OCC(C2OC3OC(C=C)OC3C2Cl)O1] | 16.9 | 7.6 | 3.8 |
| 44 | 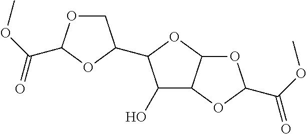 [SMILES: COC(=O)C1OCC(C2OC3OC(C(=O)OC)OC3C2O)O1] | 17.4 | 8.4 | 11 |
| 45 | 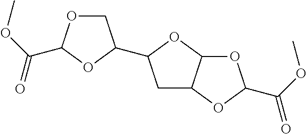 [SMILES: COC(=O)C1OCC(C2CC3OC(C(=O)OC)OC3O2)O1] | 17.5 | 7.4 | 8.1 |
| 46 | 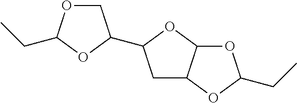 [SMILES: CCC1OCC(C2CC3OC(CC)OC3O2)O1] | 16.5 | 5.9 | 4.4 |
| 47 | 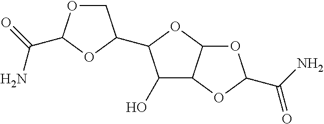 [SMILES: NC(=O)C1OCC(C2OC3OC(C(N)=O)OC3C2O)O1] | 18.3 | 18.8 | 17.2 |
| 48 | 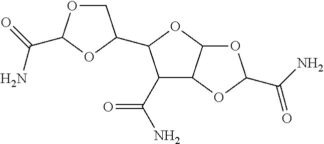 [SMILES: NC(=O)C1OCC(C2OC3OC(C(N)=O)OC3C2C(N)=O)O1] | 18.5 | 17.8 | 14.3 |
| 49 | 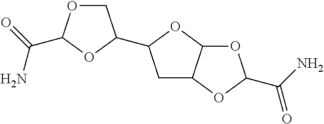 [SMILES: NC(=O)C1OCC(C2CC3OC(C(N)=O)OC3O2)O1] | 18.4 | 21.5 | 17.3 |
| 50 | 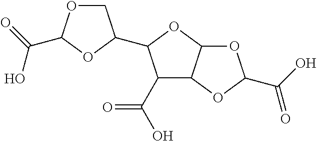 [SMILES: O=C(O)C1OCC(C2OC3OC(C(=O)O)OC3C2C(=O)O)O1] | 17.9 | 10.5 | 18.7 |
| 51 | 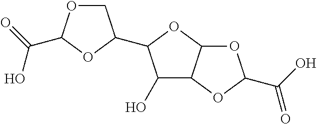 [SMILES: O=C(O)C1OCC(C2OC3OC(C(=O)O)OC3C2O)O1] | 17.9 | 11.2 | 18.3 |
| 52 | 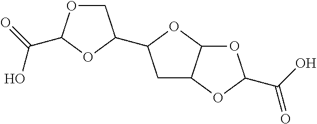 [SMILES: O=C(O)C1OCC(C2CC3OC(C(=O)O)OC3O2)O1] | 18.2 | 10.1 | 15.8 |

Table 9 depicts Hansen Solubility Parameters for variations of compounds (IV) (V) and (VI) of claim 1

| | | HSP | | |
|---|---|---|---|---|
| Compound | Structure | δD | δP | δH |
| 53 | 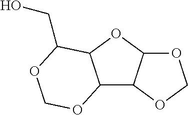 [SMILES: OCC1OCOC2C1OC1OCOC12] | 17.9 | 9.9 | 10.6 |
| 54 | 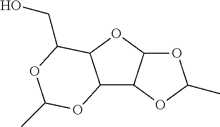 [SMILES: CC1OC2OC3C(CO)OC(C)OC3C2O1] | 16.6 | 7.5 | 8.2 |
| 55 | 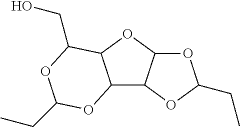 [SMILES: CCC1OC2OC3C(CO)OC(CC)OC3C2O1] | 16.2 | 7.1 | 7.1 |
| 56 | 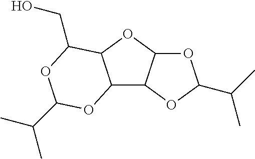 [SMILES: CC(C)C1OC2OC3C(CO)OC(C(C)C)OC3C2O1] | 16.1 | 6 | 5.2 |
| 57 | 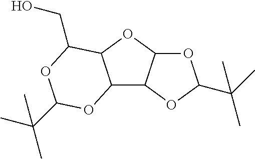 [SMILES: CC(C)(C)C1OC2OC3C(CO)OC(C(C)(C)C)OC3C2O1] | 15.4 | 4.8 | 4.8 |
| 58 | 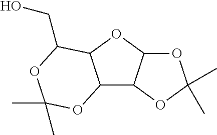 | 16 | 6.3 | 6.5 |
| 59 | 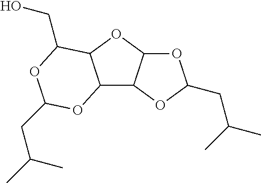 [SMILES: CC(C)CC1OC2OC3C(CO)OC(CC(C)C)OC3C2O1] | 15.8 | 5.4 | 5.3 |
| 60 | 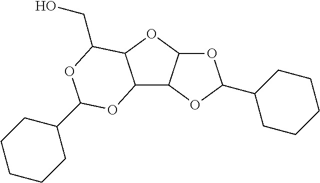 [SMILES: OCC1OC(C2CCCCC2)OC2C1OC1OC(C3CCCCC3)OC12] | 17.3 | 5.4 | 5.7 |
| 61 | 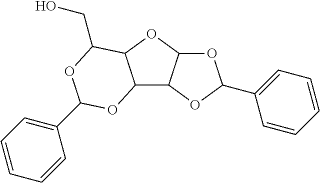 [SMILES: OCC1OC(c2ccccc2)OC2C1OC1OC(c3ccccc3)OC12] | 18.1 | 5.3 | 6.2 |
| 62 | 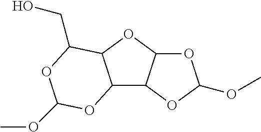 | 16.6 | 8.8 | 8.4 |
| 63 | 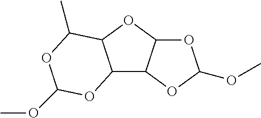 | 16.3 | 7 | 5.6 |
| 64 | 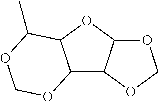 | 17.3 | 7.6 | 6.3 |
| 65 | 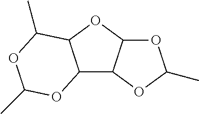 | 16.3 | 5.6 | 4.6 |
| 66 | 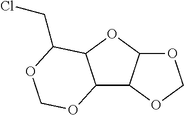 [SMILES: ClCC1OCOC2C1OC1OCOC12] | 17.9 | 9.5 | 6.7 |
| 67 | 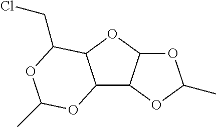 [SMILES: CC1OC2OC3C(CCl)OC(C)OC3C2O1] | 16.9 | 7.3 | 4.9 |
| 68 | 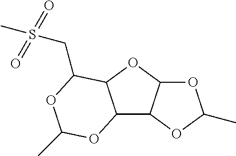 | 16.8 | 12.3 | 6.6 |
| 69 | 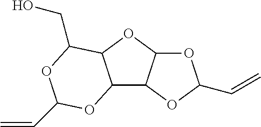 [SMILES: C=CC1OC2OC3C(CO)OC(C=C)OC3C2O1] | 16.5 | 6.2 | 6.6 |
| 70 | 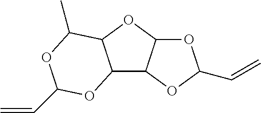 [SMILES: C=CC1OC2OC3C(C)OC(C=C)OC3C2O1] | 16.2 | 4.5 | 3.5 |
| 71 | 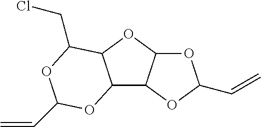 [SMILES: C=CC1OC2OC3C(CCl)OC(C=C)OC3C2O1] | 16.7 | 6.2 | 3.8 |
| 72 | 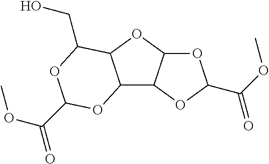 [SMILES: COC(=O)C1OC2OC3C(CO)OC(C(=O)OC)OC3C2O1] | 16.9 | 8.3 | 9.5 |
| 73 | 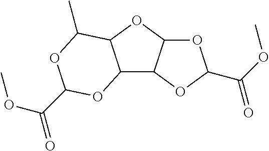 [SMILES: COC(=O)C1OC2OC3C(C)OC(C(=O)OC)OC3C2O1] | 16.7 | 6.6 | 7.5 |
| 74 | 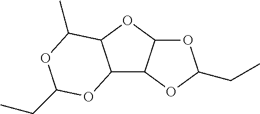 [SMILES: CCC1OC2OC3C(C)OC(CC)OC3C2O1] | 15.9 | 5.3 | 3.9 |
| 75 | 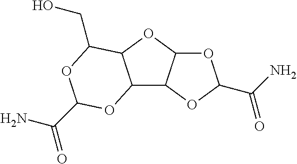 [SMILES: NC(=O)C1OC2OC3C(CO)OC(C(N)=O)OC3C2O1] | 17.7 | 18.7 | 15.9 |
| 76 | 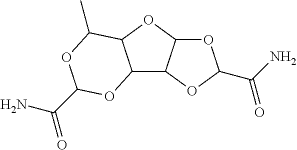 [SMILES: CC1OC(C(N)=O)OC2C1OC1OC(C(N)=O)OC12] | 17.5 | 16.8 | 13.6 |
| 77 | 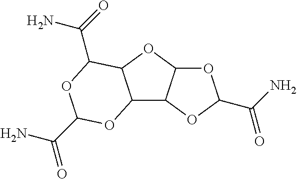 [SMILES: NC(=O)C1OC2OC3C(C(N)=O)OC(C(N)=O)OC3C2O1] | 18 | 22.3 | 18.2 |
| 78 | 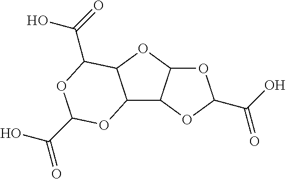 [SMILES: O=C(O)C1OC2OC3C(C(=O)O)OC(C(=O)O)OC3C2O1] | 17.4 | 10.9 | 19.8 |
| 79 | 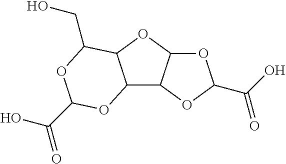 [SMILES: O=C(O)C1OC2OC3C(CO)OC(C(=O)O)OC3C2O1] | 17.3 | 11 | 16.9 |
| 80 | 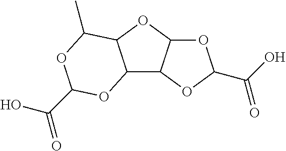 [SMILES: CC1OC(C(=O)O)OC2C1OC1OC(C(=O)O)OC12] | 17.3 | 9.2 | 15 |
| 81 | 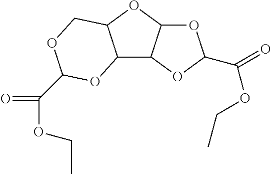 [SMILES: CCOC(=O)C1OCC2OC3OC(C(=O)OCC)OC3C2O1] | 16.8 | 7.3 | 7.4 |
| 82 | 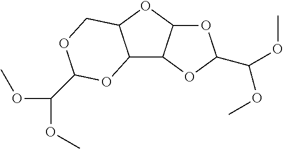 | 16.8 | 7.1 | 6.0 |

1. Use of a compound of the general formula (I), (II), (III) (VI), (V) or (VI) 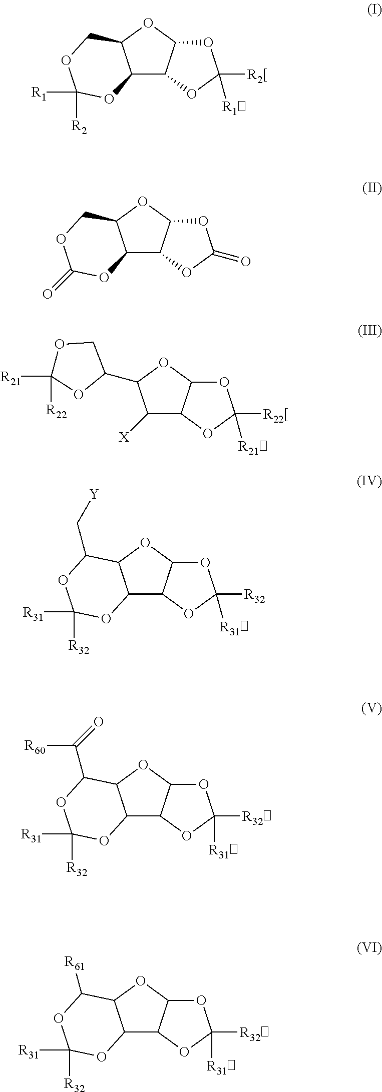
wherein R₁ and R₁′, R₂₁ and R₂₁′, R₃₁ and R₃₁′ are the same and are hydrogen, a linear or branched C₁ to C₁₈ alkyl, a linear or branched C₂ to C₁₈ alkenyl, a linear or branched C to C₁₀ alkoxy, a linear or branched C₁ to C₉ alkanoyloxy, a linear or branched C₁ to C₉ alkoxycarbonyl, aminocarbonyl, hydroxycarbonyl, a linear or branched C₁ to C₉ alkoxyalkyl, an unsubstituted cycloalkyl, a linear or branched C₁ to C₆ alkyl substituted cycloalkyl, preferably an unsubstituted cyclohexyl, an unsubstituted C₆ to C₁₂ aryl, a linear or branched C₁ to C₄ alkyl substituted C₆ to C₁₂ aryl, or C₇ to C₁₂ aralkyl,
R₂ and R₂′, R₂₂ and R₂₂′, R₃₂ and R₃₂′ are the same and are hydrogen, a linear or branched C₁ to C₁₈ alkyl, a linear or branched C₁ to C₉ alkoxyalkyl, or
R₂ and R₂′ form together with R₁ and R₁′ respectively, R₂₂ and R₂₂′ form together with R₂₁ and R₂₁′ respectively, R₃₂ and R₃₂′ form together with R₁ and R₁′ respectively, a 5 or 6 membered unsaturated or saturated carbocyclic ring optionally comprising 1 or 2 oxygen atoms,
X is selected from the group consisting of hydrogen, fluoro, chloro, bromo, iodo, alkoxycarbonyl, aminocarbonyl, hydroxycarbonyl, an alkanoyloxy, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, and a linear or branched C₁ to C₁₈ alkyl,
Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, a linear or branched C₁ to C₉ alkanoyloxy, and a linear or branched C₁ to C₉ alkylcarbonyl, and
R₆₀ is selected from the group consisting of hydrogen, hydroxy and C₁ to C₁₈ alkoxy, and
R₆₁ is selected from the group consisting of hydrogen, hydroxy, C₁ to C₁₀ alkylsulfonyl-C₁ to C₅ and C₁ to C₁₈ alkyl
as solvent for chemical reactions, for recrystallizations or extractions.

2. Use of compound (I), (II), (III), (IV) according to claim 1 as polar aprotic solvent, wherein compounds (I) and (II) are as defined in claim 1 and in compound (III) and in compound (IV) R₅₀ is not hydrogen.

3. Use of the compounds (III) and (IV) according to claim 1 as polar protic solvent, wherein the compounds (III) and (IV) R₅₀ is hydrogen or compound (V).

4. Use according to claim 1, wherein the solvent is used for chemical reactions.

5. Use according to claim 1, wherein the chemical reaction involves at least two starting compounds.

6. Use according to claim 1, wherein the solvent is used for a chemical reaction in the presence of an acid or a base, preferably a strong acid or strong base.

7. Use according to claim 1, wherein each of R₂ and R₂′ is hydrogen.

8. Use according to claim 1, wherein R₁ and R₁′ are hydrogen or a linear C₁ to C₁₈ alkyl, a linear C₂ to C₁₈ alkenyl, a linear C₁ to C₁₈ alkoxy, a linear or branched C₁ to C₉ alkanoyloxy, a linear C₁ to C₉ alkoxycarbonyl or a linear C₁ to C₉ alkoxyalkyl, preferably a linear C₁ to C₉ alkoxycarbonyl.

9. Use according to claim 1, wherein R₁ and R₁′ are a branched C₁ to C₁₈ alkyl, a branched C₁ to C₉ alkoxyalkyl, and wherein R₂ and R₂′ are the same and both hydrogen.

10. Use according to claim 1, wherein the chemical reaction is selected from the group consisting of the Heck reaction, alkylation reaction, hydrogenation reaction, Ullmann reaction or Ullmann coupling, Wurtz reaction, Grignard reaction, Gomberg-Bachmann reaction, Castro-Stephens coupling, Corey-House synthesis, Cassar reaction, Kumada coupling, Sonogashira coupling, Negishi coupling, Stille cross coupling, Suzuki reaction, Hiyama coupling, Buchwald-Hartwig reaction, Fukuyama coupling, Liebeskind-Srogl coupling, Solid-Phase Peptide Synthesis (SPPS), nucleophilic substitution, amidation, esterification, biginelli reaction, carbonyl addition, aza-Michael addition, Krapcho dealkoxycarbonylation, Boulton-Katritzky rearrangement, Michael addition and Beckmann rearrangement, and dehydrohalide coupling.

11. Use according to claim 1, wherein the chemical reaction is selected from the group consisting of Heck reaction, alkylation reaction, aldehyde-assisted biomass fractionation, and hydrogenation reaction.

12. Use according to claim 3, wherein the chemical reaction is selected from the group consisting of the reduction of nitro compounds, hydrogenation, halogenation, synthesis of ionic liquids, nucleophilic substitution with SN1 mechanism, elimination reaction with E1 mechanism and E2 mechanism, synthesis via SnAr mechanism, extractions of bioactive compounds and extractions of any compounds from biological sources.

13. Use according to claim 1, wherein the compound of formula (I), (II), (III), (IV), (V) or (VI) is selected from the group consisting of

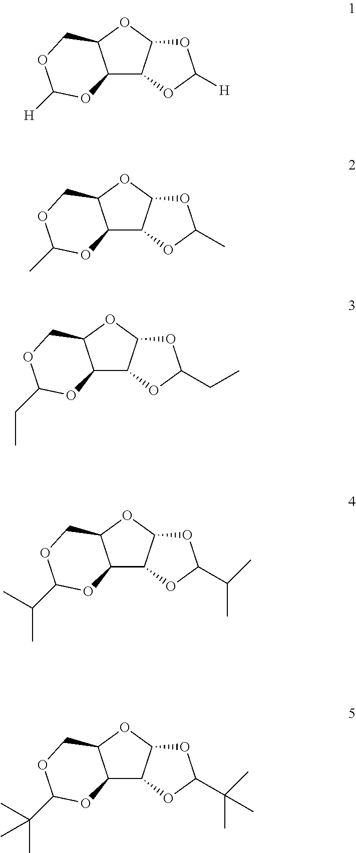

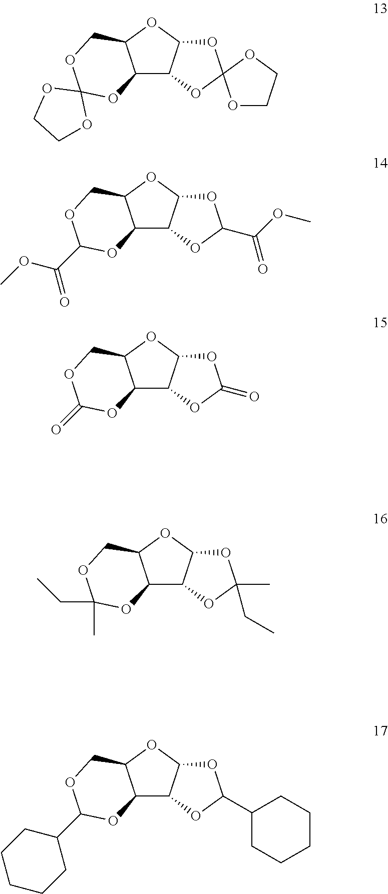

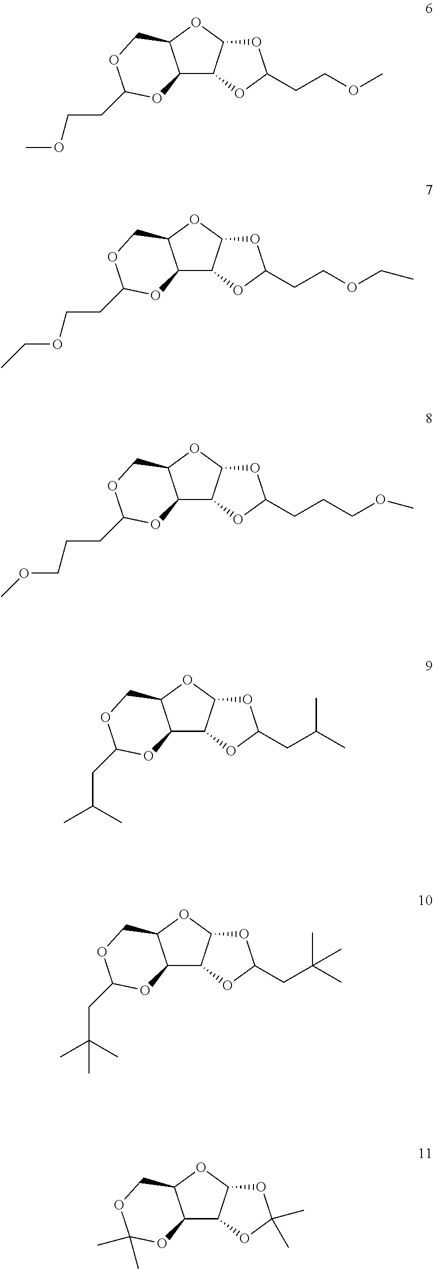

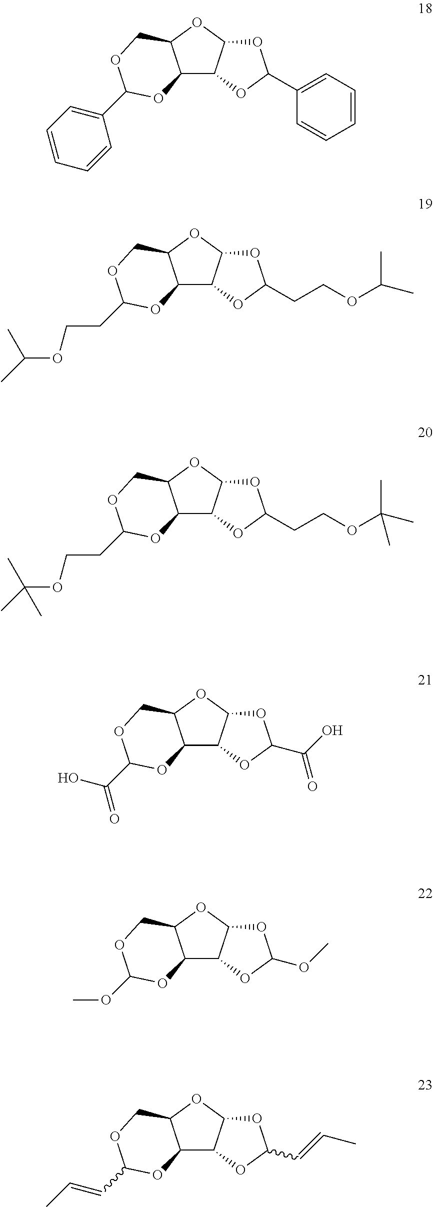

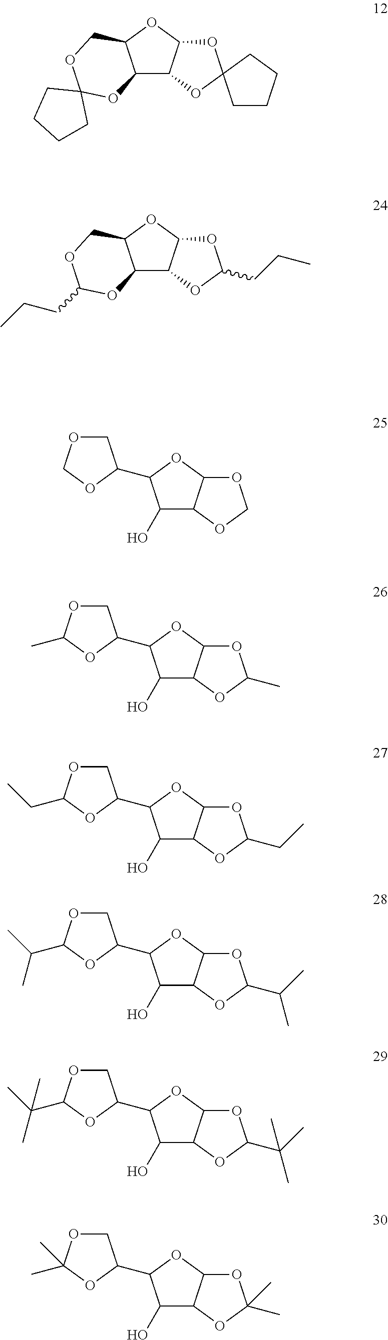

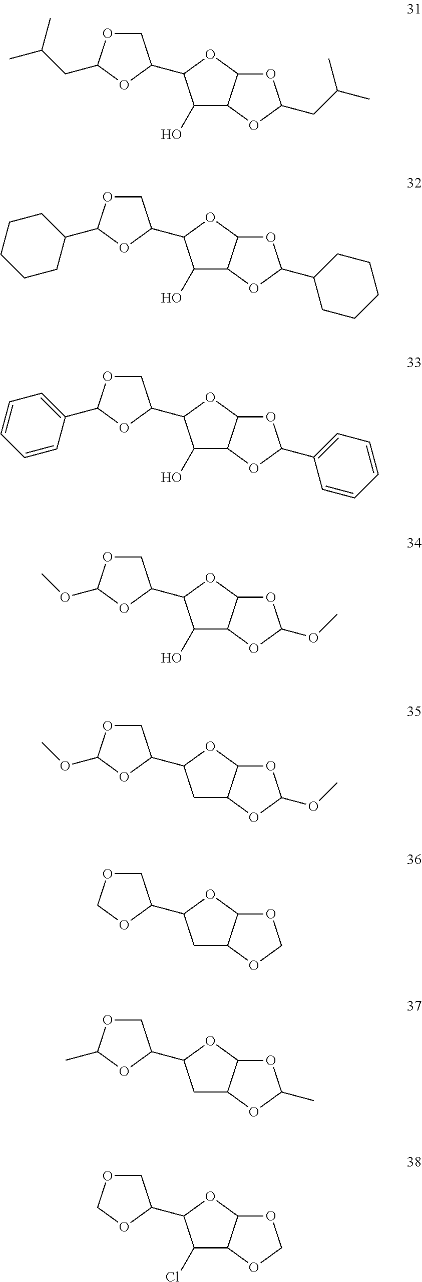

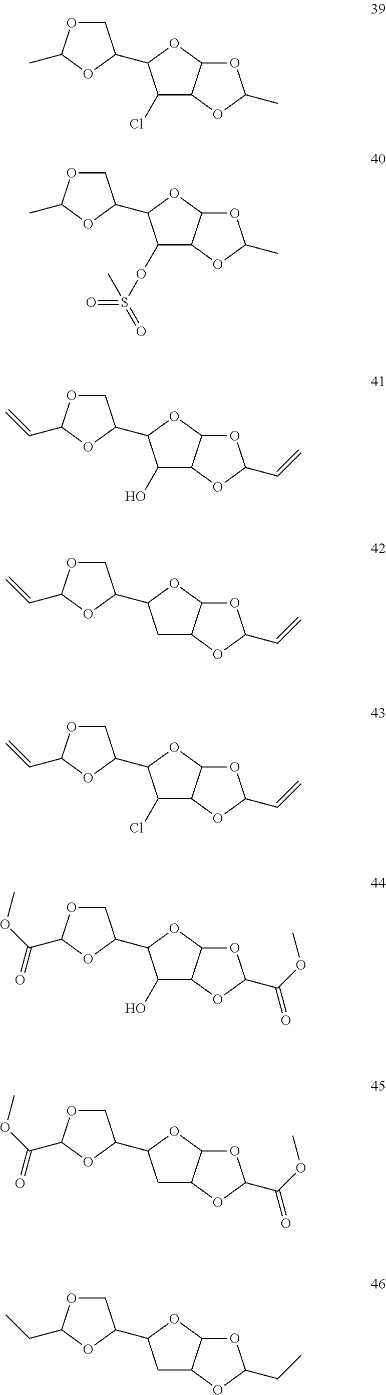

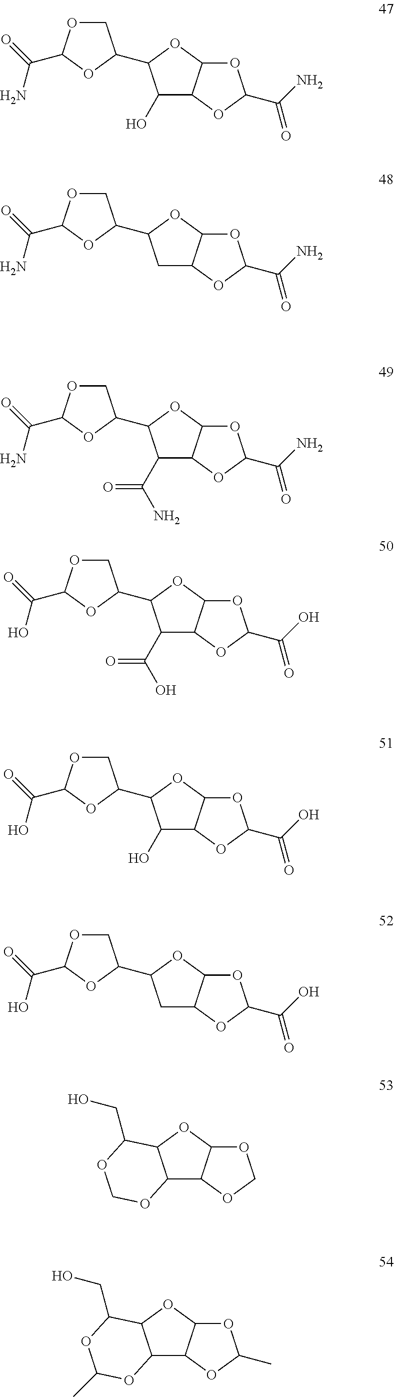

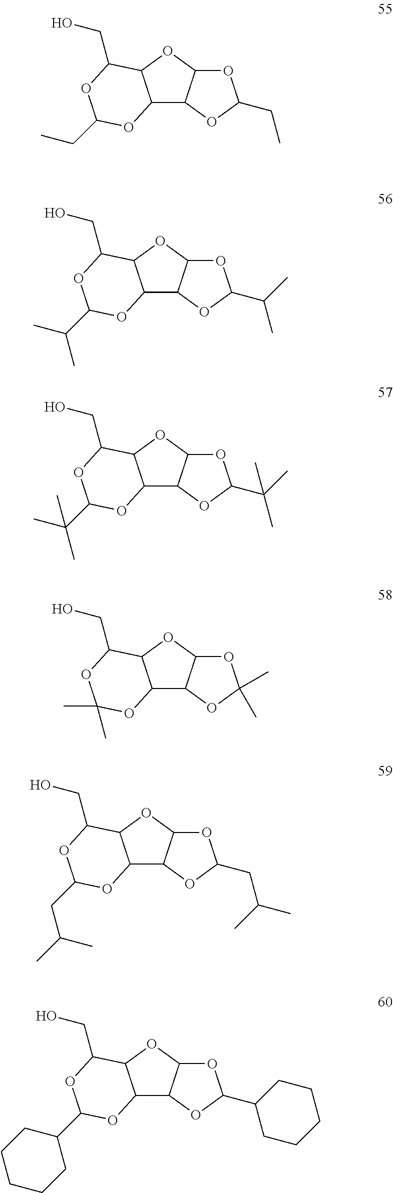

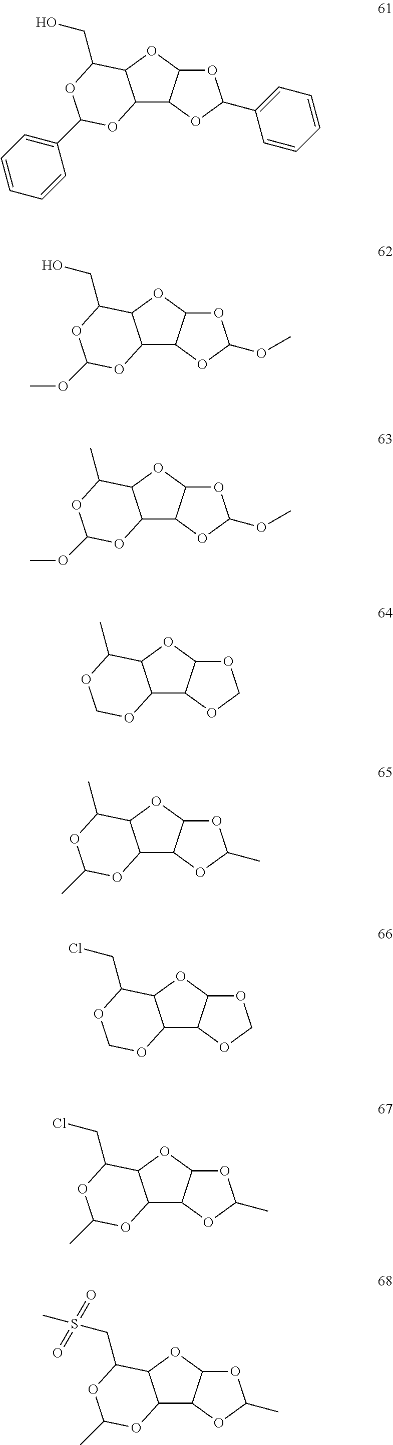

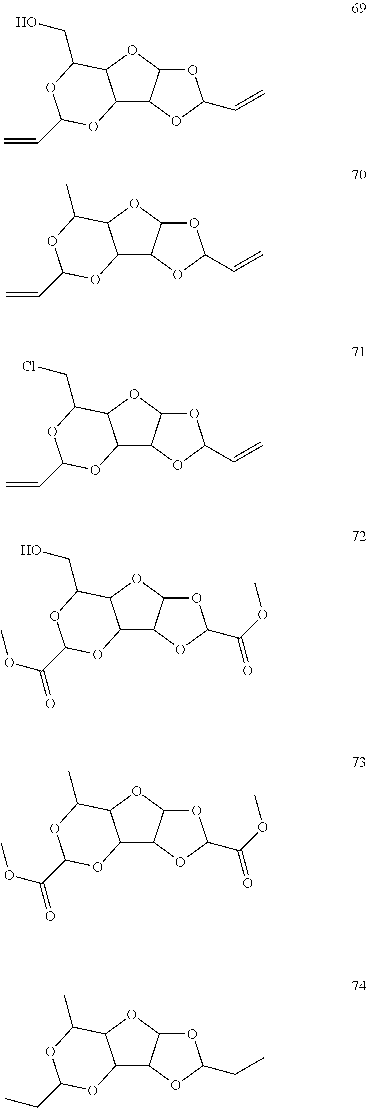

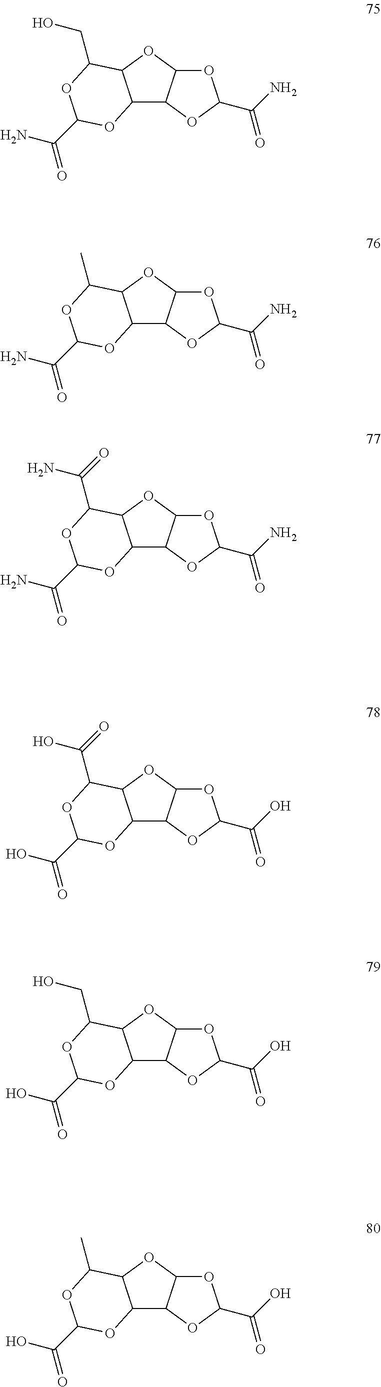 [SMILES: NC(=O)C1OC2OC3C(CO)OC(C(N)=O)OC3C2O1 ; CC1OC(C(N)=O)OC2C1OC1OC(C(N)=O)OC12 ; NC(=O)C1OC2OC3C(C(N)=O)OC(C(N)=O)OC3C2O1 ; O=C(O)C1OC2OC3C(C(=O)O)OC(C(=O)O)OC3C2O1 ; O=C(O)C1OC2OC3C(CO)OC(C(=O)O)OC3C2O1 ; CC1OC(C(=O)O)OC2C1OC1OC(C(=O)O)OC12]

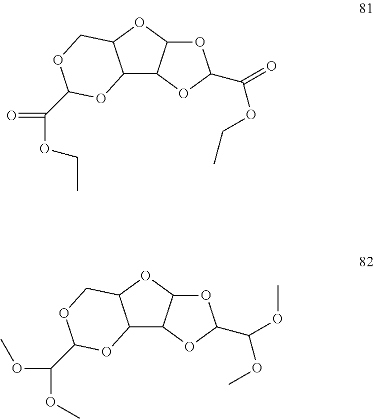

14. Use according to claim 1, wherein the compound of the general formula (I), (II) (III), (IV), (V) and (VI) is used
as solvent in enzymatic reactions, in particular for a polycondensations for the synthesis of polyesters, or
as at least one component of battery electrodes,
for synthesis of metal-organic frameworks (MOFs)
for preparation of electrodes,
as solvent for liquid-phase exfoliations, in particular as substitute for NMP in liquid exfoliation of MoS₂ particles,
solvent for storage,
solvent for biomass pretreatment and biomass processing, in particular for aldehyde-assisted biomass fractionation,
for solid-Phase Peptide Synthesis, in particular as replacement for N,N-dimethylacetamide (DMAc) in solid-Phase Peptide Synthesis,
for Microfiltration membrane preparation,
component of Deep eutectic solvents.

15. Use according to claim 1, wherein the compounds of the general formula (I) or (II) are used as a replacement of N-methylpyrrolidinone, N,N-dimethylacetamide (DMAc) or N,N-dimethylformamide (DMF), 1,3-dimethyl-2-imidazolidinone (DMI) for chemical reaction, in particular for organic synthesis.

16. Use according to claim 1, wherein the compound is Diformylxylose (DFX)

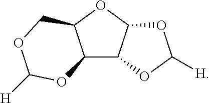

17. Method for producing a compound of the general formula (I), (II), (III) (VI), (V) or (VI) 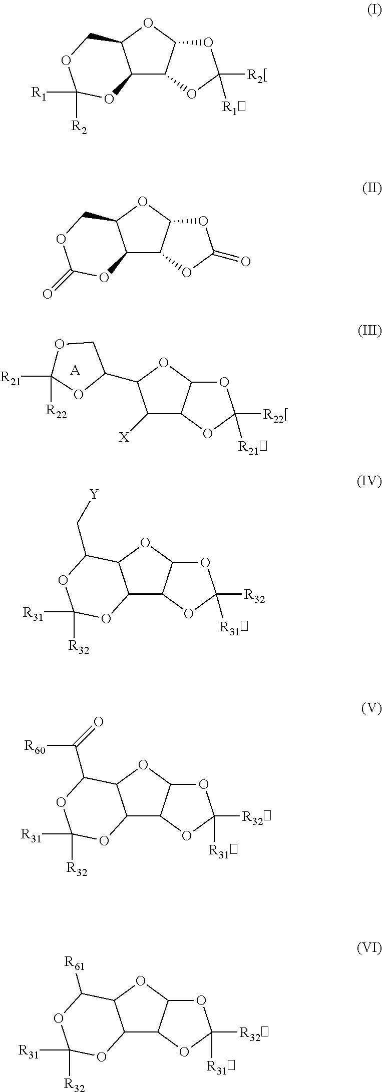
wherein R₁ and R₁′, R₂₁ and R₂₁′, R₃₁ and R₃₁′ are the same and are hydrogen, a linear or branched C₁ to C₁₈ alkyl, a linear or branched C₂ to C₁₈ alkenyl, a linear or branched C₁ to C₁₀ alkoxy, a linear or branched C₁ to C₉ alkanoyloxy, a linear or branched C₁ to C₉ alkoxycarbonyl, aminocarbonyl, hydroxycarbonyl, a linear or branched C₁ to C₉ alkoxyalkyl, an unsubstituted cycloalkyl, a linear or branched C₁ to C₆ alkyl substituted cycloalkyl, preferably an unsubstituted cyclohexyl, an unsubstituted C₆ to C₁₂ aryl, a linear or branched C₁ to C₄ alkyl substituted C₆ to C₁₂ aryl, or C₇ to C₁₂ aralkyl,
R₂ and R₂′, R₂₂ and R₂₂′, R₃₂ and R₃₂′ are the same and are hydrogen, a linear or branched C₁ to C₁₈ alkyl, a linear or branched C₁ to C₉ alkoxyalkyl, or R₂ and R₂′ form together with R₁ and R₁′ respectively, R₂₂ and R₂₂′ form together with R₂₁ and R₂₁′ respectively, R₃₂ and R₃₂′ form together with R₁ and R₁′ respectively, a 5 or 6 membered unsaturated or saturated carbocyclic ring optionally comprising 1 or 2 oxygen atoms,
X is selected from the group consisting of hydrogen, fluoro, chloro, bromo, iodo, alkanoyloxy, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl,
Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl,
R₆₀ is selected from the group consisting of hydrogen, hydroxy and C₁ to C₁₈ alkoxy, and
R₆₁ is selected from the group consisting of hydrogen, hydroxy, C₁ to C₁₀ alkylsulfonyl-C₁ to C₅ and C₁ to C₁₈ alkyl,
involving the reaction step of
reacting D-xylose or D-glucose with
an aldehyde of the general formula (XXX) R₁₁CHO, (XXXI) R₂₁CHO or (XXXII) R₃₁CHO, or
a ketone of the general formula (XXIII) R₁₁COR₁₂, (XXIV) R₂₁COR₂₂ or (XXV) R₃₁COR₃₂ or
a cyclic or linear carbonate
and R₁₁, R₁₂, R₂₁, R₂₂, R₃₁ and R₃₂ have the same definition as above,
in the presence of H₂SO₄ using 2-methyltetrahydrofuran (2-Me-THF) as a solvent.

18. Method according to claim 17, wherein the aldehyde is paraformaldehyde.

19. Method according to claim 17 comprising the steps of
a) providing a composition containing D-xylose or D-glucose, an aldehyde and 2-methyltetrahydrofuran (2-Me-THF),
b) adding H₂SO₄ to the composition of step a)
c) heating the composition of step b), preferably to a temperature between 60 and 120° C.

20. Method according to claim 17, comprising the steps of
d) extracting the compound of the general formula (I), (II), (III) (VI), (V) or (VI) using at least one extraction solvent and
e) distilling the product from the extracted organic residue,
wherein the extraction solvent is ethyl acetate or cyclopentyl methyl ether (CPME).

21. Method according to claim 17, wherein the compound is selected from the group consisting of

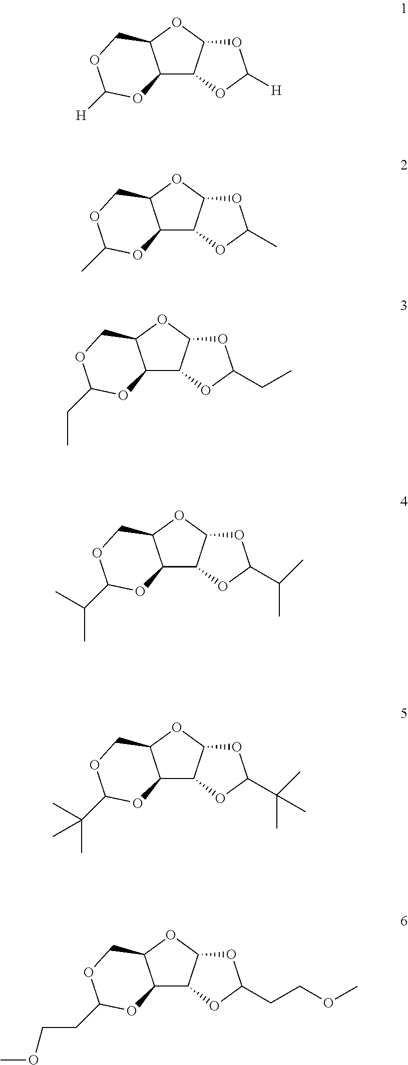

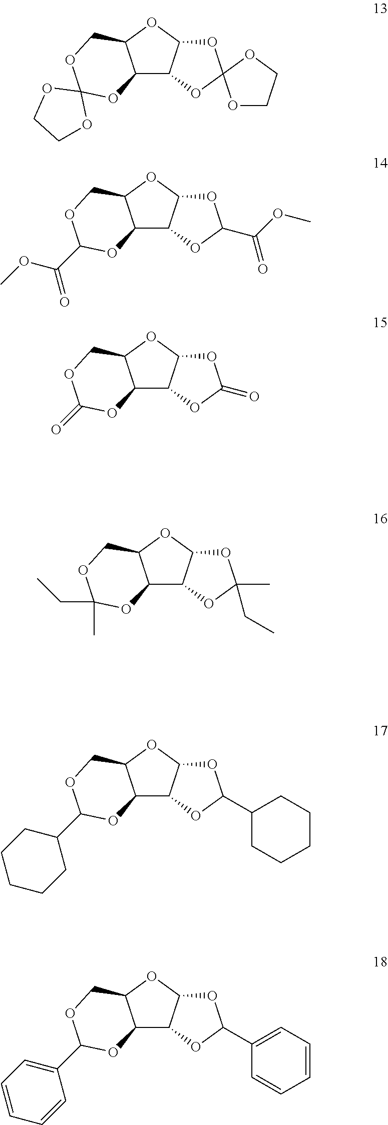

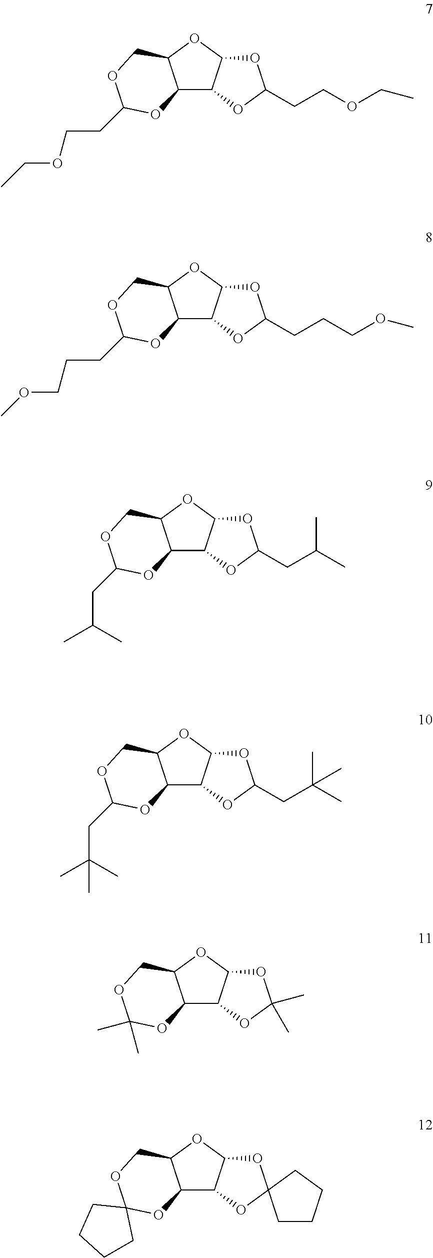

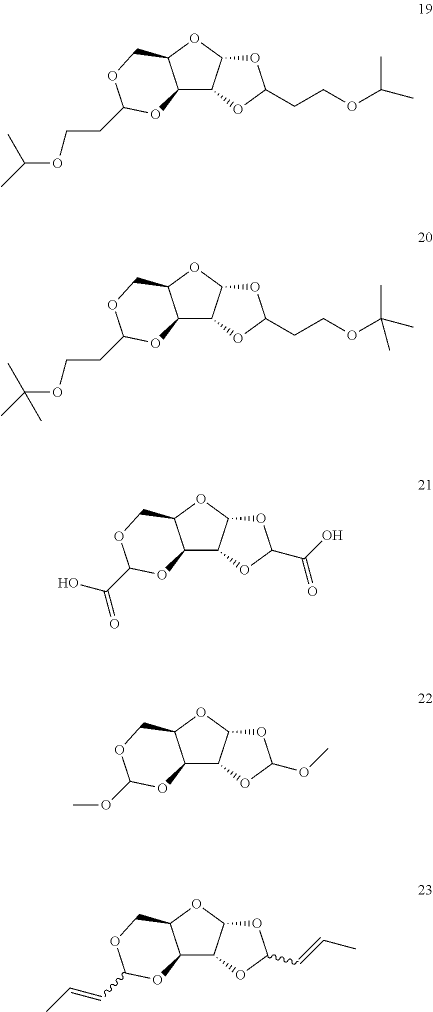

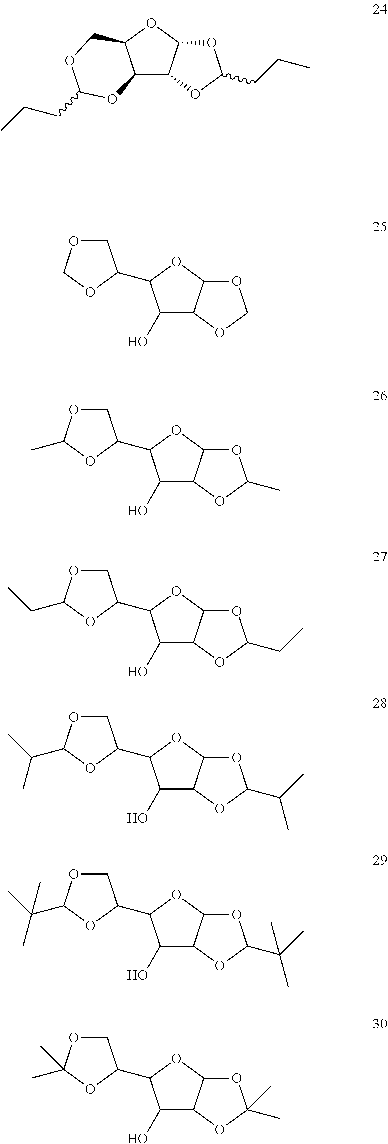

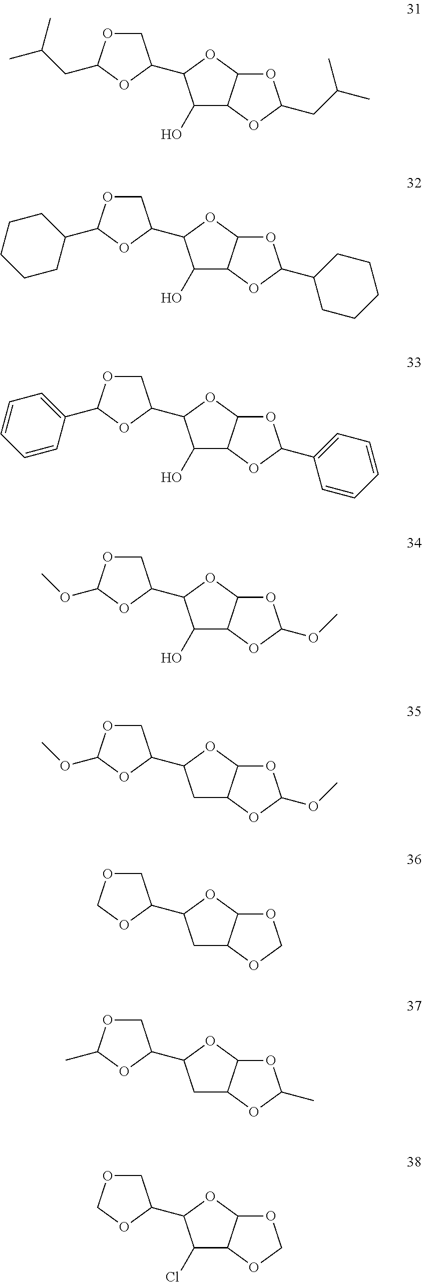

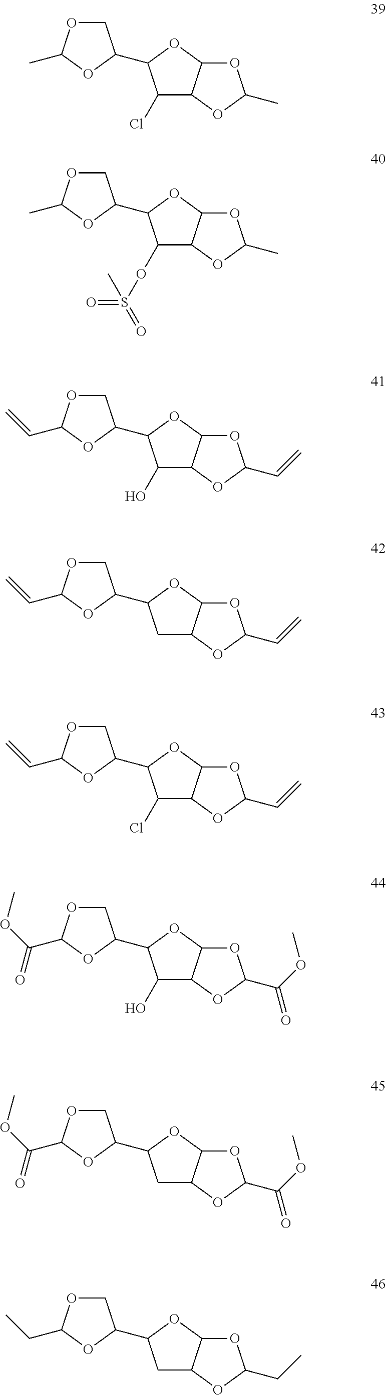

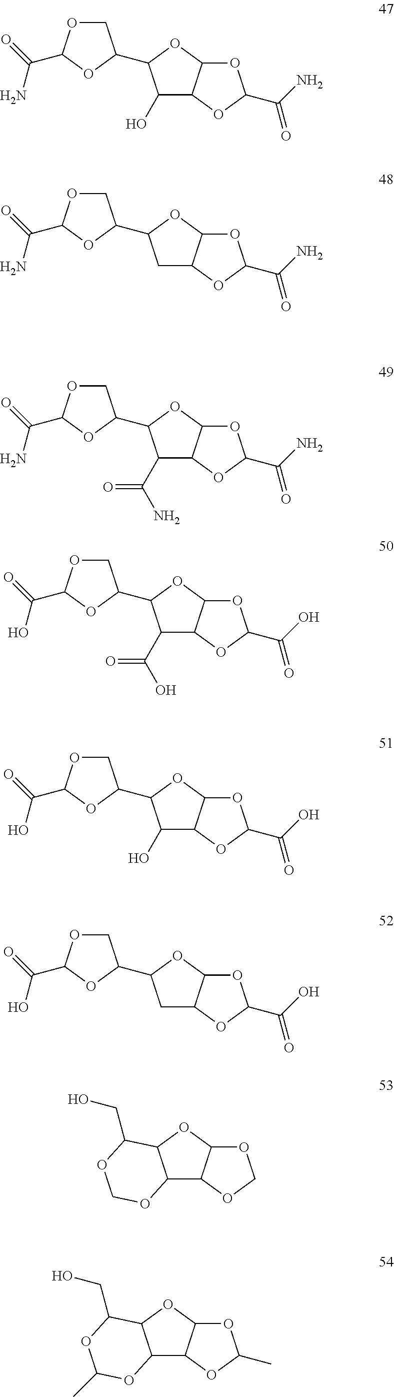

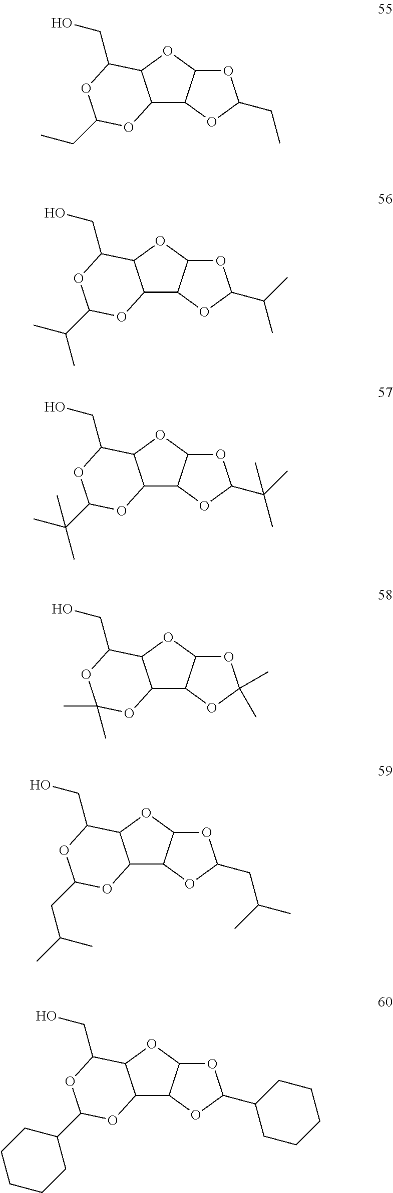

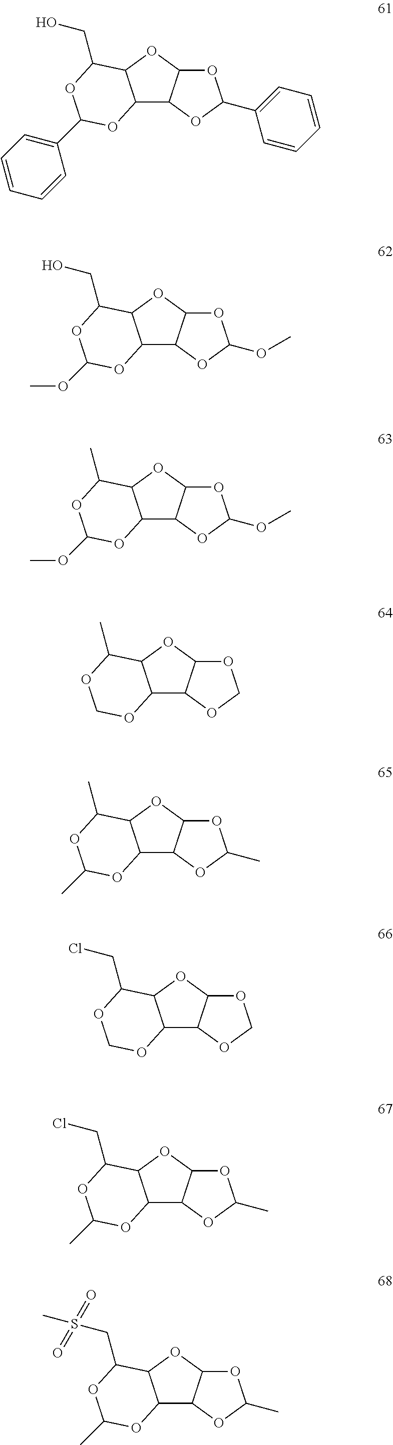

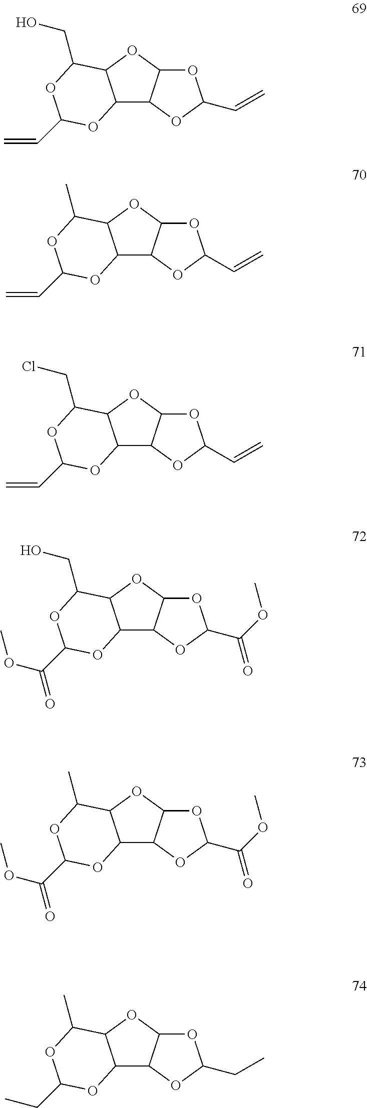

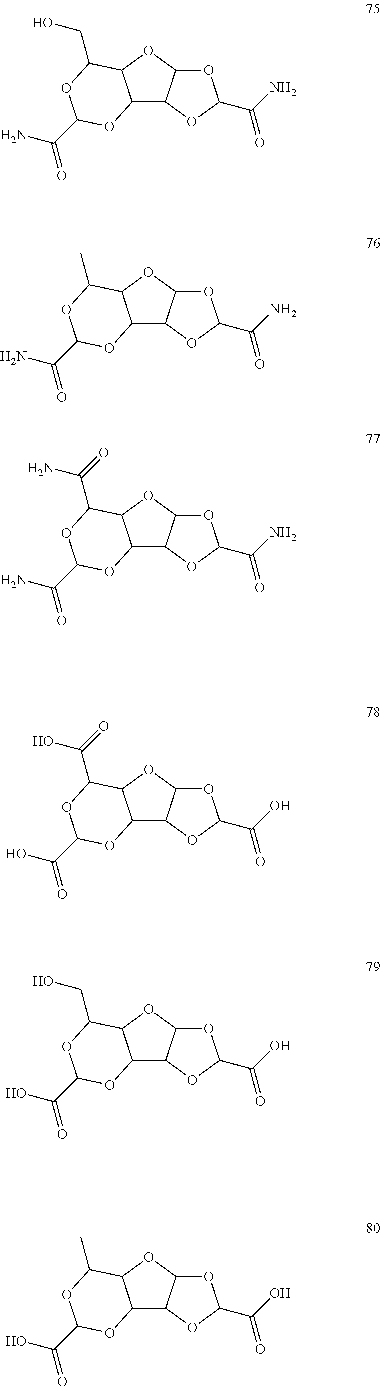 [SMILES: NC(=O)C1OC2OC3C(CO)OC(C(N)=O)OC3C2O1 ; CC1OC(C(N)=O)OC2C1OC1OC(C(N)=O)OC12 ; NC(=O)C1OC2OC3C(C(N)=O)OC(C(N)=O)OC3C2O1 ; O=C(O)C1OC2OC3C(C(=O)O)OC(C(=O)O)OC3C2O1 ; O=C(O)C1OC2OC3C(CO)OC(C(=O)O)OC3C2O1 ; CC1OC(C(=O)O)OC2C1OC1OC(C(=O)O)OC12]

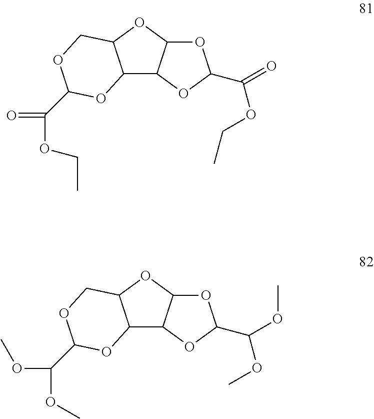

22. Method according to claim 17, wherein the compound is Diformylxylose (DFX)

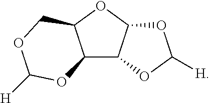

23. Compound of the general formula (I), (III) or (VI) 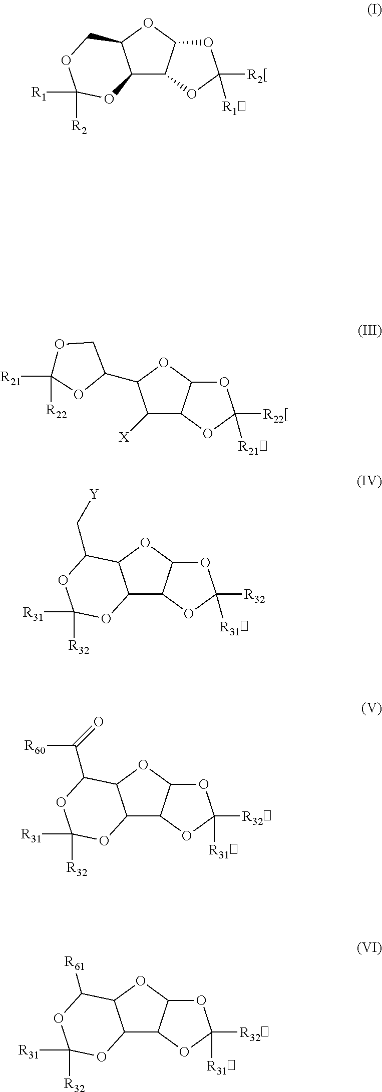
wherein R₁ and R₁′, R₂₁ and R₂₁′, R₃₁ and R₃₁′ are the same and are a linear or branched C₁ to C₁₈ alkyl, a linear or branched C₂ to C₁₈ alkenyl, a linear or branched C₁ to C₁₀ alkoxy, a linear or branched C₁ to C₉ alkoxycarbonyl, aminocarbonyl, hydroxycarbonyl, a linear or branched C₁ to C₉ alkoxyalkyl, an unsubstituted cycloalkyl, a linear or branched C₁ to C₆ alkyl substituted cycloalkyl, preferably an unsubstituted cyclohexyl, an unsubstituted C₆ to C₁₂ aryl, a linear or branched C₁ to C₄ alkyl substituted C₆ to C₁₂ aryl, or C₇ to C₁₂ aralkyl,
R₂ and R₂′, R₂₂ and R₂₂′, R₃₂ and R₃₂′ are the same and are hydrogen, a linear or branched C₁ to C₁₈ alkyl, a linear or branched C₁ to C₉ alkoxyalkyl, or R₂ and R₂′ form together with R₁ and R₁′ respectively, R₂₂ and R₂₂′ form together with R₂₁ and R₂₁′ respectively, R₃₂ and R₃₂′ form together with R₁ and R₁′ respectively, a 5 or 6 membered unsaturated or saturated carbocyclic ring optionally comprising 1 or 2 oxygen atoms,
X is selected from the group consisting of hydrogen, fluoro, chloro, bromo, iodo, alkoxycarbonyl, aminocarbonyl, hydroxycarbonyl, a linear or branched C₁ to C₁₈ alkyl, a C₁ to C₁₈ alkenyl, a linear or branched C₂ to C₁₀ alkenyl, a sulfonate and OR₅₀, wherein R₅₀ is selected from the group consisting of hydrogen, a linear or branched C₁ to C₁₈ alkyl, and a linear or branched C₁ to C₉ alkylcarbonyl, and
Y is selected from the group consisting of hydrogen, fluoro, chloro, bromo, iodo, a linear or branched C₁ to C₁₈ alkyl, a C₁ to C₁₈ alkenyl, a linear or branched C₂ to C₁₀ alkenyl, a sulfonate and OR₅₀, wherein R₅₀ is hydrogen, a linear or branched C₁ to C₁₈ alkyl and a linear or branched C₁ to C₉ alkylcarbonyl,
R₆₀ is selected from the group consisting of hydrogen, hydroxy and C₁ to C₁₈ alkoxy, and
R₆₁ is selected from the group consisting of hydrogen, hydroxy, C₁ to C₁₀ alkylsulfonyl-C₁ to C₅, alkylsulfonylalkenyl and C₁ to C₁₈ alkyl
with the proviso that compounds (1), (10), (11), (12) and (14) 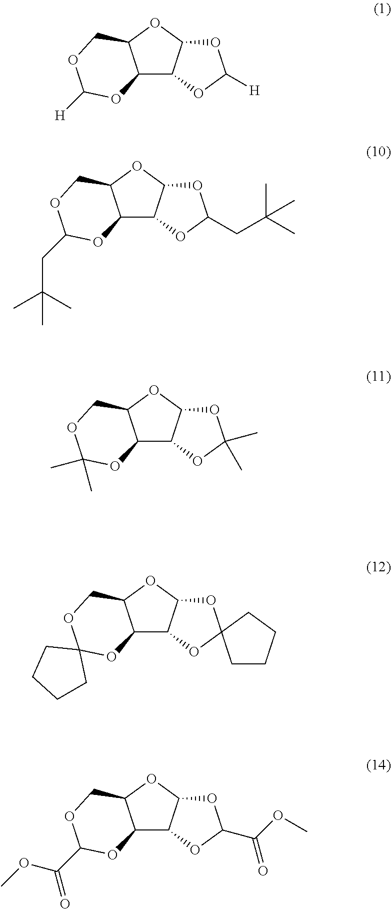
are excluded.
